(19) United States
(12) Patent Application Publication
(10) Pub. No.: US 2021/0137941 A1
(43) Pub. Date: May 13, 2021

(54) CLOZAPINE FOR THE TREATMENT OF IG-E DRIVEN B CELL DISEASES

(71) Applicant: ZARODEX THERAPEUTICS LIMITED, London (GB)

(72) Inventors: Stephen JOLLES, Cardiff (GB); Houman ASHRAFIAN, London (GB); Duncan MCHALE, London (GB)

(73) Assignee: ZARODEX THERAPEUTICS LIMITED, London (GB)

(21) Appl. No.: 16/965,269

(22) PCT Filed: Jan. 31, 2019
(86) PCT No.: PCT/EP2019/052451

§ 371 Date: Jul. 27, 2020

(30) Foreign Application Priority Data
Jan. 31, 2018 (EP) 18154550.0

(51) Int. Cl. A61K 31/5513; A61P 25/18

(52) U.S. Cl. CPC A61K 31/5513; A61P 25/18

(57) ABSTRACT

This invention relates to the compound clozapine and its major metabolite norclozapine and prodrugs thereof and pharmaceutically acceptable salts and solvates thereof for use in the treatment or prevention of a pathogenic IgE driven B cell disease. The invention also provides pharmaceutical compositions containing such compounds.

### TECHNICAL FIELD

This invention relates to a compound and pharmaceutical compositions containing such compound for use in the treatment or prevention of pathogenic IgE driven B cell diseases.

### BACKGROUND TO THE INVENTION

The compound associated with this invention is known as clozapine i.e. the compound of the following structure:

[chemical structure]

Clozapine has a major active metabolite known as norclozapine (Guitton et al., 1999) which has the following structure:

[chemical structure]

Clozapine is known as a treatment for resistant schizophrenia. Schizophrenia is an enduring major psychiatric disorder affecting around 1% of the population. Apart from the debilitating psychiatric symptoms it has serious psychosocial consequences with an unemployment rate of 80-90% and a life expectancy reduced by 10-20 years. The rate of suicide among people with schizophrenia is much higher than in the general population and approximately 5% of those diagnosed with schizophrenia commit suicide.

Clozapine is an important therapeutic agent and is included on the WHO list of essential medicines. It is a dibenzo-diazepine atypical antipsychotic, and since 1990 the only licensed therapy in the UK for the 30% of patients with treatment-resistant schizophrenia (TRS). It shows superior efficacy in reducing both positive and negative symptoms in schizophrenic patients and is effective in approximately 60% of previously treatment refractive patients with a significant reduction in suicide risk. The National Institute for Health and Clinical Excellence (NICE) guideline recommends adults with schizophrenia which has not responded adequately to treatment with at least 2 antipsychotic drugs (at least one of which should be a non-clozapine second generation antipsychotic) should be offered clozapine.

Clozapine is associated with serious adverse effects including seizures, intestinal obstruction, diabetes, thromboembolism, cardiomyopathy and sudden cardiac death. It can also cause agranulocytosis (cumulative incidence 0.8%); necessitating intensive centralised registry based monitoring systems to support its safe use. In the UK there are three electronic registries (www.clozaril.co.uk, www.denzapine.co.uk and www.ztas.co.uk) one for each of the clozapine suppliers. Mandatory blood testing is required weekly for the first 18 weeks, then every two weeks from weeks 19-52 and thereafter monthly with a ‘red flag’ cut-off value for absolute neutrophil count (ANC) of less than 1500/4 for treatment interruption.

In 2015, the Federal Drug Administration (FDA) merged and replaced the six existing clozapine registries in the United States combining data from over 50,000 prescribeics, 28,000 pharmacies and 90,000 patients records into a single shared registry for all clozapine products, the Clozapine Risk Evaluation and Mitigation Strategy (REMS) Program (www.clozapinerems.com). Changes were introduced lowering the absolute neutrophil count (ANC) threshold to interrupt clozapine treatment at less than 1000/μL in general, and at less than 500/μL in benign ethnic neutropenia (BEN). Prescribers have greater flexibility to make patient-specific decisions about continuing or resuming treatment in patients who develop moderate to severe neutropenia, and so maximize patient benefit from access to clozapine.

Schizophrenia is associated with a 3.5 fold increased chance of early death compared to the general population. This is often due to physical illness, in particular chronic obstructive pulmonary disease (COPD) (Standardised Mortality Ratio (SMR) 9.9), influenza and pneumonia (SMR 7.0). Although clozapine reduces overall mortality in severe schizophrenia, there is a growing body of evidence linking clozapine with elevated rates of pneumonia-related admission and mortality. In an analysis of 33,024 patients with schizophrenia, the association between second generation antipsychotic medications and risk of pneumonia requiring hospitalization was highest for clozapine with an adjusted risk ratio of 3.18 with a further significant increase in risk associated with dual antipsychotic use (Kuo et al., 2013). Although quetiapine, olanzapine, zotepine, and risperidone were associated with a modestly increased risk, there was no clear dose-dependent relationship and the risk was not significant at time points beyond 30 days (Leung et al., 2017; Stoecker et al., 2017).

In a 12 year study of patients taking clozapine, 104 patients had 248 hospital admissions during the study period. The predominant admission types were for treatment of either pulmonary (32.2%) or gastrointestinal (19.8%) illnesses. The commonest pulmonary diagnosis was pneumonia, (58% of pulmonary admissions) and these admissions were unrelated to boxed warnings (Leung et al., 2017).

In a further nested case control study clozapine was found to be the only antipsychotic with a clear dose-dependent risk for recurrent pneumonia, this risk increased on re-exposure to clozapine (Hung et al., 2016).

While these studies underscore the increased admissions or deaths from pneumonia and sepsis in patients taking clozapine over other antipsychotics, the focus on extreme outcomes (death and pneumonia) may underestimate the burden of less severe but more frequent infections such as sinusitis, skin, eye, ear or throat infections and community acquired and treated pneumonia. Infection may represent an important additional factor in destabilizing schizophrenia control and clozapine levels.

Various mechanisms for the increase in pneumonia have been suggested, including aspiration, sialorrhoea and impairment of swallowing function with oesophageal dilatation, hypomotility and agranulocytosis. In addition, cigarette smoking is highly prevalent among patients with schizophrenia as a whole and represents an independent risk factor for pneumonia incidence and severity (Bello et al., 2014) (Bello et al., 2014) (Bello et al., 2014) (Bello et al., 2014) (Bello et al., 2014) (Bello et al., 2014) (Bello et al., 2014) (Bello et al., 2014).

A small amount of research into the immunomodulatory properties of clozapine has been performed:

Hinze-Selch et al (Hinze-Selch et al., 1998) describes clozapine as an atypical antipsychotic agent with immunomodulatory properties. This paper reports that patients that received clozapine treatment for six weeks showed significant increases in the serum concentrations of IgG, but no significant effect was found on IgA or IgM concentrations or on the pattern of autoantibodies.

Jolles et al (Jolles et al., 2014) reports studies on the parameter “calculated globulin (CG)” as a screening test for antibody deficiency. Patients with a wide range of backgrounds were selected from thirteen laboratories across Wales. Of the patients with significant antibody deficiency (IgG <4 g/L, reference range 6-16 g/L), identified on CG screening from primary care, clozapine use was mentioned on the request form in 13% of the samples. However, antibody deficiency is not a listed side effect of clozapine in the British National Formulary (BNF), nor does antibody testing constitute part of current clozapine monitoring protocols.

Another study by Lozano et al. (Lozano et al., 2016) reported an overall decrease of mean plasma levels of IgM in the study group (which consisted of psychiatric outpatients who took clozapine for at least five years) compared to the control group, and also reported that no differences were found between the groups with respect to IgA, IgG, absolute neutrophil count and white blood cell count.

Consequently, given these mixed results that have been reported, the immunomodulatory properties of clozapine and its effect on immunoglobin levels, particularly IgE, are neither clear nor understood in the art.

Pathogenic IgE immunoglobulin driven diseases result from secretion of autoantibodies (including IgE) by antibody secreting cells (ASCs, collectively plasmablasts and plasma cells these being types of mature B cell). These antibodies target a variety of exogenous antigens causing an exaggerated response which have been characterised in many of these conditions. There is rarely an increase in overall immunoglobulins as the pathological process is driven by the secretion of specific immunoglobulins which constitute a small percentage of the total immunoglobulins. Secretion of IgE antibodies is from ASCs, and ASCs are generated secondary to the differentiation of class-switched and unswitched memory B cells these being further types of mature B cell. Various lines of evidence suggest this is a highly-dynamic process, with ongoing differentiation occurring almost constantly.

Class-switched memory B cells are mature B cells that have replaced their primary encoded membrane receptor [IgM] by IgG, IgA or IgE in response to repeated antigen recognition. This class-switching process is a key feature of normal humoral immunological memory, both ‘constitutive’ through the secretion of pre-existing protective antibodies by long-lived plasma cells, and ‘reactive’ reflecting re-exposure to antigen and reactivation of memory B cells to either differentiate into plasma cells to produce antibodies, or to germinal centre B cells to enable further diversification and affinity maturation of the antibody response. Early in the immune response, plasma cells derive from unswitched activated B cells and secrete IgM. Later in the immune response, plasma cells originate from activated B cells participating in the germinal centre (areas forming in secondary lymphoid follicular tissue in response to antigenic challenge) which have undergone class switching (retaining antigen specificity but exchanging immunoglobulin isotype) and B cell receptor (BCR) diversification through immunoglobulin somatic hypermutation. This maturation process enables the generation of BCRs with high affinity to antigen and production of different immunoglobulin isotypes (i.e. exchanging the originally expressed IgM and IgD to IgG, IgA or IgE isotypes) (Budeus et al., 2015; Kracker and Durandy, 2011).

Class switch recombination (CSR) following the germinal centre reaction in secondary lymphoid organs provides antigen-primed/experienced autoreactive memory B cells and a core pathway for development and/or maintenance of autoimmunity. Post-germinal centre B cells class-switched to IgG or IgA in the periphery can enter other anatomic compartments, such as the central nervous system, to undergo further affinity maturation (e.g. in tertiary lymphoid structures in multiple sclerosis) and contribute to immune pathology (Palanichamy et al., 2014). CSR can occur locally within tissue in pathology, such as within ectopic lymphoid structures in chronically inflamed tissue such as rheumatoid arthritis synovium (Alsaleh et al., 2011; Humby et al., 2009).

Notably, plasma cells (in addition to plasmablasts) are increasingly understood to exert important effector immune functions beyond the production of immunoglobulin, including generation of cytokines (Shen and Fillatreau, 2015) and immunoregulators such as tumour-necrosis factor-α (TNF-α), inducible nitric oxide synthase (iNOS) (Fritz et al., 2011), IL-10 (Matsumoto et al., 2014; Rojas et al., 2019), IL-35 (Shen et al., 2014), IL-17a (Bermejo et al., 2013) and ISG15 (Care et al., 2016).

Plasmablasts, representing short-lived rapidly cycling antibody-secreting cells of the B cell lineage with migratory capacity, are also precursors to long-lived (post-mitotic) plasma cells, including those which home in to the bone marrow niche (Nutt et al., 2015). In addition to being precursors of autoreactive long-lived plasma cells, plasmablasts are an important potential therapeutic target themselves through their ability to produce pathogenic immunoglobulin/autoantibody (Hoyer et al., 2004). In addition to their direct antibody secreting function, circulating plasmablasts also exert activity to potentiate germinal centre-derived immune responses and thereby antibody production via a feed-forward mechanism involving Il-6-induced promotion of T follicular helper cell (Tfh) differentiation and expansion (Chavele et al., 2015).

Long-lived plasma cells, whose primary residency niche is in bone marrow (Benner et al., 1981), are thought to be the major source of stable autoantibody production in (both physiologic) and pathogenic states and are resistant to glucocorticoids, conventional immunosuppressive and B cell depleting therapies (Hiepe et al., 2011). Substantiating the critical importance of this B cell population to long-term antibody production, site-specific survival of bone marrow-derived plasma cells with durable (up to 10 years post-immunisation) antibody responses to prior antigens has been demonstrated in non-human primates despite sustained memory B cell depletion (Hammarlund et al., 2017). Given the key role played by autoreactive long-lived plasma cells in the maintenance of autoimmunity (Mumtaz et al., 2012)—and the substantial refractoriness of the autoreactive memory formed by these cells to conventional immunosuppressive agents such as anti-TNF or B cell depleting biologics (Hiepe et al., 2011)

CD19(+) B cells and CD19(−) B plasma cells are drivers of pathogenic IgE driven B cell diseases. Pathogenic IgE driven B cell diseases represent a substantial proportion of all autoimmune and inflammatory diseases. The most prominent, but not the sole mechanism through which pathogenic immunoglobulin driven B cells cause disease, is through auto-antibody production.

Pathogenic IgE driven B cell diseases are poorly treated and as a result they have substantial mortality and morbidity rates, even for the “benign” diseases. Certain current advanced therapies are directed at mature B cells. For example, belimumab is a human monoclonal antibody that inhibits B cell activating factor. Atacicept is a recombinant fusion protein that also inhibits B cell activating factor. However, memory B cells may be resistant to therapies such as belimumab or atacicept which target survival signals such as B cell activation factor (Stohl et al., 2012). The importance of memory B cells in the pathogenesis of autoimmune disorders was also demonstrated by the lack of efficacy of atacicept in treating rheumatoid arthritis and multiple sclerosis (Kappos et al., 2014; Richez et al., 2014). Plasmapheresis and immunoabsorption involve the removal of disease-causing autoantibodies from the patient's bloodstream. However, these treatments have limited efficacy or are complex and costly to deliver. CAR-T methods directed at CD19(+) B cells leaves CD19(−) B plasma cells intact, which makes it ineffective. The effect of rituximab on IgE levels is modest and no sustained clinical benefit has been observed (van Vollenhoven et al., 2013).

Omalizumab (anti-IgE antibody) is presently indicated for the treatment of asthma. It is, however, an expensive medicine.

Thus, there is a major unmet medical need for new treatments against pathogenic IgE driven B cell diseases.

### SUMMARY OF THE INVENTION

### Impact on Class-Switched Memory B Cells and Antibody Production

It has been found by the inventors that clozapine has a potential important therapeutic effect as it significantly reduces class switched memory B cells (“CSMB”), a type of mature B cell.

Reduction in CSMBs by clozapine will consequently reduce the numbers of ASCs, and hence the secretion of specific immunoglobulins including the pathogenic immunoglobulins. Clozapine was also observed to cause a reduction in levels of plasmablasts, another type of mature B cell. This functional effect on persistent and long lived adaptive B cell and plasma cell function may ameliorate the diseases driven by the persistent generation of pathogenic immunoglobulins that drives the pathology of pathogenic IgE driven B cell diseases. This will consequently reduce the numbers of ASCs, and hence the secretion of specific immunoglobulins including the pathogenic immunoglobulins. Clozapine was also observed to cause a reduction in levels of plasmablasts, another type of mature B cell. This functional effect on persistent and long lived adaptive B cell and plasma cell function may ameliorate the diseases driven by the persistent generation of pathogenic immunoglobulins that drives the pathology of pathogenic IgE driven B cell diseases. Further, the inventors' new data demonstrates both a very significant effect on the number of circulating class switched memory B cells, a substantial effect on the number of plasmablasts and importantly, through the lack of recall response to common vaccines, an effect on the function of the class switched memory B cells and plasmablasts resulting in specific reduction of antibodies targeting a previously exposed antigen. The inventors' new data also demonstrates an effect of the drug in reducing total immunoglobulin levels after administration. With the lack of effect on other B cells, shown by the lack of depletion of other sub-types and total B cell numbers but with a particular reduction in CSMBs and plasmablasts, this observation strongly supports a functional effect on CSMBs and plasmablasts which are central to long lived production of pathogenic antibodies in pathogenic IgE driven B cell diseases.

The inventors' finding of a marked reduction in class-switched memory B cells in patients treated with clozapine indicates a robust impact on the process of immunoglobulin class switching. This has particular therapeutic relevance in pathogenic immunoglobulin driven B cell diseases in which class switch recombination (CSR) following the germinal centre reaction in secondary lymphoid organs provides antigen-primed/experienced autoreactive memory B cells and a core pathway for development and/or maintenance of autoimmunity. The effect of clozapine to both impact on CSR and lower immunoglobulin is of especial therapeutic potential in the setting of pathogenic immunoglobulin-driven B cell diseases where an impact on both the autoimmune memory repertoire and pathogenic immunoglobulin is desirable.

The inventors' identification of a significant impact of clozapine on plasma cell populations indicates the clear potential to modulate the diverse antibody-independent effector functions of B cells relevant to (auto)immune-mediated disease also.

### Impact on Plasmablast Antibody-Secreting Cells

The inventors have found that clozapine exerts a profound effect on reducing levels of circulating plasmablasts in patients. Accordingly, the inventors' observation of a profound impact of clozapine use on circulating plasmablast number highlights the potential for clozapine to modulate pathogenic immunoglobulin-driven B cell disease through both effects on circulating plasmablast secretion of immunoglobulin as well as interference with the potent function of plasmablasts to promote Tfh (T follicular helper) cell function.

### Impact on Long-Lived Plasma Cells

Using a wild type murine model, the inventors have found that regular clozapine administration in mice significantly reduces the proportion of long-lived plasma cells in bone marrow, an effect not seen with use of a comparator antipsychotic agent (haloperidol). Notably, human bone marrow resident long-lived PCs are long-regarded as the primary source of circulating Ig in human, thus providing a clear substrate for the inventors' observation of reduction in Ig in patients treated with clozapine. The inventors' observation of a specific effect of clozapine to deplete bone marrow long-lived plasma cells has, via an impact on long-lived plasma cell (autoreactive) memory, substantial therapeutic potential in pathogenic immunoglobulin driven B cell disease to eliminate inflammation and achieve remission.

The inventors' identification of a significant impact of clozapine on plasma cell populations indicates the clear potential to modulate the diverse antibody-independent effector functions of B cells relevant to (auto)immune-mediated disease also.

### Impact on B Cell Precursors in Bone Marrow and Splenic Immature/Transitional Cells

The inventors identify a clear impact of clozapine on bone marrow B cell precursors after dosing of wild type mice. Specifically, an increase in the proportion of pre-pro B cells, in conjunction with a reduction in pre-B cells, proliferating pre-B cells and immature B cells in bone marrow. Together, these findings suggest a specific impact of clozapine on early B cell development, with a partial arrest between the pre-pro-B cell and pre-B cell stages in the absence of specific immunological challenge. The inventors have discerned an impact of clozapine to reduce the proportion of splenic T1 cells in wild type mice. Mirroring the murine findings, the inventors' interim findings from an ongoing observational study of patients on clozapine reveal a significant reduction in circulating transitional B cells. The human circulating transitional B cell subpopulation exhibits a phenotype most similar to murine T1 B cells and is expanded in autoimmune disease.

Accordingly, the inventors' observation of an impact of clozapine to reduce the proportions of bone marrow B cell progenitors and immature (T1) splenic B cells provides additional anatomic compartmental origins beyond germinal centres for their finding of a reduction in circulating class-switched memory B cells and immunoglobulin in patients treated with clozapine. The therapeutic potential of this is further underlined by the consideration that the majority of antibodies expressed by early immature B cells are self-reactive (Wardemann et al., 2003).

### Lack of Direct B Cell Toxicity In Vitro

The inventors' new data using an in vitro B cell differentiation system to assess the specific impact of clozapine, its metabolite (N-desmethylclozapine) and a comparator antipsychotic control drug (haloperidol) further demonstrate: no direct toxicity effect of clozapine or its metabolite on differentiating B cells, no consistent effect on the ability of differentiated ASCs to secrete antibody and no consistent inhibitory effect on functional or phenotypic maturation of activated B cells to an early PC state in the context of an established in vitro assay.

Limited to the context of these in vitro experiments, these data suggest that clozapine is unlikely to be acting in a direct toxic manner on plasma cells or their precursors (e.g. via a cell intrinsic effect) to induce the effects observed on immunoglobulin levels. The observations suggest that clozapine's effect on B cells is more nuanced than existing B cell targeting therapies used for autoimmune disease which result in substantial depletion of multiple B cell subpopulations (e.g. rituximab and other anti-CD20 biosimilars) whose efficacy is mediated via direct effects on B cells such as signalling induced apoptosis, complement-mediated cytotoxicity or antibody-dependent cellular cytotoxicity.

Such a lack of apparent substantial direct toxicity by clozapine has a number of potential therapeutic advantages for clozapine, including reduced risk of generalised immunosuppression associated with indiscriminate B cell depletion (including elimination of protective B cells), and the potential to avoid maladaptive alterations observed with use of conventional B cell depleting therapies.

### Efficacy in Collagen-Induced Arthritis (CIA) Mouse Model and Relevance of CIA as a Model of B Cell Driven Disease Involving Pathogenic Immunoglobulin

CIA is a well-established experimental model of autoimmune disease that results from immunisation of genetically susceptible strains of rodents and non-human primates with type II collagen (CII) (Brand et al., 2004)—a major protein component of cartilage—emulsified in complete Freund's adjuvant. This results in an autoimmune response accompanied by a severe polyarticular arthritis, typically 18-28 days post-immunisation and monophasic, resolving after ^{˜}60 days in mice (Bessis et al., 2017; Brand et al., 2007). The pathology of the CIA model resembles that of rheumatoid arthritis, including synovitis, synovial hyperplasia/pannus formation, cartilage degradation, bony erosions and joint ankylosis (Williams, 2012).

The immunopathogenesis of CIA is dependent on B cell-specific responses with generation of pathogenic autoantibodies to CII, in addition to involving T cell-specific responses to CII, FcγR (i.e. Fc receptors for IgG) and complement. The critical role of B cells in the development of CIA is substantiated by the complete prevention of development of CIA in mice deficient for B cells (IgM deleted), notwithstanding an intact anti-CII T cell response (Svensson et al., 1998). Moreover, the development of CIA has been shown to be absolutely dependent on germinal centre formation by B cells, with anti-CII immunoglobulin responses themselves largely dependent on normal germinal centre formation (Dandah et al., 2018; Endo et al., 2015). B cells have also been implicated in other aspects of CIA pathology, including bone erosion through inhibition of osteoblasts (Sun et al., 2018). As a corollary, B cell depletion using anti-CD20 monoclonal antibodies prior to CII immunisation delays onset and severity of CIA, in conjunction with delayed autoantibody production (Yanaba et al., 2007). In this model, B cell recovery was sufficient to result in pathogenic immunoglobulin production after collagen-immunisation and associated development of disease.

The fundamental role played by collagen-specific autoantibodies in the pathogenesis of CIA are highlighted by the observations that passive transfer of anti-CII serum or polyclonal IgG immunoglobulin to unimmunised animals results in arthritis (Stuart and Dixon, 1983), whilst lack of the FcγR chain near completely abrogates development of CIA in mice (Kleinau et al., 2000). In addition, introduction of pathogenic antibodies (i.e. collagen antibody-induced arthritis, CAIA) into germinal centre-deficient mice results in arthritis, demonstrating the ability of pathogenic antibody to largely circumvent the requirement for the germinal centre reaction (Dandah et al., 2018). Moreover, even mice lacking adaptive immunity (i.e. B and T cells), are susceptible to induction of CIA (Nandakumar et al., 2004).

Accordingly, the inventors have employed the CIA model as a highly clinically relevant experimental system in which B cell-derived pathogenic immunoglobulin made in response to a sample antigen drives autoimmune pathology to explore the potential efficacy of clozapine and its associated cellular mechanisms. The inventors demonstrate that clozapine delays the onset and reduces the incidence of CIA in mice, an effect most apparent when dosed just after CII immunisation. Furthermore, the inventors' data indicates that clozapine reduces the severity of CIA, judged by number of affected paws and clinical severity score. The inventors identify important effects of clozapine on key cell types implicated in the pathogenesis of CIA, including a reduction in the proportion of splenic plasma cells and highly significant reduction in germinal centre B cells in local draining lymph node. Moreover, the inventors' findings demonstrate reduced markers of functional activity for antibody production and antigen presentation on lymph node germinal centre B cells in response to clozapine in CII immunised mice. Measured at a single time point, they also observe a significant reduction in anti-collagen antibody levels. Together, the inventors' findings in the CIA model point to a specific ability of clozapine to favourably impact upon pathogenic immunoglobulin B cell-driven pathology and thereby B cell mediated disorders in which autoantibody formation is a key component.

Importantly, IgE memory B cells and IgE plasma cells have also been shown to develop via a germinal centre pathway (Talay et al., 2012). Notably IgE switch memory B cells are the main source of cellular IgE memory (Talay et al., 2012). Moreover the ontogeny of IgE⁺ B cells and plasma cells follows similar phenotypic stages to that for IgG(1), including IgE⁺ germinal centre-like B cells, IgE⁺ plasmablasts and IgE plasma cells occurring via a sequential switching process from IgG (Ramadani et al., 2017). Notably the intrinsic maturation state of B cells determines their capacity to undergo class switching to IgE, accordingly the highest proportion of IgE cells derive from germinal centre B cells (Ramadani et al., 2017). Furthermore, isotype switching depends on the number of cell divisions and is greater for IgE than IgG (Tangye et al., 2002), consistent with the fact that IgE responses generally require more prolonged antigenic stimulation (Hasbold et al., 1998). Accordingly, the inventors' findings of a specific impact of clozapine on class switching, germinal centre formation and long-lived plasma cells are expected to impact substantially on the ability to mount and sustain an IgE-mediated immunoglobulin response in pathogenic IgE driven B cell diseases. Indeed, the greater number of B cell divisions and requirement for germinal centre B cells to efficiently generate IgE⁺ suggests that these disorders may be particularly susceptible to the effects of clozapine.

Thus, the present invention provides a compound selected from clozapine, norclozapine and prodrugs thereof and pharmaceutically acceptable salts and solvates thereof for use in the treatment or prevention of a pathogenic IgE driven B cell disease in a subject, in particular, wherein said compound causes mature B cells to be inhibited in said subject.

### BRIEF DESCRIPTION OF THE DRAWINGS

FIGS. 1A-C. show the relative frequencies of numbers of patients at each serum concentration value for IgG, IgA and IgM respectively for clozapine-treated patients (black) and clozapine-naïve patients (grey) (see Example 1).

FIG. 1D illustrates density plots showing the distribution of serum immunoglobulin levels in patients receiving clozapine referred for Immunology assessment (light grey left-most curve, n=13) following removal of 4 patients (n=2 with haematological malignancy and n=2 previously included within the inventor's recent case-control study (Ponsford et al., 2018b)). Serum immunoglobulin distributions for clozapine-treated (mid-grey middle curve, n=94) and clozapine-naive (dark grey right-most curve, n=98) are also shown for comparison [adapted from (Ponsford et al., 2018b)]. Dotted lines represent the 5th and 95th percentiles for healthy adults (see Example 1). A leftward shift in the distribution curves of total immunoglobulin is observed in patients on clozapine for each of IgG, IgA and IgM compared to clozapine naive patients; this finding was particularly marked for clozapine referred patients.

FIG. 2. shows the effect of duration of clozapine use on serum IgG levels (see Example 1).

FIG. 3A. shows the number of class switched memory B cells (CSMB) (CD27+/IgM−/IgD−, expressed as a percentage of total CD19+ cells) in healthy controls, in patients taking clozapine referred to clinic and in patients with common variable immunodeficiency disorder (CVID) (see Example 1).

FIG. 3B. shows B cell subsets, expressed as a percentage of total CD19⁺ cells, in patients with schizophrenia with a history of clozapine therapy referred to clinic (numbers as shown), common variable immunodeficiency (CVID, n=26) and healthy controls (n=17). B-cell subsets gated on CD19⁺ cells and defined as follows: Naïve B-cells (CD27⁻IgD⁺IgM⁺), Marginal Zone-like B-cells (CD27⁺IgD⁺IgM⁺), Class-switched Memory B-cells (CD27⁺IgD⁻IgM⁻), and Plasmablasts (CD19⁺CD27^{Hi}IgD⁻). Non-parametric Mann-Whitney testing performed for non-normally distributed data, *p<0.05, **p<0.01, ***p<0.001, ****p<0.0001 (see Example 1).

FIG. 4A. shows the number of plasmablasts (CD38+++/IgM−, expressed as a percentage of total CD19+ cells) in healthy controls, in patients taking clozapine referred to clinic and in patients with common variable immunodeficiency disorder (CVID) (see Example 1).

FIG. 4B. illustrates vaccine specific-IgG response assessment (see Example 1).

FIG. 5. shows gradual recovery of serum IgG post-discontinuation of clozapine from 3.5 to 5.95 g/L over three years. LLN=lower limit of normal (see Example 1).

FIG. 6A-C. shows interim data findings on the levels of circulating IgG, IgA and IgM in patients on non-clozapine antipsychotics (‘control’, left) versus clozapine (right). Mean±SEM (see Example 2).

FIG. 7. shows interim data findings on peripheral blood levels of pneumococcal-specific IgG in patients on non-clozapine antipsychotics (‘control’, left) versus clozapine (right). Mean±SEM (see Example 2).

FIG. 8A-B. shows interim data findings on peripheral blood levels of B cells (CD19⁺) in patients on non-clozapine antipsychotics (‘control’, left) versus clozapine (right), expressed as absolute levels and as a percentage of lymphocytes (%, i.e. of T+B+NK cells). Mean±SEM (see Example 2).

FIG. 9A-C. shows interim data findings on peripheral blood levels of naive B cells (CD19⁺/CD27⁻) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 10A-C. shows interim data findings on peripheral blood levels of memory B cells (CD19⁺/CD27⁺) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 11A-C. shows interim data findings on peripheral blood levels of class switched (CS) memory B cells (CD27⁺/IgM⁻/IgD⁻) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 12A-C. shows interim data findings on peripheral blood levels of IgM high IgD low (CD27⁺/IgM⁺⁺/IgD⁻) memory B cells, i.e. post-germinal centre IgM only B cells, in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 13A-C. shows interim data findings on peripheral blood levels of transitional B cells (IgM⁺⁺/CD38⁺⁺) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 14A-C. shows interim data findings on peripheral blood levels of marginal zone (MZ) B cells (CD27⁺/IgD⁺/IgM⁺) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 15A-C. shows interim data findings on peripheral blood levels of plasmablasts in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 16. shows the body weight growth curve of WT mice in response to clozapine at different doses versus haloperidol and vehicle controls. Mean±SEM (see Example 3).

FIG. 17. shows body weight comparisons of WT mice at days 3, 12 and 21 of treatment. Mean±SEM (see Example 3).

FIG. 18. shows the impact of clozapine versus haloperidol and vehicle control on overall B cell content and pre-pro B cell and pro B cell precursors in bone marrow of WT mice. Mean±SEM (see Example 3).

FIG. 19. shows the impact of clozapine versus haloperidol and vehicle control on pre-B cells, proliferating B cells and immature B cell precursors in bone marrow of WT mice. Mean±SEM (see Example 3).

FIG. 20. shows the impact of clozapine versus haloperidol and vehicle control on class-switched memory B cells, plasmablasts and long-lived plasma cells in bone marrow of WT mice. Mean±SEM (see Example 3).

FIG. 21. shows the impact of clozapine versus haloperidol and vehicle control on overall B cells, T cells, other cell populations (TCR-β⁻/B220⁻) and activated T cells in spleen of WT mice. Mean±SEM (see Example 3).

FIG. 22. shows the impact of clozapine versus haloperidol and vehicle control on transitional (T1 and T2), follicular, marginal zone (MZ) and germinal centre (GC) B cells in spleen of WT mice. Mean±SEM (see Example 3).

FIG. 23. shows the impact of clozapine versus haloperidol and vehicle control on B cell subpopulations and T cells in the mesenteric lymph nodes (MLN) of WT mice. Mean±SEM. T1 and T2, transitional type 1 and type 2 B cells, respectively. MZ, marginal zone. GC, germinal centre (see Example 3).

FIG. 24. shows the impact of clozapine versus haloperidol and vehicle control on circulating immunoglobulins in WT mice. Mean±SEM (see Example 3).

FIG. 25. shows impact of clozapine on day of clinical onset of CIA. Mean±SEM (see Example 4).

FIG. 26. shows impact of clozapine on incidence of CIA (see Example 4).

FIG. 27. shows the impact of clozapine on the severity of CIA, judged by clinical score and thickness of first affected paw, in mice dosed from day 1 post-immunisation. Mean±SEM (see Example 4).

FIG. 28. shows the impact of clozapine on the severity of CIA, judged by number of affected paws by day of treatment with clozapine (day 15, D15 or day 1, D1) post-immunisation. Mean±SEM (see Example 4).

FIG. 29. shows the impact of clozapine versus control on B220⁺ (i.e. CD45⁺) cells in spleen and local lymph node of CIA mice. Mean±SEM (see Example 4).

FIG. 30. shows the impact of clozapine versus control on plasma cells (PC) in spleen and local lymph node of CIA mice. Mean±SEM (see Example 4).

FIG. 31. shows the impact of clozapine versus control on germinal centre (GC) B cells (B220⁺/IgD⁻/Fas⁺/GL7⁺) in spleen and local lymph node of CIA mice. Mean±SEM (see Example 4).

FIG. 32. shows the impact of clozapine versus control on expression of GL7 on germinal centre (GC) B cells (B220⁺/IgD⁻/Fas⁺/GL7⁺) in spleen and local lymph node of CIA mice. MFI, mean fluorescent intensity. Mean±SEM (see Example 4).

FIG. 33. shows the impact of clozapine versus control on peripheral blood anti-collagen IgG1 and IgG2a antibody levels of CIA mice (see Example 4).

FIG. 34. shows the impact of clozapine versus control on germinal centre resident T follicular helper cells (CD4⁺ PD1⁺) in spleen and local lymph node of CIA mice. Mean±SEM (see Example 4).

FIG. 35. shows the impact of clozapine versus control on expression of PD1 on germinal centre resident T follicular helper cells (CD4⁺ PD1⁺) in spleen and local lymph node of CIA mice. MFI, mean fluorescent intensity. Mean±SEM (see Example 4).

FIG. 36. shows the impact of clozapine versus control on expression of CXCR5 on germinal centre resident T follicular helper cells (CD4⁺ PD1⁺) in spleen and local lymph node of CIA mice. MFI, mean fluorescent intensity. Mean±SEM (see Example 4).

FIG. 37. shows the impact of clozapine versus control on expression of CCR7 on germinal centre resident T follicular helper cells (CD4⁺ PD1⁺) in spleen and local lymph node of CIA mice. MFI, mean fluorescent intensity. Mean±SEM (see Example 4).

FIG. 38. shows protocol schematic for in vitro generation/differentiation of human plasma cells (see Example 5).

FIG. 39. shows a schematic of the trial illustrating clozapine uptitration period followed by administration of typhoid vaccine (Typhim Vi) by injection (arrow) and then ongoing dosing with clozapine. Control cohort (vaccine only, no clozapine) and optional cohort (dose to be selected guided by findings from dose 1 and dose 3) (see Example 6).

### DETAILED DESCRIPTION OF THE INVENTION

The present invention also provides a method of treatment or prevention of a pathogenic IgE driven B cell disease in a subject by administering to said subject an effective amount of a compound selected from clozapine, norclozapine and prodrugs thereof and pharmaceutically acceptable salts and solvates thereof, in particular, wherein said compound causes mature B cells to be inhibited in said subject.

The present invention also provides use of a compound selected from clozapine, norclozapine and prodrugs thereof and pharmaceutically acceptable salts and solvates thereof in the manufacture of a medicament for the treatment or prevention of a pathogenic IgE driven B cell disease in a subject, in particular, wherein said compound causes mature B cells to be inhibited in said subject.

Clozapine or norclozapine may optionally be utilised in the form of a pharmaceutically acceptable salt and/or solvate and/or prodrug. In one embodiment of the invention clozapine or norclozapine is utilised in the form of a pharmaceutically acceptable salt. In a further embodiment of the invention clozapine or norclozapine is utilised in the form of a pharmaceutically acceptable solvate. In a further embodiment of the invention clozapine or norclozapine is not in the form of a salt or solvate. In a further embodiment of the invention clozapine or norclozapine is utilised in the form of a prodrug. In a further embodiment of the invention clozapine or norclozapine is not utilised in the form of a prodrug.

The term “pathogenic IgE driven B cell disease” includes B cell mediated disease, especially inflammatory disease, which involves exogenous antigens causing abnormally high and pathogenic IgE levels as a principal mechanism.

The range of exogenous antigens for pathogenic IgE driven B cell diseases include neutrophils (Churg-Strauss vasculitis) and pollen antigens (allergic rhinitis, allergic eye disease and atopic asthma, although there may be other causes).

Exemplary pathogenic IgE driven B cell diseases may be the lung related disease atopic asthma. Alternatively, the disease may be the skin related diseases atopic dermatitis and chronic non-autoimmune urticaria. Alternatively, the disease may be the neurological related disease Churg-Strauss vasculitis. Alternatively, the disease may be the nasal related disease allergic rhinitis. Alternatively, the disease may be the eye related disease allergic eye disease. Alternatively, the disease may be the oesophagus related disease eosinophilic esophagitis.

References highlighting the role of B cells and pathogenic IgE antibodies in the aforementioned diseases include:

### Eosinophilic Oesophagitis (EO)

EO is a chronic allergen-driven immune mediated disorder characterised pathologically by prominent eosinophilic infiltration (Chen and Kao, 2017). An important role for T helper type 2 cells (Th2) has been identified in response to allergens and associated production of IL-4, IL-5 and IL-13, with pathogenesis thought to be driven by a combination of IgE-mediated and non-IgE-mediated mechanisms (Weinbrand-Goichberg et al., 2013). Eosinophils promote inflammation, activate smooth muscle and induce mast and basophil cell degranulation (Chen and Kao, 2017).

Oesophageal biopsies from patients with EO reveal increased density of B cells and IgE-bound mast cells versus controls, with a positive correlation between CD20+ B cell density and mast cells (Vicario et al., 2010). Notably, an upregulation in expression of IgE heavy chain and mature IgE mRNA has been identified with evidence of local class-switch recombination (CSR) to IgE provided by detection of germline transcripts for ε, λ and γ4, in addition to expression of AID catalysing the initial step of CSR (Vicario et al., 2010). These findings indicate active B cell recruitment in EO, together with local CSR and mature IgE production in both ‘atopic’ and ‘non atopic’ individuals, indicating a clear local antibody response in EO (Vicario et al., 2010). Other studies have also identified increases in IgG subclasses, IgA and IgM in EO, particularly IgG4, the latter correlating with oesophageal eosinophil counts, histology and stage of disease (Rosenberg et al., 2018). Immune complex formation and IgG4⁺ plasma cells have been noted in the deep lamina propria (Clayton et al., 2014). Importantly, IgG4-switched B cells can switch to IgE but the reverse does not occur due to genetic deletion during the process of CSR to IgE. Notably the transition to a plasma cell phenotype occurs early with IgE B cells and significantly more so than with IgG-switched B cells (Aalberse et al., 2016).

A role for IgE is further suggested by the observation of IgE-bearing cells including mast cells in EO (Straumann et al., 2001). Notably, cells expressing the high affinity receptor for IgE, FcεRI, are present in large numbers in the oesophageal epithelium of patients with EO, suggesting this receptor to be important in IgE-mediated activation of immune cells in EO (Yen et al., 2010).

EO is often associated with IgE sensitisation to allergens in food in children and plant/aero allergens in adults. Serum IgE levels are also often significantly elevated in patients with EO, consistent with the presence of IgE-producing long-lived plasma cells (Aalberse et al., 2016), which together with specific IgE antibodies suggest a contribution of IgE to the pathogenesis (Straumann et al., 2001). Notably food-specific IgE antibodies predict oesophageal eosinophilia in children (Erwin et al., 2017). Active oesophagitis in EO is associated with elevated oesophageal levels of plasma cells (Mohammad et al., 2018).

Notably, in addition to immunoglobulin targeting exogenous antigens, recent data suggest the presence of autoantibodies in EO, specifically anti-NC16A which appear to correlate with histological response (Dellon et al., 2018).

Supporting a pathogenic role for IgE in EO, a pilot study of anti-IgE treatment using omalizumab demonstrated clinical improvement in a proportion of patients, accompanied by lower tissue IgE levels, tryptase positive cells and eosinophils (Loizou et al., 2015).

Atopic Asthma (Extrinsic, Early-Onset or Allergic Asthma) Atopic asthma I associated with atopy (allergic rhinitis and atopic eczema) and thought to be driven substantially by a type 2 helper T cell (T_{H}2) response to promote an early IgE-mediated (i.e. type 1) hypersensitivity reaction, together with IL-5 promoted activation of eosinophils.

Notably T_{H}2 cells secrete high levels of IL-4 and IL-13 which promote IgE class-switching by B cells. Subsequently, IgE memory B cells can differentiate into plasma cells to produce specific IgE that can bind to its high affinity receptor, FcεRI, on target cells such as mast cells and basophils (Palomares et al., 2017). Binding of antigen-specific IgE to FcεRI on mast cells is critical in sensitising these cells to release mast cell mediators in a specific manner. In addition, immune complexes formed from antigen-IgE can bind to CD23 on B cells or FcεRI to further amplify IgE-associated immune responses (Galli and Tsai, 2012). Importantly, bronchial epithelial cells from some patients with asthma, but not healthy controls, express FcεRI, are capable of fixing IgE and functional in terms of eicosanoid release (Campbell et al., 1998).

Importantly, class switch recombination from IgM/IgG/IgA to IgE can occur locally in bronchial tissue in asthma, resulting in clonal selection and affinity maturation of IgE-producing B cells to release IgE locally (Takhar et al., 2007). Patients with allergic asthma exhibit highly elevated levels of IgE⁺CD19⁺ B cells in the airways compared to healthy controls and ‘allergic’ controls, as well as increased IgE⁺ memory B cells and IgE⁺ plasma cells (Oliveria et al., 2017). Notably, the frequency of IgE+ B cells corelates positively with airway levels of eosinophils, IgE and BAFF, findings consistent with local maturation and proliferation of IgE⁺ B cells in the airways of patients with allergic asthma (Oliveria et al., 2017) to drive the disease process through their production of IgE and potent antigen-presentation function (Wypych et al., 2018). Furthermore, tissue resident memory B cells have also been identified in the airways of a mouse model of allergic asthma, providing a resident B cell population that can be rapidly locally activated in response to allergen/antigen re-exposure (Turner et al., 2017).

Children with asthma and/or atopy show evidence of expanded IgE⁺ plasmablasts in addition to IgE memory cells and T_{H}2 cells; notably plasmablast numbers positively correlate with frequency of circulating T_{H}2 cells (Heeringa et al., 2018).

Substantiating a key role for B cells in the pathogenesis of atopic asthma, B cell depletion using anti-CD20 before house dust mite (HDM) challenge in HDM sensitised mice markedly reduces the allergic response, with reduced CD4⁺CD44⁺ T cells, eosinophils and neutrophils in lung immune infiltrates consistent with a lower T_{H}2 response (Wypych et al., 2018). B cells thus play a critical role in amplifying T_{H}2 responses in vivo promote the allergic response, this is likely to in part reflect their ability to efficiently present antigen (Wypych et al., 2018).

Substantiating a role for IgE in atopic/allergic asthma, administration of monoclonal antibody therapy targeting FcεRI (thereby inhibiting the binding of endogenous IgE to mast and other effector cells without stimulating mast cell activation) to allergic asthmatic patients suppresses early and late phase responses to inhaled allergen, associated with lowering of serum IgE, blunting of sensitivity to inhaled allergen and attenuation of the fall in respiratory capacity associated with allergen inhalation (Fahy et al., 1997). Further evidence for the critical pathogenic role of IgE in persistent asthma comes from a trial of omalizumab, a humanised monoclonal anti-IgE antibody, in inner city children and young adults. This demonstrated a significant reduction in burden of asthma symptoms, frequency of exacerbations, particularly seasonal peaks, despite a lower requirement for inhaled glucocorticoid and β-agonist bronchodilator therapy (Busse et al., 2011). Specific extracorporeal immunoadsorption of IgE has also demonstrated efficacy in reducing allergen-specific basophil sensitivity and clinical symptoms during pollen season in patients with allergic asthma (Lupinek et al., 2017). Unlike omalizumab administration, this approach while invasive is not limited by threshold levels of IgE (Lupinek et al., 2017).

### Atopic Dermatitis (AD; Atopic Eczema)

AD is a chronic inflammatory skin disorder characterised by pruritic eczematous skin lesions. It is associated with other stopic diseases (asthma and allergic rhinitis), with shared aspects of pathophysiology, in particular a propensity to form IgE antibodies and sensitisation to exogenous triggers (Zheng et al., 2011). AD is considered a biphasic T cell-mediated disorder with a T_{H}2 to T_{H}1 switch promoting chronicity, in addition to a significant disease component driven by B cell derived IgE (Furue et al., 2017).

Notably ^{˜}80-90% of patients with AD feature raised serum IgE, with elevated levels correlating with IgE autoreactivity against multiple antigens (Furue et al., 2017). The enhanced levels of IgE are thought to primarily reflect greater numbers of IgE antibody-producing cells (Thomas et al., 1995).

Patients with AD display IgE autoantibodies against keratinocyte proteins, particularly in severe cases (Altrichter et al., 2008). The correlation of autoreactive IgE in AD with clinical severity and absence in other skin disorders supports a role for IgE-mediated autoreactivity in disease pathogenesis (Navarrete-Dechent et al., 2016). Notably AD also occurs in association with other autoimmune diseases, e.g. vitiligo (Mohan and Silverberg, 2015), with a proportion of patients with severe facial rashes exhibiting ANA positivity (Higashi et al., 2009), suggest more generalised humoral immune dysregulation in AD. Autoantibodies described include those targeting SART-1, cytokeratin type II, hMnSOD and BCL7B amongst others (Navarrete-Dechent et al., 2016). Clinical severity scores correlate strongly with some of these specific IgE autoantibodies (Schmid-Grendelmeier et al., 2005). The allergenicity of this antigen is further supported by its ability to induce T-cell proliferation and positive immediate responses to skin challenge (Schmid-Grendelmeier et al., 2005).

Further supporting a role for B cells in AD, analysis of peripheral lymphocyte subsets has revealed B cell alterations particularly in children with AD, with positive correlations between activated B cells and memory T cell levels (Czarnowicki et al., 2017). Furthermore, peripheral blood analysis indicates that early AD is characterised by aberrant B cell maturation and reveals a positive correlation between memory B cells and T_{H}1 and T_{H}2 cells in AD (Czarnowicki et al., 2017). Importantly, in children with AD IgE sensitisation is seen to cluster with total IgE levels, switched memory B cells and T_{H}1/T_{H}2 cells, with evidence of accelerated B cell development that would support IgE class switching (Czarnowicki et al., 2017).

In adults, AD is associated with an increase in circulating transitional B cells, chronically activated memory B cells, plasmablasts and IgE memory B cells (Czarnowicki et al., 2016). Notably circulating cell expression of CD23, the low-affinity receptor for the Fc region of IgE (FcεRII), is increased in AD and correlates with AD clinical severity (Czarnowicki et al., 2016); this observation is notable given the role of CD23 to promote IgE synthesis/responses (Pene, 1989).

Supporting a major role for B cells in the pathogenesis of AD, B cell depletion using rituximab results in substantial clinical improvement (severity/area affected), in conjunction with improvements in histology (reduced B and T cell infiltration), IL-5/IL-13 and some reduction in total IgE (Simon et al., 2008). Notably, despite near complete depletion of circulating B cells, those in the skin were less substantially reduced (by ^{˜}50%), with plasma cells also evident in skin samples both before and after therapy (Simon et al., 2008). Furthermore, sequential treatment of severe refractory AD using anti-IgE (omalizumab) followed by B cell depletion (rituximab) has reported dramatic clinical responses in conjunction with lowering of serum IgE and peripheral blood B cell levels (Sanchez-Ramon et al., 2013). Further supporting a role for pathogenic IgE immunoglobulin in driving AD, repeated IgE immunoadsorption in patients with AD and increased serum IgE results in significant clinical improvement together with lowering of IgE (Daeschlein et al., 2015).

Churg-Strauss Syndrome (Churg-Strauss Vasculitis; Eosinophilic Granulomatosis with Polyangiitis; CSS/EGPA)

Churg-Strauss syndrome, also known as eosinophilic granulomatosis with polyangiitis (EGPA), is a small-medium vessel systemic necrotising vasculitis, part of the clinical spectrum of ANCA-associated vasculitis (ANCA, antineutrophil cytoplasm antibody positive in ^{˜}40) and associated with severe adult-onset asthma, sinusitis and blood/tissue eosinophilia (Groh et al., 2015).

The pathogenesis of CSS involves T cells (particularly excessive T_{H}2 responses but also involvement of T_{H}1 and reduced regulatory T cells), activated tissue eosinophils and B cells with a humoral response (Greco et al., 2015). ANCAs are directly pathogenic primarily target myeloperoxidase (MPO) and proteinase 3, with the former characteristic for CCS. ANCA result in neutrophil activation and degranulation leading to cytokine, cytolytic enzyme and ROS release through binding of ANCA-specific antigens and, via their Fc region, the Fcγ receptor on neutrophils (Nakazawa et al., 2019).

Supporting a role for IgE in pathogenesis of CSS, mice subjected to a cutaneous reverse passive Arthus reaction (using IgE) to provide an IgE-immune complex challenge develop cutaneous eosinophilis vasculitis reminiscent of CSS (Ishii et al., 2009). Notably eosinophil infiltration in this model is strikingly specific for IgE-mediated immune complex challenge and barely seen with IgG antibody injection (Ishii et al., 2009). Evidence also exists to support pathogenicity of IgE in CSS via immune complex formation and activation of complement (Manger et al., 1985).

Patients with CSS who exhibit active disease and frequent relapse show increased levels of activated B cells and reduced levels of circulating T regulatory cells (Tsurikisawa et al., 2013). Patients with CSS also demonstrate a cellular milieu conducive to plasma cell differentiation and antibody-mediated responses through an increase in IL-21 secreting T helper cells, specifically in ANCA positive patients (Abdulahad et al., 2013).

Supporting a key role for B cells and their autoantibodies in the pathogenesis of CCS, B cell depletion using rituximab is clinically effective in inducing remission or partial responses and lowering of requirement for corticosteroid therapy; notably levels of baseline ANCA associate with higher levels of remission (Mohammad et al., 2016). These findings have been confirmed in another clinical study of refractory patients with CSS, with rituximab lowering levels of IgE, CRP and eosinophils in conjunction with inducing remission (Thiel et al., 2017). There is also evidence for a corticosteroid-sparing effect of targeting IgE using omalizumab in refractory/relapsing CCS (Jachiet et al., 2016).

### Allergic Rhinitis (AR)

AR is a common and chronic IgE-mediated inflammatory nasal disorder frequently associated with other atopic features (asthma and atopic dermatitis). Exposure to specific allergens promotes allergen-specific IgE production which can then bind to target cells (e.g. mast cells and basophils) via the high affinity receptor, FcεRI (Wise et al., 2018). In turn, nasal mast cells from patients with AR exhibit upregulation of FcεRI expression and increased cell-bound IgE correlating with serum IgE levels; these cells can also induce IgE production by B cells indicating a feed-forward IgE− FcεRI-mast cell axis critically dependent on pathogenic IGE that can perpetuate AR (Pawankar and Ra, 1998).

Nasal mucosal B cells are over 1000-fold more frequent than in peripheral blood in AR and produce IgE following allergen exposure (Coker et al., 2003; Takhar et al., 2005). There is evidence supporting local class switch recombination in nasal mucosa of patients with AR (Cameron et al., 2000), suggesting that tissue resident/local B cells under Ig isotype switching to IgE in the context of local immune responses to allergen (Cameron et al., 2003). Both IgE+ B cells and IgE+ plasma cells are enriched in the nasal mucosa of patients with AR (KleinJan et al., 2000).

Substantiating a central role for IgE in AR, anti-IgE therapy using omalizumab is clinically effective in patients with AR, also inhibiting seasonal associated allergen-induced increases in tissue/blood eosinophils (Holgate et al., 2005; Tsabouri et al., 2014).

### Allergic Eye Disease

Seasonal and perennial allergic conjunctivitis are the commonest forms of allergic eye disease and are associated with other atopic diseases with mechanisms similar to those outlined, including an important role for B cell derived IgE. Supporting this, anti-IgE therapy using omalizumab has shown efficacy in atopic individuals with coexisting eye disease (Kopp et al., 2009).

### Chronic Non-Autoimmune Urticaria (Chronic Spontaneous Urticaria, CSU)

Urticaria is a common, mast cell-driven disease, and can be classified as acute or chronic; chronic non-autoimmune urticaria can itself be classified as chronic spontaneous urticaria (CSU) and chronic inducible urticaria (Radonjic-Hoesli et al., 2018). Although there are no obvious external triggers in CSU, and most patients have an autoimmune cause, there is a significant proportion of patients that do not have an autoimmune disease. In these, IgE binding to FcεRI on mast cells without cross-linking is thought to promote survival and proliferation of mast cells, decrease the threshold for mast cell mediator release (Chang et al., 2015). Consistent with this and a pathogenic role for B cell-derived IgE in this condition, to date, there have been 2 phase II and 4 phase III randomized, placebo-controlled clinical trials that have convincingly established that IgE depletion using anti-IgE therapy with omalizumab is efficacious and safe for treating CSU that is refractory to the current standard care (Chang et al., 2015).

Thus, in an embodiment, the invention provides (i) a compound selected from clozapine, norclozapine and prodrugs thereof and pharmaceutically acceptable salts and solvates thereof for use in the treatment or prevention of a pathogenic IgE driven B cell disease in a subject and (ii) a method of treatment or prevention of a pathogenic IgE driven B cell disease in a subject by administering to said subject an effective amount of a compound selected from clozapine, norclozapine and prodrugs thereof and pharmaceutically acceptable salts and solvates thereof wherein in the case of (i) and (ii) the pathogenic IgE driven B cell disease is a disease selected from the group consisting of atopic asthma, atopic dermatitis, chronic non-autoimmune urticaria, Churg-Strauss vasculitis, allergic rhinitis and allergic eye disease preferably atopic dermatitis, atopic asthma, allergic rhinitis and eosinophilic esophagitis.

Preferably the disease is selected from atopic dermatitis, atopic asthma and allergic rhinitis.

Clozapine is associated with high levels of CNS penetration which could prove to be a valuable property in treating some of these diseases (Michel et al., 2015).

In certain diseases, such as atopic dermatitis, there may also be a T cell component that contributes towards the pathology of the disease. This arises because B cells act as professional antigen-presenting cells for T cells (their importance is increased also due to their sheer numbers). B cells secrete significant amounts of cytokines that impact T cells. B-T interaction is involved in responses to T dependent protein antigens and class switching. Therefore, clozapine and norclozapine are expected to have an effect on T cells due to their effect on reducing B cell numbers.

Suitably the compound selected from clozapine, norclozapine and prodrugs thereof inhibits mature B cells, especially CSMBs and plasmablasts, particularly CSMBs. “Inhibit” means reduce the number and/or activity of said cells. Thus, suitably clozapine or norclozapine reduces the number of CSMBs and plasmablasts, particularly CSMBs.

In an embodiment, the compound selected from clozapine, norclozapine and prodrugs thereof has the effect of decreasing CD19 (+) B cells and/or CD19 (−) B plasma cells.

The term “treatment” means the alleviation of disease or symptoms of disease. The term “prevention” means the prevention of disease or symptoms of disease. Treatment includes treatment alone or in conjunction with other therapies. Treatment embraces treatment leading to improvement of the disease or its symptoms or slowing of the rate of progression of the disease or its symptoms. Treatment includes prevention of relapse.

The term “effective amount” refers to an amount effective, at dosages and for periods of time necessary, to achieve the desired therapeutic result, in which any toxic or detrimental effects of the pharmacological agent are outweighed by the therapeutically beneficial effects. It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation. Example dosages are discussed below.

As used herein, a “subject” is any mammal, including but not limited to humans, non-human primates, farm animals such as cattle, sheep, pigs, goats and horses; domestic animals such as cats, dogs, rabbits; laboratory animals such as mice, rats and guinea pigs that exhibit at least one symptom associated with a disease, have been diagnosed with a disease, or are at risk for developing a disease. The term does not denote a particular age or sex. Suitably the subject is a human subject.

It will be appreciated that for use in medicine the salts of clozapine and norclozapine should be pharmaceutically acceptable. Suitable pharmaceutically acceptable salts will be apparent to those skilled in the art. Pharmaceutically acceptable salts include those described by Berge, Bighley and Monkhouse J. Pharm. Sci. (1977) 66, pp 1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of clozapine and are included within the scope of this invention.

Clozapine or norclozapine may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water).

A “prodrug”, such as an N-acylated derivative (amide) (e.g. an N-acylated derivative of norclozapine) is a compound which upon administration to the recipient is capable of providing (directly or indirectly) clozapine or an active metabolite or residue thereof. Other such examples of suitable prodrugs include alkylated derivatives of norclozapine other than clozapine itself.

Isotopically-labelled compounds which are identical to clozapine or norclozapine but for the fact that one or more atoms are replaced by an atom having an atomic mass or mass number different from the atomic mass or mass number most commonly found in nature, or in which the proportion of an atom having an atomic mass or mass number found less commonly in nature has been increased (the latter concept being referred to as “isotopic enrichment”) are also contemplated for the uses and method of the invention. Examples of isotopes that can be incorporated into clozapine or norclozapine include isotopes of hydrogen, carbon, nitrogen, oxygen, fluorine, iodine and chlorine such as ²H (deuterium), ³H, ¹¹C, ¹³C, ¹⁴C, ¹⁸F, ¹²³I or ¹²⁵I, which may be naturally occurring or non-naturally occurring isotopes.

Clozapine or norclozapine and pharmaceutically acceptable salts of clozapine or norclozapine that contain the aforementioned isotopes and/or other isotopes of other atoms are contemplated for use for the uses and method of the present invention. Isotopically labelled clozapine or norclozapine, for example clozapine or norclozapine into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

Since clozapine or norclozapine are intended for use in pharmaceutical compositions it will readily be understood that it is preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

In general, clozapine or norclozapine may be made according to the organic synthesis techniques known to those skilled in this field (as described in, for example, U.S. Pat. No. 3,539,573.

A compound selected from clozapine, norclozapine and prodrugs thereof and pharmaceutically acceptable salts and solvates thereof for use in therapy is usually administered as a pharmaceutical composition. Also provided is a pharmaceutical composition comprising clozapine or norclozapine, or a pharmaceutically acceptable salt and/or solvate and/or prodrug thereof and a pharmaceutically acceptable diluent or carrier. Said composition is provided for use in the treatment or prevention of a pathogenic IgE driven B cell disease in a subject wherein said compound causes mature B cells to be inhibited in said subject.

A compound selected from clozapine, norclozapine and prodrugs thereof and pharmaceutically acceptable salts and solvates thereof may be administered by any convenient method, e.g. by oral, parenteral, buccal, sublingual, nasal, rectal or transdermal administration, and the pharmaceutical compositions adapted accordingly. Other possible routes of administration include intratympanic and intracochlear. Suitably, a compound selected from clozapine, norclozapine and prodrugs thereof and pharmaceutically acceptable salts and solvates thereof are administered orally.

A compound selected from clozapine, norclozapine and prodrugs thereof and pharmaceutically acceptable salts and solvates thereof which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal or pulmonary administration may conveniently be formulated as aerosols, sprays, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal or pulmonary inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatine and glycerine.

Compositions for rectal administration are conveniently in the form of suppositories containing a conventional suppository base such as cocoa butter.

Compositions suitable for topical administration to the skin include ointments, gels and patches.

In one embodiment the composition is in unit dose form such as a tablet, capsule or ampoule.

Compositions may be prepared with an immediate release profile upon administration (i.e. upon ingestion in the case of an oral composition) or with a sustained or delayed release profile upon administration.

For example, a composition intended to provide constant release of clozapine over 24 hours is described in WO2006/059194 the contents of which are herein incorporated in their entirety.

The composition may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, of the active material (i.e. clozapine or norclozapine), depending on the method of administration. The composition may contain from 50 mg to 1000 mg, for example from 100 mg to 400 mg of the carrier, depending on the method of administration. The dose of clozapine or norclozapine used in the treatment or prevention of the aforementioned diseases will vary in the usual way with the seriousness of the diseases, the weight of the sufferer, and other similar factors. However, as a general guide suitable unit doses of clozapine as free base may be 0.05 to 1000 mg, more suitably 1.0 to 500 mg, and such unit doses may be administered more than once a day, for example two or three a day. Such therapy may extend for a number of weeks or months.

A compound selected from clozapine, norclozapine and prodrugs thereof and pharmaceutically acceptable salts and solvates thereof may be administered in combination with another therapeutic agent for the treatment of pathogenic IgE driven B cell diseases, such as those that inhibit B cells or B cell-T cell interactions. Other therapeutic agents include for example: anti-TNFα agents (such as anti-TNFα antibodies e.g. infliximab or adalumumab), calcineurin inhibitors (such as tacrolimus or cyclosporine), antiproliferative agents (such as mycophenolate e.g. as mofetil or sodium, or azathioprine), general anti-inflammatories (such as hydroxychloroquine or NSAIDS such as ketoprofen and colchicine), mTOR inhibitors (such as sirolimus), steroids (such as prednisone), anti-CD80/CD86 agents (such as abatacept), anti-CD-20 agents (such as anti-CD-20 antibodies e.g. rituximab), anti-BAFF agents (such as anti-BAFF antibodies e.g. tabalumab or belimumab, or atacicept), immunosuppressants (such as methotrexate or cyclophosphamide), anti-FcRn agents (e.g. anti-FcRn antibodies), anti-IgE antibodies (e.g. omalizumab) and other antibodies (such as ARGX-113, PRN-1008, SYNT-001, veltuzumab, ocrelizumab, ofatumumab, obinutuzumab, ublituximab, alemtuzumab, milatuzumab, epratuzumab and blinatumomab).

Other therapies that may be used in combination with the invention include non-pharmacological therapies such as intravenous immunoglobulin therapy (IVIg), subcutaneous immunoglobulin therapy (SCIg) eg facilitated subcutaneous immunoglobulin therapy, plasmapheresis and immunoabsorption.

Thus the invention provides a compound selected from clozapine, norclozapine and prodrugs thereof and pharmaceutically acceptable salts and solvates thereof for use in the treatment or prevention of a pathogenic IgE driven B cell disease in combination with a second or further therapeutic agent for the treatment or prevention of a pathogenic IgE driven B cell disease e.g. a substance selected from the group consisting of anti-TNFα agents (such as anti-TNFα antibodies e.g. infliximab or adalumumab), calcineurin inhibitors (such as tacrolimus or cyclosporine), antiproliferative agents (such as mycophenolate e.g. as mofetil or sodium, or and azathioprine), general anti-inflammatories (such as hydroxychloroquine and NSAIDS such as ketoprofen and colchicine), mTOR inhibitors (such as sirolimus), steroids (such as prednisone), anti-CD80/CD86 agents (such as abatacept), anti-CD-20 agents (such as anti-CD-20 antibodies e.g. rituximab). anti-BAFF agents (such as anti-BAFF antibodies e.g. tabalumab or belimumab, or atacicept), immunosuppressants (such as methotrexate or cyclophosphamide), anti-FcRn agents (e.g. anti-FcRn antibodies) and other antibodies (such as ARGX-113, PRN-1008, SYNT-001, veltuzumab, ocrelizumab, ofatumumab, obinutuzumab, ublituximab, alemtuzumab, milatuzumab, epratuzumab and blinatumomab).

When a compound selected from clozapine, norclozapine and prodrugs thereof and pharmaceutically acceptable salts and solvates thereof is used in combination with other therapeutic agents, the compounds may be administered separately, sequentially or simultaneously by any convenient route.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations. The individual components of combinations may also be administered separately, through the same or different routes. For example, a compound selected from clozapine, norclozapine and prodrugs thereof and pharmaceutically acceptable salts and solvates thereof and the other therapeutic agent may both be administered orally. Alternatively, a compound selected from clozapine, norclozapine and prodrugs thereof and pharmaceutically acceptable salts and solvates thereof may be administered orally and the other therapeutic agent via may be administered intravenously or subcutaneously.

Typically, a compound selected from clozapine, norclozapine and prodrugs thereof and pharmaceutically acceptable salts and solvates thereof is administered to a human.

### EXAMPLES

### Example 1

### First Observational Study on Human Patients on Anti-Psychotic Therapy

To assess a possible association between antibody deficiency and clozapine use the inventors undertook a cross-sectional case control study to compare the immunoglobulin levels and specific antibody levels (against Haemophilus B (Hib), Tetanus and Pneumococcus) in patients taking either clozapine or alternative antipsychotics.

### Method

Adults (>18 yrs) receiving either clozapine or non-clozapine antipsychotics were recruited during routine clinic visits to ten Community Mental Health Trust (CMHT) outpatient clinics in Cardiff & Vale and Cwm Taf Health Boards by specialist research officers between November 2013 and December 2016 (Table 1). Following consent, participants completed a short lifestyle, drug history and infection questionnaire followed by blood sampling. Where required, drug histories were confirmed with the patient's General Practice records. Formal psychiatric diagnoses and antipsychotic medication use were confirmed using the medical notes, in line with other studies. Patients' admission rates were confirmed by electronic review for the 12-month period prior to recruitment. Patients with known possible causes of hypogammaglobulinemia including prior chemotherapy, carbamazepine, phenytoin, antimalarial agents, captopril, high-dose glucocorticoids, hematological malignancy and 22q11 deletion syndrome were excluded.

Clinical and immunological data from 13 patients taking clozapine, 11 of whom had been referred independently of the study for assessment in Immunology clinic, are presented in Table 3. Laboratory data on these, healthy controls and patients with common variable immunodeficiency (CVID) are shown in FIG. 3. The 11 independently referred patients were excluded from the overall study analysis.

Immunoglobulin levels (IgG, IgA and IgM) were assayed by nephelometry (Siemens BN2 Nephelometer; Siemens), serum electrophoresis (Sebia Capillarys 2; Sebia, Norcross, Ga., USA) and, where appropriate, serum immunofixation (Sebia Hydrasys; Sebia, Norcross, Ga., USA). Specific antibody titres against Haemophilus influenzae, Tetanus and Pneumococcal capsular polysaccharide were determined by ELISA (The Binding Site, Birmingham, UK). Lymphocyte subsets, naïve T cells and EUROclass B cell phenotyping were enumerated using a Beckman Coulter FC500 (Beckman Coulter, California, USA) flow cytometer. All testing was performed in the United Kingdom Accreditation Service (UKAS) accredited Immunology Laboratory at the University Hospital of Wales. Laboratory adult reference ranges for immunoglobulin levels used were, IgG 6-16 g/L, IgA 0.8-4 g/L, IgM 0.5-2 g/L.

Statistical analysis of the laboratory and clinical data was performed using Microsoft Excel and Graphpad Prism version 6.07 (Graphpad, San Diego, Calif., USA). Independent samples t-test were performed unless D'Agoustino & Pearson testing showed significant deviation from the Gaussian distribution, in which case the non-parametric Mann-Whitney test was used. All tests were two-tailed, using a significance level of p<0.05.

### Results

### Study Participants

A total of 291 patients taking clozapine and 280 clozapine-naïve patients were approached and 123 clozapine and 111 clozapine-naïve patients consented to the study (Table 1). Recruitment was stopped as per protocol when the target of 100 patients in each group had been achieved. There were small differences in gender with more males in the clozapine-treated group (53% versus 50%) and a lower mean age in the clozapine group (45 versus 50 years). These differences are unlikely to be relevant as there are no gender differences in the adult reference range for serum immunoglobulins and there is a male predominance in schizophrenia. Levels of smoking, diabetes, COPD/asthma, and alcohol intake were similar between the groups. More patients were admitted to hospital with infection in the clozapine group (0.12 vs 0.06 per patient year) and more took >5 courses of antibiotics per year compared with controls (5.3% vs 2%). The possible impact of a diagnosis of schizophrenia, medications and smoking as risk factors for antibody deficiency were assessed in a subgroup analysis (Table 2).

TABLE 1
Clozapine-treated and clozapine-naïve patient characteristics

| | Clozapine-Treated | Clozapine-Naive |
|---|---|---|
| Total screened | 291 | 280 |
| Declined, lacked capacity, or | 168 | 169 |
| unable to obtain blood sample | | |
| Initial Screening | 123 | 111 |
| Sex (M:F) | (81:42) | (56:55) |
| Mean age, years | 45.3 | 50.3 |
| (Range) | (22.0-78.0) | (21.6-78.0) |
| Post-exclusion | 94 | 98 |
| (% total screened) | (32%) | (35%) |
| Sex (M:F) | 64:30 | 54:44 |
| Mean age, years | 44.4 | 50.4 |
| (Range) | (22.0-78.0) | (21.6-78.0) |
| Primary Psychiatric Diagnosis | | |
| Schizophrenia | 87 | 58 |
| Schizoaffective | 1 | 5 |
| Bipolar | 0 | 11 |
| Psychosis | 0 | 15 |
| Depression | 0 | 3 |
| Personality Disorder | 2 | 2 |
| Anxiety disorder | 0 | 2 |
| Electronic record incomplete | 4 | 2 |
| Dual antipsychotic treatment | 30.9% | 11.2% |
| Duration antipsychotic use | 8.0 | 7.0 |
| (median, range), years | (0.1-20) | (0.1-44) |
| Current smoking (%) | 60.6% | 56.1% |
| Diabetes (%) | 20.2% | 17.3% |
| COPD/Asthma (%) | 13.8% | 16.3% |
| Alcohol intake mean | 5.3 (0-60) | 6.0 (0-68) |
| (units/week), range | | |
| Antibiotic courses per year | | |
| Nil courses | 61.7% | 63.3% |
| 1-5 courses | 33.0% | 34.7% |
| >5 courses | 5.3% | 2.0% |
| Admission frequency | | |
| in 12-month period | | |
| All cause | 21 (14 patients) | 14 (13 patients) |
| Infection-related | 15 (10 patients) | 7 (6 patients) |

### Effects of Clozapine on Antibody Levels

FIG. 1 A-C shows significantly reduced concentrations of all three immunoglobulin classes (IgG, IgA and IgM) in patients receiving clozapine, with a shift towards lower immunoglobulin levels in the distribution as a whole for each of IgG, IgA and IgM compared to the clozapine-naïve control group. The percentages of the 123 patients having immunoglobulin levels below the reference range were IgG 9.8% (p<0.0001), IgA 13.0% (p<0.0001) and IgM 38.2% (p<0.0001) compared with the 111 clozapine-naïve IgG 1.8%, IgA 0.0% and IgM 14.4%. Large percentages of both clozapine-treated and clozapine-naïve patients had specific antibody levels below the protective levels for HiB (51% and 56% less than 1 mcg/ml, (Orange et al., 2012)), Pneumococcus (54% and 56% less than 50 mg/L, (Chua et al., 2011)) and Tetanus (12% and 14% less than 0.1 IU/ml). The Pneumococcal IgA (31 U/ml vs 58.4 U/ml p<0.001) and IgM (58.5 U/ml vs 85.0 U/ml p<0.001) levels are significantly lower in clozapine-treated versus clozapine-naïve patients.

Subgroup analysis (Table 2) was undertaken to determine if the reductions in immunoglobulins were potentially explained by confounding factors including any other drugs, a diagnosis of schizophrenia and smoking. The assessment of the effect of excluding other secondary causes of antibody deficiency (plus small numbers where additional diagnoses were uncovered—Table 1) is shown in Column B. The number of patients excluded on the basis of taking anti-epileptic medications was higher in the clozapine-treated group and is likely to reflect the use of these agents for their mood stabilizing properties rather than as treatment for epilepsy.

TABLE 2
Immunoglobulin levels and specific antibody levels in sub-groups A-D

| | A | | B | | C | | D | |
|---|---|---|---|---|---|---|---|---|
| Medication: | Clozapine | Control | Clozapine | Control | Clozapine | Control | Clozapine | Control |
| Diagnosis: | All | All | All | All | Schizophrenia | | All | All |
| | | | | | diagnoses only | | | |
| Smoking: | All | All | All | All | All | All | Smokers only | |
| Possible | No | No | Yes | | Yes | | Yes | |
| secondary | | | | | | | | |
| causes | | | | | | | | |
| excluded | | | | | | | | |
| Sample size: | 123 | 111 | 94 | 98 | 87 | 58 | 57 | 55 |
| Serum IgG | 95% Cl: 0.89-2.32**** | | 95% Cl: 0.98 to | | 95% Cl: 0.92 to | | Non-Gaussian | |
| (Reference | | | 2.59**** | | 2.77*** | | distribution† | |
| range 6-16 g/L) | | | | | | | | |
| <3 | 0.8% | 0.0% | 1.1% | 0.0% | 1.2% | 0.0% | 1.8% | 0.0% |
| <4 | 1.6% | 0.0% | 1.1% | 0.0% | 1.2% | 0.0% | 1.8% | 0.0% |
| <5 | 3.3% | 0.0% | 2.1% | 0.0% | 2.3% | 0.0% | 1.8% | 0.0% |
| <6 | 9.8% | 1.8% | 8.4% | 1.0% | 9.2% | 1.7% | 8.8% | 1.8% |
| Serum IgA | 95% Cl: 0.55 to | | 95% Cl: 0.55 to | | 95% Cl: 0.59 to | | 95% Cl: 0.41 to | |
| (Reference | 1.01**** | | 1.05**** | | 1.19**** | | 1.04**** | |
| range 0.8-4.0 | | | | | | | | |
| g/L) | | | | | | | | |
| <0.5 | 1.6% | 0.0% | 2.1% | 0.0% | 2.3% | 0.0% | 3.5% | 0.0% |
| <0.6 | 2.4% | 0.0% | 2.1% | 0.0% | 3.5% | 00% | 3.5% | 0.0% |
| <0.7 | 6.5% | 0.0% | 6.4% | 0.0% | 6.9% | 0.0% | 3.5% | 0.0% |
| <0.8 | 13.0% | 0.0% | 13.8% | 0.0% | 14.9% | 0.0% | 10.5% | 0.0% |
| Serum IgM | Non-Gaussian | | 95% Cl: 0.10 to | | 95% Cl: 0.06 to | | 95% Cl: 0.02 to | |
| (Reference | distribution†††† | | 0.38*** | | 0.38** | | 0.39* | |
| range 0.5- | | | | | | | | |
| 1.9 g/L) | | | | | | | | |
| <0.2 | 8.1% | 0.0% | 5.3% | 0.0% | 5.8% | 0.0% | 1.78% | 0.0% |
| <0.3 | 16.3% | 2.7% | 12.8% | 3.1% | 12.6% | 5.2% | 12.3% | 1.8% |
| <0.4 | 29.3% | 8.1% | 26.6% | 8.2% | 27.6% | 6.9% | 26.3% | 9.1% |
| <0.5 | 38.2% | 14.4% | 34.0% | 15.3% | 35.6% | 13.8% | 33.3% | 18.2% |
| IgG- | 95% Cl: −8.25 to | | 95% Cl: −11.21 to | | 95% Cl: −20.50 to | | 95% Cl: −23.64 to | |
| Pneumococcus | 21.92 (ns) | | 22.63 (ns) | | 17.54 (ns) | | 21.70 (ns) | |
| (mg/L) | | | | | | | | |
| <35 | 39.0% | 43.2% | 38.3% | 40.8% | 37.9% | 43.1% | 45.6% | 43.6% |
| <50 | 53.7% | 55.9% | 52.1% | 54.1% | 50.6% | 60.3% | 54.4% | 63.6% |
| IgG-Tetanus | Non-Gaussian | | Non-Gaussian | | Non-Gaussian | | Non-Gaussian | |
| (IU/ml) | distribution (ns) | | distribution (ns) | | distribution (ns) | | distribution (ns) | |
| <0.1 | 12.2% | 13.5% | 10.6% | 13.3% | 11.5% | 13.8% | 12.3% | 14.6% |
| IgG- | Non-Gaussian | | Non-Gaussian | | Non-Gaussian | | Non-Gaussian | |
| Haemophilus | distribution (ns) | | distribution (ns) | | distribution (ns) | | distribution (ns) | |
| B (mcg/ml) | | | | | | | | |
| <1.0 | 51.2% | 55.9% | 51.1% | 54.1% | 49.4% | 53.5% | 50.9% | 60.0% |
| Sample size: | 118 | 85 | 89 | 77 | 84 | 45 | 54 | 45 |
| IgA- | 31 ± 3.97*** | 58.4 ± | 30.8 ± | 58.8 ± | 31.6 ± | 49.9 ± | 30.7 ± | 61.3 ± |
| Pneumococcus | | 6.7 | 4.7†††† | 7.0 | 4.9††† | 7.6 | 5.7†††† | 9.5 |
| (U/) | | | | | | | | |
| IgM- | 58.5 ± | 85 ± | 59.8 ± | 85.8 ± | 60.4 ± | 78.6 ± | 61.6 ± | 91.7 ± |
| Pneumococcus | 4.2*** | 6.9 | 4.9** | 7.4 | 5.1* | 7.1 | 7.0†† | 10.3 |
| (U/L) | | | | | | | | |

Data shown as mean ± 1 SEM unless otherwise stated.
*Independent T test (normally distributed) or †Mann-Whitney (non-normally distributed)
Levels of significance: */†p < 0.05, **/††p < 0.005, ***/†††p < 0.0005, ****/††††p < 0.0001

The association of clozapine with reduced IgG, IgA, IgM and Pneumococcal IgA and IgM remained statistically significant in all subgroups with 95% confidence intervals including when psychiatric diagnoses were restricted to schizophrenia only (Column C), and when non-smokers were excluded (Column D). When secondary causes of antibody deficiency were excluded (Column B) the odds ratios (with 95% confidence interval) for reduced immunoglobulins were IgG 9.02 (1.11-73.7), IgA: 32.6 (1.91-558) and IgM: 2.86 (1.42-5.73). In addition, a longer duration of clozapine therapy is associated with lower serum IgG levels (p 0.014) shown in FIG. 2. This is not observed in clozapine-naïve patients treated with alternative antipsychotic drugs, despite a longer treatment duration than the clozapine therapy group.

### Immunological Assessment of Referred Patients Taking Clozapine

Thirteen patients on clozapine were independently referred for assessment of antibody deficiency to Immunology clinic. Two had previously been recruited to the study and the eleven others are not included in the study to avoid bias. Five of the thirteen patients had been identified through the all Wales calculated globulin screening program. It was thus possible to undertake a more detailed immunological assessment in this group of thirteen ‘real life’ patients to provide additional background information (Table 3).

TABLE 3
Immunological characteristics of the 13 referred clozapine patients

| Referral | | | Relevant | Clozapine | CSMB | | | Follow-up/ |
|---|---|---|---|---|---|---|---|---|
| Reason | Age | Smoking | Medication | duration | (6.5-29.1%) | | Intervention | months |
| Recurrent | 47 | 20 | Clozapine | >4 | IgG | 0.3% | Prophylactic antibiotics | 120 |
| respiratory | | pack years | 250 mg | | <1.34 | | Failure to respond to | |
| tract | | | Sodium | | IgA | | haemophilus and | |
| infection | | | Valproate | | <0.22 | | pneumococcal | |
| (12 per | | | 1 g | | IgM | | vaccination. | |
| year). | | | Risperidone | | <0.17 | | Commenced SCIg 9.6 g | |
| | | | | | | | weekly in nursing home. | |
| | | | | | | | Recently discontinued | |
| | | | | | | | clozapine due to | |
| | | | | | | | neutropenia. | |
| Low | 46 | 42 | Clozapine | 15 | IgG 5.24 | 2.77% | Prompt antibiotic | 69 |
| calculated | | pack | 575 mg | | IgA 0.49 | | therapy | |
| globulin | | years | Senna, | | IgM | | Durable pneumococcal | |
| Included in | | | fibrogel, | | 0.41 | | vaccine response | |
| study | | | cyclizine | | | | Continues clozapine | |
| Low | 51 | 34 | Clozapine | 5 | IgG 2.68 | 5.50% | Prophylactic antibiotics | 48 |
| calculated | | pack | 200 mg | | IgA 0.38 | | Failure to responds to | |
| globulin. | | years | Amisulpride | | IgM | | haemophilus and | |
| | | | | | <0.17 | | pneumococcal | |
| | | | | | | | vaccination. | |
| | | | | | | | Continues clozapine, | |
| | | | | | | | Considering | |
| | | | | | | | immunoglobulin | |
| | | | | | | | replacement | |
| Persistent | 63 | 60 | Clozapine | 7.5 | IgG 2.98 | 0.5% | Prophylactic antibiotics | 42 |
| cough for | | pack | 400 mg | | IgA | | Non-durable | |
| over a year | | years | Olanzapine | | <0.22 | | pneumococcal vaccine | |
| and | | | Trihexyphenidyl | | IgM | | response | |
| remains | | | | | 0.23 | | Commenced IVIg 40 g 3 | |
| productive | | | | | | | weekly | |
| of green | | | | | | | Clozapine stopped with | |
| sputum | | | | | | | resultant psychotic | |
| despite | | | | | | | episode. | |
| several | | | | | | | Clozapine restarted | |
| courses of | | | | | | | with GCSF cover | |
| antibiotics. | | | | | | | Continues on SCIg and | |
| | | | | | | | clozapine. | |
| Recurrent | 49 | 55 | Clozapine | 7 | IgG 1.2 | 0.14% | Prophylactic antibiotics | 32 |
| respiratory | | pack | 300 mg | | IgA | | Failure to respond to | |
| infections | | years | Sodium | | undetect- | | pneumococcal | |
| Low | | | Valproate, | | able | | vaccination. | |
| calculated | | | Pirenzapine, | | IgM | | IVIg 40 g 3 weekly | |
| globulins | | | aripiprazole | | 0.07 | | Continues clozapine | |
| Recurrent | 63 | 20 | Clozapine | 10 | IgG 3.3 | 1.58% | Prophylactic | 24 |
| chest | | pack | 250 mg- | years | IgA 0.26 | | azithromycin: 4 chest | |
| infections | | years, | stopped | Stopped | IgM | | infections in 3 months | |
| Low | | stopped | Lithium | 24 | 0.41 | | Failure to respond to | |
| calculated | | 30 | 400 mg | months | | | pneumococcal | |
| globulin | | years | Levothyroxine | ago | | | vaccination | |
| | | ago | Calchichew | | | | Clozapine stopped-red | |
| | | | Citalopram | | | | flags with neutropenia | |
| | | | | | | | IgG rose to 5.95 from | |
| | | | | | | | 3.3 g/L, IgA 0.29, IgM | |
| | | | | | | | 0.49 after 24 months | |
| | | | | | | | CSMB rose to 2.77% | |
| 7 courses | 59 | 47 | Clozapine | 10 | IgG 2.38 | 2.54% | Prophylactic antibiotics | 15 |
| of | | pack | 450 mg | | IgA | | Failure to respond to | |
| antibiotics | | years | Omeprazole, | | <0.22 | | pneumococcal | |
| for chest | | | pirenzapine, | | IgM | | vaccination. | |
| infections | | | venlafaxine, | | <0.17 | | Commenced IVIg 30 g 3- | |
| past 12 | | | metformin, | | | | weekly | |
| months, 9 | | | saxagliptin, | | | | Continues clozapine | |
| GP visits | | | atorvastatin | | | | | |
| No | | | | | | | | |
| clozapine | | | | | | | | |
| red-flags | | | | | | | | |
| Included in | | | | | | | | |
| study | | | | | | | | |
| Recurrent | 46 | 74 | Clozapine | 21 | IgG 4.24 | 0.84% | Prophylactic antibiotics | 12 |
| respiratory | | pack | 450 mg | | IgA | | Failure to respond to | |
| infections | | years | Sertaline, | | <0.22 | | pneumococcal | |
| | | | montelukast, | | IgM | | vaccination. | |
| | | | simvastatin, | | <0.17 | | Commenced SCIg | |
| | | | seretide, | | | | Continues clozapine | |
| | | | salbulatamol, | | | | | |
| | | | temazepam | | | | | |
| Recurrent | 50 | 60 | Clozapine | >7 | IgG 6.65 | 4.95% | Prophylactic antibiotics | 12 |
| respiratory | | pack | 700 mg | | IgA | | Failure to responds to | |
| tract | | years | Amisulpride, | | <0.22 | | haemophilus and | |
| infections | | | cholecalciferol, | | IgM | | pneumococcal | |
| | | | cod | | <0.17 | | vaccination. | |
| | | | liver oil | | | | Continues clozapine | |
| Low | 51 | 12 | Clozapine | 11 | IgG 5.61 | 2.10% | Prompt antibiotic | 6 |
| calculated | | pack | 575 mg | | IgA 0.81 | | therapy | |
| globulin | | years | Fibrogel, | | IgM | | Failure to respond to | |
| | | | lactulose, | | 0.18 | | pneumococcal | |
| | | | cod liver | | | | vaccination. | |
| | | | oil, | | | | Continues clozapine | |
| | | | citalopram | | | | | |
| Recurrent | 61 | 15/day | Clozapine | >4 | IgG 4.79 | 1.49% | Prompt antibiotics | 6 |
| skin | | | 325 mg | | IgA 0.63 | | Assessment of vaccine | |
| infections | | | Sodium | | IgM | | responses ongoing | |
| | | | valproate, | | <0.17 | | Continues clozapine | |
| | | | metformin, | | | | | |
| | | | exenatide, | | | | | |
| | | | ciitalopram, | | | | | |
| | | | Fultium | | | | | |
| | | | D3, Omeprazole, | | | | | |
| Calculated | 36 | 35 | Clozapine - | Stopped | IgG 4.8 | N/A | Declined further blood | 5 |
| globulin | | pack | stopped 2 | | IgA 0.54 | | tests | |
| | | years | years prior | | IgM 0.3 | | | |
| | | | to referral | | | | | |
| | | | Procyclidine, | | | | | |
| | | | folic acid, | | | | | |
| | | | diazepam, | | | | | |
| | | | paracetamol | | | | | |
| Recurrent | 57 | 20-40 | Clozapine | >4 | IgG | 0.3- | Prophylactic antibiotics | 42 |
| respiratory | | pack | 750 mg | | <1.34 | 0.7% | Failure to respond to | |
| tract | | years | Amisulpride | | IgA | | pneumococcal | |
| infections. | | | | | <0.22 | | vaccination | |
| Clozapine- | | | | | IgM | | IVIg 40 g every 3 weekly | |
| induced | | | | | <0.17 | | Stopped clozapine | |
| sialorrhoea. | | | | | | | during chemotherapy | |

Certain additional analysis shown in FIGS. 1D, 3B, 4B and 5 was done on a slightly different set of referred clozapine patients comprising the 13 referred to in Table 3, plus 4 additionally recruited patients. In respect of FIG. 1D, 4 of the 17 patients were removed for various reasons therefore the number of patients for which data is presented is 13. In respect of FIG. 3B, the number of patients for which data is presented is shown in the Figure. In respect of FIG. 4B, the number of patients for which data is presented is stated below. In respect of FIG. 5, the number of patients for which data is presented is 15.

Immunoglobulins were reduced in all patients (mean IgG 3.6 g/L, IgA 0.34 g/L and IgM 0.21 g/L). There was no severe overall lymphopenia or B cell lymphopenia, however, all patients had a major reduction in the percentage of CSMB (mean 1.87%, reference range 6.5-29.1%). A substantial reduction of CSMB is characteristic of patients with common variable immunodeficiency (CVID), the commonest severe primary immunodeficiency in adults. The percentages of CSMB in these clozapine-treated and CVID patients compared to healthy controls are shown in FIG. 3A (p<0.0001). The plasmablast levels for 6 of the clozapine patients compared to CVID patients and healthy controls are shown in FIG. 4A (p=0.04) and in FIG. 3B with age matched CVID and healthy controls. A reduction of plasmablasts is also characteristic of patients with common variable immunodeficiency (CVID) and this was also observed in clozapine treated patients. Responses to vaccination were impaired in 10/11 patients assessed and management included emergency backup antibiotics for 2/13 patients, prophylactic antibiotics in 9/13 and 6/13 patients were treated with immunoglobulin replacement therapy (IGRT). No patients discontinued clozapine because of antibody deficiency. The inflammatory or granulomatous complications which occur in a subset of CVID patients were not observed.

Vaccine specific-IgG responses are routinely evaluated as part of clinical assessment and summarised in FIG. 4B. At initial assessment, levels below putative protective threshold were common with IgG to Haemophilus influenza B (HiB)<1 mcg/ml in 12/16 patients (75%); Pneumococcus-IgG <50 mg/L in 15/16 patients (94%); and Tetanus-IgG <0.1 IU/mL in 6/16 patients (38%) individuals tested. Post-Menitorix (HiB/MenC) vaccination serology was assessed after 4 weeks, with 5/12 (42%) individuals failing to mount a Haemophilus-IgG response ≥1 mcg/ml, and 1/12 failing to exceed the ≥0.1 IU/mL post-vaccination Tetanus-IgG level defined by the World Health Organisation. Following Pneumovax II, 8/11 (73%) individuals failed to develop an IgG response above a threshold of ≥50 mg/L.

FIG. 5 shows a gradual recovery in terms of the serum IgG level from 3.5 g/L to 5.95 g/L over 3 years but without clear improvement in IgA or IgM following cessation of clozapine.

One patient subsequently discontinued clozapine because of neutropenia which normalized on clozapine cessation. Over the following 24 months the serum IgG level gradually increased from 3.3 g/L to 4.8 g/L and then 5.95 g/L while IgA and IgM remained low. The increase in IgG was accompanied by a concomitant increase in class switched memory B cells from 1.58-2.77%, suggesting a gradual recovery on withdrawal of clozapine.

FIG. 1D shows a density plot showing distribution of serum immunoglobulin levels in patients receiving clozapine referred for Immunology assessment. Serum immunoglobulin distributions for clozapine-treated (n=94) and clozapine-naive (n=98) are also shown for comparison-adapted from (Ponsford et al., 2018a). Dotted lines represent the 5^{th} and 95^{th} percentiles for healthy adults. A leftward shift (reduction) in the distribution curves of total immunoglobulin is observed in patients on clozapine for each of IgG, IgA and IgM compared to clozapine naive patients; this finding was particularly marked for the additionally recruited clozapine referred patients.

### Summary of Results

Clozapine treatment in patients led to a significant reduction of all immunoglobulin types. Percentages of patients below the immunoglobulin reference ranges were higher in clozapine treated (n=123) as compared with clozapine naive patients (n=111) (IgG <6 g/L: 9.8% vs 1.8%; IgA <0.8 g/L: 13.1% vs 0.0%; IgM <0.5 g/L: 38.2% vs 14.2%) (p<0.0001) (see FIG. 1 A-C).

Extending the duration of clozapine treatment was associated with progressively reduced IgG levels in patients treated with clozapine but not in clozapine naive patients who were on other antipsychotic medication (see FIG. 2).

Notably the effect of clozapine on IgG levels was seen to be reversible, albeit slowly (years), consistent with an impact of clozapine on long-lived IgG+ plasma cells in particular.

Specific IgG antibodies were below protective levels in both clozapine-treated and clozapine-naïve groups (HiB 51.2% vs 55.9%; Pneumococcal 53.7% vs 55.9%; Tetanus 12.2% vs 13.5%)). However, pneumococcal IgA and IgM levels were significantly lower in clozapine-treated patients as compared with clozapine-naïve patients (IgA 31.0 U/L vs 58.4 U/L; IgM 58.5 U/L vs 85 U/L) (p<0.001) (see Table 2).

Mean levels of CSMBs were significantly reduced at 1.87% in clozapine-treated patients referred independently to clinic and not included in the overall study (n=12) and in CVID patients (n=54) as compared with healthy controls (n=36) and the reference range of 6.5-29.1% (p<0.0001) (see FIG. 3A). Mean levels of plasmablasts were also reduced in clozapine-treated patients (p=0.04).

FIG. 3B shows an extension of the data in FIG. 3A in which referred clozapine patients are compared to age matched CVID and health control subjects. The first graph shows that total B cell numbers are similar between clozapine, CVID and healthy controls and the second graph demonstrates no significant difference between clozapine treated and healthy control marginal zone B cell numbers while there is an increased number observed in CVID patients. The lower two graphs show a significant reduction in both CSMB and plasmablasts in both clozapine treated and CVID patients over healthy controls.

### Example 2

### Second Observational Study on Human Patients on Anti-Psychotic Therapy

Using a cross-sectional observational design in patients on anti-psychotic therapy, this study seeks to test the association between clozapine use, immunophenotype—specifically circulating B cell subsets and immunoglobulin levels—and documented infections, in comparison to other anti-psychotic medication. The study is recruiting patients established on clozapine and those on other antipsychotic drugs from Ashworth Hospital and outpatients from community mental health services in Mersey Care NHS Foundation Trust. The findings will partly provide validation of those from the initial observational study in an orthogonal population, in addition to extending insights into the impact of clozapine on B cell populations through more detailed immunophenotypic analysis.

The study entails a single blood test for detailed immunological analysis and completion of a clinical research form-based questionnaire detailing important clinical parameters including documented infection history, past medical history and concurrent medication use. The findings will be analysed to identify any association between clozapine, circulating B cell levels/function and immunoglobulin levels, its frequency and severity, as well as specificity in relation to other antipsychotic medications.

### Study Aims and Objectives

The specific research questions this study seeks to answer are:

### Primary Outcomes:

i) Is chronic treatment with clozapine associated with (a) a higher proportion of those with specific B cell subsets (namely class-switched memory B cells and plasma cells) below reference ranges and (b) a higher proportion of those with circulating immunoglobulin levels (IgG, IgA and IgM) below references compared to proportions below reference range observed in controls?

### Secondary Outcomes:

ii) Is clozapine associated with reductions in specific antibodies (e.g. pneumococcus, tetanus and Hib) compared to controls?
iii) Is clozapine use associated with an effect on circulating T cells (number/function) compared to controls?
iv) Is clozapine associated with a higher frequency of infections and antibiotic use than controls?
v) Are the primary outcomes related to duration of clozapine therapy?

### Immune Biomarkers

The following immune biomarkers are tested:
1. Total IgG IgM, IgA, and serum electrophoresis with immunofixation if appropriate;
2. Specific IgG levels—tetanus toxoid, pneumococcus, Hib (±IgA and IgM for pneumococcus);
3. Detailed immune cell phenotyping through FACS analysis, including:
   a. Lymphocyte phenotypes—(including CD3, CD4, CD8, CD19, CD56)
   b. B cell panel (based on the EUROCIass classification of B cell phenotype (Wehr et al., 2008)) which includes CSMB cells and plasmablasts
   c. Naïve T cell panel
4. RNA extraction from PBMCs (whole blood stored in a RNA preservation solution, e.g. Universal container with ^{˜}4-5 mL RNALater or in PAXgene tube to preserve RNA integrity) for subsequent RNA transcription analysis

All immune biomarker samples are processed and analysed in a UKAS Accredited validated NHS laboratory.

### Results

At the time of writing this study is still recruiting but an interim analysis of the available collected immunophenotypic data (approximately ⅔rds of the way through recruitment) has been undertaken with the caveat that this represent a proportion of the final projected sample size (n 100).

The major findings so far are detailed below:

a. Significantly reduced levels of circulating total IgG, IgA and IgM in patients on clozapine versus patients who have never taken clozapine (i.e. control, clozapine naive) (see FIG. 6A-C). These reductions are relatively greater for Ig of the A and M subclass. In addition, a trend to lower IgG antibodies against pneumococcus is present in those treated with clozapine (see FIG. 7).
b. Overall CD19⁺ B cell numbers are not significantly different between groups (see FIG. 8A-B).
c. Small increase in the number of naive (CD19⁺CD27⁻) B cells expressed as a proportion of total CD19⁺ B cells (see FIG. 9A-C).
d. Strong trends to a specific reduction in class-switched memory B cells (P=0.06 vs control, CD27⁺ IgM⁻ IgD⁻ as % B) in those treated with clozapine (see FIG. 11A-C) without perturbation of the overall memory B cell pool (see FIG. 10A-C) or IgM^{hi} IgD^{lo} memory B cell subpopulation (see FIG. 12A-C).
e. No significant difference between groups in circulating levels of transitional B cells or marginal zone B cells (See FIGS. 13A-C and 14A-C).
f. Strong trends to reduction in levels of plasmablasts in patients treated with clozapine (P=0.07 vs control clozapine naive) (see FIG. 15A-C).

### Example 3

### In Vivo Wild Type Mouse Study—Effect of Clozapine Versus Haloperidol

The impact of clozapine on B cell development, differentiation and function (inferred from circulating immunoglobulin levels) in primary (bone marrow) and secondary (spleen and also mesenteric lymph node) lymphoid tissue in wild type mice in the steady state (i.e. in the absence of specific immunological challenge) was assessed.

The specific objectives were to:

a) Determine the impact of clozapine on major B cell subsets in bone marrow and key secondary lymphoid organs (spleen and mesenteric lymph node) of healthy mice.
b) Define whether a dose-response relationship exists for clozapine on aspects of the B cell immunophenotype.
c) Assess the effect of clozapine administration on the circulating immunoglobulin profile of healthy mice.
d) Determine the specificity of clozapine's effect on the above readouts by comparison to another antipsychotic agent.

### Method

### Animals:

Young adult (age 7-8 weeks) C57BL/6 mature female mice were used for the study. Mice were housed at 22° C. in individually ventilated cages with free access to food and water and a 12-h light/dark cycle (8 a.m./8 p.m.). Mice acclimatised for 1 week on arrival prior to initiating experiments.

### Experimental Groups and Dose Selection:

Mice were allocated into one of five experimental groups as follows:

| | |
|---|---|
| 1. Control | saline |
| 2. Clozapine low dose | 2.5 mg/kg |
| 3. Clozapine intermediate dose | 5 mg/kg |
| 4. Clozapine high dose | 10 mg/kg |
| 5. Haloperidol | 1 mg/kg (intermediate dose) |

Dosing was given in staggered batches with each batch containing mice assigned to each experimental arm to reduce bias.

| | | Clozapine | Clozapine | Clozapine | Haloperidol | Mice |
|---|---|---|---|---|---|---|
| | | 2.5 | 5 | 10 | 1 | per |
| | Control | mg/kg | mg/kg | mg/kg | mg/kg | batch |
| Batch 1 | 2 | 2 | 2 | 2 | 2 | 10 |
| Batch 2 | 2 | 2 | 2 | 2 | 2 | 10 |
| Batch 3 | 2 | 2 | 2 | 2 | 2 | 10 |
| Batch 4 | 2 | 2 | 2 | 2 | 2 | 10 |
| Batch 5 | 2 | 2 | 2 | 2 | 2 | 10 |
| Batch 6 | 2 | 2 | 2 | 2 | 2 | 10 |
| Mice | 12 | 12 | 12 | 12 | 12 | 60 |
| per | | | | | | |
| group | | | | | | |

Dose selection was initially based on a literature review of studies administering these drugs chronically to mice (Ishisaka et al., 2015; Li et al., 2016; Mutlu et al., 2012; Sacchi et al., 2017; Simon et al., 2000; Tanyeri et al., 2017), the great majority of which had employed the intraperitoneal (IP) route of administration: clozapine (1.5, 5, 10, 25 mg/kg/day) (Gray et al., 2009; Moreno et al., 2013); haloperidol (0.25 mg/kg, 1 mg/kg/day) (Gray et al., 2009) and taking into account the LD50 for both drugs (clozapine 200 mg/kg, haloperidol 30 mg/kg).

Subsequently, pilot studies were undertaken to assess the impact of these, particularly of the higher doses of clozapine, to refine dose selection and maximise the welfare of treated mice. Clear dose-related sedative effects were evident from dosages of clozapine starting at 5 mg/kg, with marked psychomotor suppression (with respect to depth and duration) observed at the highest doses assessed (20 mg/kg and 25 mg/kg). In addition, effects on thermoregulation were also evident, necessitating use of a warming chamber and general supportive measures to defend thermal homeostasis. These adverse effects were consistent with the known (on-target) profile of clozapine in preclinical (Joshi et al., 2017; McOmish et al., 2012; Millan et al., 1995; Williams et al., 2012) and clinical settings (Marinkovic et al., 1994), with tolerance developing after the initial few days of dosing, as has been described in humans (Marinkovic et al., 1994).

Mice (n=12/group) were treated by once daily IP injection of the respective control solution/clozapine/haloperidol for 21 consecutive days.

### Biological Samples for Immunophenotyping:

At the end of the experimental period, mice were humanely euthanised and blood samples obtained for serum separation, storage at −80° C. and subsequent measurement of immunoglobulin profiles (including the major immunoglobulin subsets IgG1, IgG2a, IgG2b, IgG3, IgA, IgM, and both light chains kappa and lambda) by ELISA.

In parallel, tissue samples were rapidly collected from bone marrow (from femur), spleen and mesenteric lymph nodes for evaluation of cellular composition across these compartments using multi-laser flow cytometric detection and analysis.

### B Cell Immunophenotyping by Flow Cytometry:

Focused B cell FACS (fluorescence-activated cell sorter) panels were prepared separately for both primary (bone marrow) and secondary (spleen/lymph node) lymphoid tissue to allow an evaluation of drug impact on the relative composition of B cell subsets spanning the spectrum of antigen-independent and -dependent phases of B cell development.

Individual antibodies employed for flow cytometry panels were pilot tested in the relevant tissues (i.e. bone marrow, spleen and mesenteric lymph node) and the optimal dilution of each antibody determined to enable clear identification of subpopulations. FACS data were extracted by BD FACSymphony and analysed by FlowJo software.

### Results

### Body Weight:

Clozapine (CLZ) induced a transient fall in body weight at both 5 mg/kg and 10 mg/kg doses, maximal by 3 days but recovering fully to baseline by day 9 with progressive weight gain beyond this (see FIGS. 16 and 17). This finding is likely to reflect the sedative effect of clozapine on fluid/food intake during the initial few days of dosing, with evidence of tolerance to this emerging over the course of the experiment.

### Early B Cell Development in Bone Marrow:

B cells originate from hematopoietic stem cells (HSCs), multipotent cells with self-renewal ability, located in the bone marrow. This early B cell development occurs from committed common lymphoid progenitor cells and progresses through a set of stages, dependent on physical and soluble chemokine/cytokine interactions with bone marrow stromal cells, defined using cell surface markers.

The earliest B cell progenitor is the pre-pro-B cell, which expresses B220 and has germline Ig genes. Next, pro-B cells rearrange their H (heavy) chain Igμ genes, and express CD19 under the control of transcription factor Pax5. At the pre-B cell stage, cells downregulate CD43, express intracellular and then rearrange the L (light) chain and upregulate CD25 in an Irf4-dependent manner. Successfully selected cells become immature (surface IgM⁺IgD⁻) B cells. Immature B cells are tested for autoreactivity through a process of central tolerance and those without strong reactivity to self-antigens exit the bone marrow via sinusoids to continue their maturation in the spleen.

No overall reduction in B cells in the bone marrow (BM) was observed at any dose of clozapine (see FIG. 18). However, a significant increase in the proportion of very early B cell progenitors, the pre-pro B cells (i.e. B220⁺CD19⁻CD43⁺CD24^{lo}BP-1⁻IgM⁻IgD⁻) was observed with 10 mg/kg clozapine, without any change evident in the subsequent pro-B cell fraction (see FIG. 18). In contrast, no significant effect of haloperidol was evident on any of these early developing B cell subsets.

Examination of subsequent stages of B cell development in bone marrow revealed a reduction in pre-B cells (i.e. B220⁺CD19⁺CD43⁻CD24⁺BP−1⁻IgM⁻IgD⁻) in mice treated with clozapine (see FIG. 19). Notably this effect exhibited dose-dependency, with a significant difference observed verses control mice with even the lowest dose of clozapine employed (2.5 mg/kg). Furthermore, the percentage of pre-B cells that were proliferating (i.e. B220⁺CD19⁺CD43⁻CD24^{hi}BP−1⁺IgM⁻IgD⁻) was diminished with clozapine, reaching significance for the 5 mg/kg dose (see FIG. 19). Correspondingly, a reduction in the percentage of immature B cells in bone marrow was identified (i.e. B220⁺CD19⁺CD43⁻CD24⁺IgM⁺IgD⁻) (see FIG. 19).

Together, these findings suggest a specific impact of clozapine on early B cell development, with a modest arrest between the pre-pro-B cell and pre-B cell stages in the absence of specific immunological challenge.

### Peripheral B Cell Development—Total Splenic B Cells:

After emigrating from the bone marrow, functionally immature B cells undergo further development in secondary lymphoid organs, enabling further exposure to (peripheral) self-antigen and peripheral tolerance (resulting in cell deletion through apoptosis, anergy or survival). The majority of immature B cells exiting bone marrow do not survive to become fully mature B cells, a process regulated by maturation and survival signals received in lymphoid follicles, including BAFF (B cell activating factor) secreted by follicular dendritic cells.

Mice treated with clozapine at 5 mg/kg and 10 mg/kg were seen to have a significantly lower percentage of splenic B cells (i.e. B220⁺TCR−β⁻) expressed as a proportion of total live splenocytes (see FIG. 21). No effect was identified on other cell populations (i.e. B220⁻TCR−β⁻), which may include γδ T cells (which do not express the αβ T cell receptor, TCR), natural killer (NK) cells, or other rare lymphoid cell populations (see FIG. 21). This was accompanied by a reciprocal increase in the percentage of splenic T cells (i.e. B220−TCR−β+) (see FIG. 21). In contrast, activated T cells (i.e. B220+TCR−β+), reflecting a small proportion of total live splenocytes were reduced in dose-dependent fashion by clozapine compared to control, an effect also modestly apparent for haloperidol (see FIG. 21).

These findings suggest that clozapine, but not haloperidol, is able to affect peripheral (splenic) B cells in addition to the observed changes in bone marrow B cell precursors.

### Splenic B Cell Subpopulations:

Immature B cells exiting the bone marrow and entering the circulation are known as transitional B cells. These immature cells enter the spleen and competitively access splenic follicles to differentiate via transitional stages to immunocompetent naive mature B cells. This occurs sequentially in the follicle from transitional type 1 (T1) cells, similar to immature B cells in bone marrow, to type 2 (T2) precursors. The latter are thought to be the immediate precursor of mature naive B cells. T2 B cells have been demonstrated to show greater potency in response to B cell receptor stimulation than T1 B cells, suggesting that the T2 subset may preferentially undergo positive selection and progression into the long-lived mature B cell pool (Petro et al., 2002).

Transitional cells can differentiate into follicular B cells, representing the majority of peripheral B cells residing in secondary lymphoid organs, or a less numerous population, marginal zone (MZ) B cells residing at the white/red pulp interface which are able to respond rapidly to blood-borne antigens/pathogens.

Mice treated with clozapine were found to have a mildly reduced proportion of newly emigrated transitional stage 1 (T1) B cells in the spleen, including at the 2.5 mg/kg dose, which may in part reflect the reduction in percentage of bone marrow immature B cells (see FIG. 22). In contrast, a small increase in the proportion of T2 B cells was identified across all doses of clozapine (see FIG. 22), consistent with enhanced positive selection of T1 B cell subsets for potential progression into the long-lived mature B cell pool.

While clozapine administration reduced the splenic B cell contribution to live splenocytes (see FIG. 21), no specific reductions were identified in either splenic follicular (i.e. B220⁺CD19⁺CD21^{mid}CD23⁺) or marginal zone (i.e. B220⁺CD19⁺CD21⁺CD23^{Lo/-}) B cell subsets (see FIG. 22), suggesting that in the immunologically unchallenged state, clozapine administration in mice results in a global reduction in splenic B cell populations.

Germinal centres (GCs) are micro-anatomical structures which form over several days in B cell follicles of secondary lymphoid tissues in response to T cell-dependent antigenic (e.g. due to infection or immunisation) challenge (Meyer-Hermann et al., 2012). Within GCs, B cells undergo somatic hypermutation of their antibody variable regions, with subsequent testing of the mutated B cell receptors against antigens displayed by GC resident follicular dendritic cells. Through a process of antibody affinity maturation, mutated B cells which higher affinity to antigen are identified and expanded. In addition, class switch recombination of the immunoglobulin heavy chain locus of mature naive (IgM⁺IgD⁺) B cells occurs before and during GC reactions, modifying antibody effector function but not its specificity or affinity for antigen. This results in isotype switching from IgM to other immunoglobulin classes (IgG, IgA or IgE) in response to antigen stimulation.

GCs are therefore sites of intense B cell proliferation and cell death, with outcomes including apoptosis, positive selection for a further round of somatic hypermutation (i.e. cyclic re-entry), or B cell differentiation into antibody secreting plasma cells and memory B cells (Suan et al., 2017). In the steady state, GC cells (i.e. B220+CD19+IgD−CD95+GL−7+) formed a very small proportion of total live B cells in the spleen, with no differences observed versus control or haloperidol in response to clozapine administration (see FIG. 22).

Bone Marrow Antibody Secreting Cell Populations:

Antibody secreting cells represent the end-stage differentiation of the B cell lineage and are widely distributed in health across primary and secondary lymphoid organs, the gastrointestinal tract and mucosa (Tellier and Nutt, 2018). These cells all derive from activated B cells (follicular, MZ or B1). Plasmablasts, representing short-lived cycling cells, can be derived from extra-follicular differentiation pathway in a primary response (producing relatively lower affinity antibody), as well as from memory B cells that have undergone affinity maturation in the GC (Tellier and Nutt, 2018).

Plasmablasts developing in GCs can leave the secondary lymphoid organ and home to the bone marrow. Here, only a small proportion are thought to be retained and establish themselves in dedicated micro-environmental survival niches to mature into long-lived plasma cells (Chu and Berek, 2013), a process thought to be regulated by docking onto mesenchymal reticular stromal cells (Zehentmeier et al., 2014) and requiring haematopoietic cells (e.g. eosinophils) (Chu et al., 2011), the presence of B cell survival factors (e.g. APRIL and IL-6) (Belnoue et al., 2008) and hypoxic conditions (Nguyen et al., 2018).

In the healthy state, the bone marrow houses the majority of long-lived plasma cells. Clozapine at 5 and 10 mg/kg induced a significant reduction in the percentage of long-lived plasma cells in the bone marrow (i.e. B220^{lo}CD19⁻IgD⁻IgM⁻CD20⁻CD38⁺⁺CD138⁺) by ^{˜}30% compared to control (see FIG. 20). In contrast, no effect of haloperidol was seen on this specific B cell population (see FIG. 20). No significant changes were detected in either class-switched memory B cells (i.e. B220⁺CD19⁺CD27⁺IgD⁻IgM⁻CD20⁺CD38^{+/-}) or plasmablasts (i.e. B220^{lo}CD19⁺CD27⁺IgD⁻IgM⁻CD20⁻CD38⁺⁺) in the bone marrow with any treatment, however both these represent a very small proportion of total B cells in the bone marrow in the immunologically unchallenged steady state (see FIG. 20).

These findings indicate that clozapine can exert a specific effect to reduce the proportion of long-lived plasma cells in the bone marrow, a population thought to be the major source of stable antigen-specific antibody titres in plasma involved in humoral immune protection and, in pathogenic states, stable autoantibody production.

### Circulating Immunoglobulin Levels:

Clozapine administration at both 5 and 10 mg/kg resulted in a reduction in circulating IgA levels compared to control, an effect not observed with haloperidol (see FIG. 24; P, positive control; N, negative control). No other isotype classes were affected under the experimental conditions used (see FIG. 24).

### Mesenteric Lymph Nodes:

Under the current experimental conditions, no significant differences were identified between any of the groups in lymphocyte subpopulations assessed in mesenteric lymph nodes (MLN) (see FIG. 23).

### Conclusion

This study investigated the potential for clozapine to influence the immunophenotype of wild type mice in the steady state, specifically B cell subpopulations, with functional impact inferred through circulating levels of immunoglobulins. The major findings of this study are that 3 weeks parenteral (I.P.) administration of clozapine:

a) Increases the proportion of pre-pro-B cells while reducing the proportion of later-stage pre-B cells and immature B cells in the bone marrow.
b) Reduces the proportion of live splenocytes that are B cells.
c) Exerts subtle effects on developing B cells in the spleen, specifically transitional B cell populations in favouring a greater proportion of T2 type cells.
d) Significantly reduces the proportion of long-lived plasma cells in the bone marrow.
e) Impacts on circulating immunoglobulin levels, specifically lowering IgA.
f) Results in a dose-dependent decrease in the proportion of activated T cells in spleen which, in contrast to all the above findings, was also observed with the dose of haloperidol used.

Taken together, these observations indicate that clozapine exerts complex effects on B cell maturation in vivo, not limited to the late stages of B cell differentiation or activation. Specifically, the findings suggest that clozapine can influence the maturation of early B cell precursors, with a partial arrest of antigen-independent B cell development in the bone marrow.

In parallel, clear effects of clozapine are identified on peripheral B cell subpopulations, with a notable impact on reducing the overall B cell proportion of live splenocytes, and on long-lived antibody secreting plasma cells in the bone marrow. An impact on antibody secreting cells is likely to underlie the observed significant reduction in circulating IgA, particularly striking given the otherwise immunologically unchallenged state of the mice.

Notably, the impact on B cell subpopulations was not observed with a comparator antipsychotic agent, haloperidol, consistent with specificity of action of clozapine on B cell maturation. While the current experiments do not enable a distinction between a direct or indirect effect of clozapine on bone marrow, peripheral and late B cell populations, taken together with findings from separate in vitro B cell proliferation assays, an indirect effect is deemed more likely. This may involve a variety of other myeloid, lymphoid (e.g. T follicular helper cells) and/or (mesenchymal) stromal supportive cells.

### Example 4

### Mouse Collagen-Induced Arthritis (CIA) Model Study—Effect of Clozapine

The CIA model is a well-established experimental model of autoimmune disease. The inventors have employed the CIA model as a highly clinically relevant experimental system in which B cell-derived pathogenic immunoglobulin made in response to a sample specific antigen drives autoimmune pathology to explore the potential efficacy of clozapine and its associated cellular mechanisms.

### Method

### Animals:

Adult (age 13-15 weeks) DBA/1 male mice were purchased from Envigo (Horst, Netherlands). Mice were housed at a 21° C.±2° C. in individually ventilated cages with free access to food and water and a 12-h light/dark cycle (7 am/7 pm). Mice were acclimatised for 1 week on arrival prior to initiating experiments.

### Experimental Groups and Dose Selection:

Mice were allocated into one of five experimental groups as follows:

1. Control saline
2. Clozapine 5 mg/kg treatment from day 15 after immunization
3. Clozapine 10 mg/kg treatment from day 15 after immunization
4. Clozapine 5 mg/kg treatment from day 1 after immunization
5. Clozapine 10 mg/kg treatment from day 1 after immunization

Mice (n=10/group) were treated by once daily IP injection of the respective control solution/clozapine until day 10 after onset of clinical features of arthritis. All experiments were approved by the Clinical Medicine Animal Welfare and Ethical Review Body (AWERB) and by the UK Home Office.

### Anti-Arthritic Effect of Clozapine In Vivo:

DBA/1 mice were immunised with bovine type II collagen in CFA and monitored daily for onset of arthritis. Clozapine was administered daily by intraperitoneal injection at doses of 5 mg/kg or 10 mg/kg. Controls received vehicle (saline) alone. Treatment of mice commenced in one experiment on day 1 after immunisation and in a second experiment on day 15 after immunisation. Clinical scores and paw-swelling were monitored for 10 days following onset of arthritis. A clinical scoring system was used as follows. Arthritis severity was scored by an experienced, non-blinded investigator as follows: 0=normal, 1=slight swelling and/or erythema, 2=pronounced swelling, 3=ankylosis. All four limbs were scored, giving a maximum possible score of 12 per animal.

At the end of the experimental period, mice were humanely euthanised and bled by cardiac puncture to obtain blood samples for serum separation, storage at −80° C. and subsequent measurement of specific anti-collagen immunoglobulin (IgG1 and IgG2a isotypes) by ELISA. In parallel, spleen and inguinal lymph nodes were harvested for evaluation of cellular composition across these compartments using multi-laser flow cytometric detection and analysis. Numbers of B cell subsets in spleen and lymph nodes were determined by FACS.

### Statistical Analysis:

Data were analyzed by one-way ANOVA with Tukey's or Dunnett's multiple comparison test or two-way ANOVA with Tukey's multiple comparison test as appropriate. All calculations were made using GraphPad Prism software. A P value less than 0.05 was considered significant.

### Results

### Effect of Clozapine on Onset, Clinical Score and Paw-Swelling:

Treatment of mice with clozapine was significantly effective in delaying the onset of arthritis post-immunisation (see FIGS. 25 and 26). In particular, treatment with both doses of clozapine from day 1 was extremely effective in delaying arthritis onset (see FIGS. 25 and 26).

Furthermore, treatment with both doses of clozapine reduced overall clinical score when administered on day 1 and, in the case of 10 mg/kg clozapine, also reduced swelling of the first affected paw (see FIG. 27). Clozapine administration also reduced the total number of affected paws compared to vehicle control, an effect significant with dosing at D1 (see FIG. 28).

### Effect of Clozapine on Peripheral B Cell Subsets:

Mice treated with clozapine at all doses and time points (i.e. 5 mg/kg or 10 mg/kg from day 1 or day 15) were seen to have a significantly lower percentage of B220⁺ B cells in lymph nodes (see FIG. 29). In addition, clozapine administered at 10 mg/kg from day 1 also significantly reduced the proportion of B220⁺ B cells in spleen.

Under the experimental conditions employed, no significant effect of clozapine was observed on plasma cell numbers in lymph node, however a significant reduction in the proportion of plasma cells was identified in spleen at a dose of 10 mg/kg clozapine given on day 1, with nominally lower values for plasma cells as a proportion of live cells at every other dose/time evaluated compared to control (see FIG. 30).

Strikingly significant reductions in lymph node follicular B cells (B220⁺IgD⁻Fas⁺GL7^{hi}) were observed in mice treated with clozapine across all doses/both time points (see FIG. 31). In addition, the level of GL7 expression on follicular B cells in lymph node were significantly decreased across all clozapine treatment groups compared to vehicle treated controls (see FIG. 32). There was evidence of dose- and time-dependency of effect with particularly profound reductions in GL7 expression in mice treated with clozapine from day 1 (see FIG. 32).

### Effect of Clozapine on Anti-Type II Collagen IgG Isotypes:

Clozapine administration at 5 or 10 mg/kg from day 1 or day 15 had no significant impact on serum IgG2a measured at a single time point. However, clozapine administration led to nominal reductions in levels of IgG1 across all doses tested, reaching statistical significance for the group treated with 10 mg/kg from day 15 (see FIG. 33).

### Effect of Clozapine on T Follicular Helper Cells:

Treatment of mice with 5 mg/kg or 10 mg/kg of clozapine from day 1 or day 15 did not significantly affect proportions of CD4⁺PD1⁺CXCR5⁺ T follicular helper cells in lymph node or spleen (see FIG. 34). However, analysis of mean fluorescence intensity (MFI) revealed robust reductions in expression of PD-1 and CXCR5 on T follicular helper cells in mice-treated with clozapine (see FIGS. 35 and 36). Reduced expression of PD-1 in lymph node T follicular helper cells was evident for clozapine at all doses and time points evaluated (see FIG. 35). In the case of CXCR5 expression, significant reductions were observed in mice dosed with clozapine from day 1 and evident in both lymph node (strongest signal for reduction) and spleen (see FIG. 36). In addition, a reduction in expression of CCR7 was observed on germinal centre resident T follicular helper cells in both lymph node and spleen of mice treated with clozapine (see FIG. 37).

### Conclusion

This study investigated the potential for clozapine to ameliorate CIA and its impact on major B cell subsets. The major findings of this study are as follows.

a) Clozapine is extremely effective at delaying disease onset in the CIA model.
b) Clozapine ameliorates the severity in CIA.
c) Clozapine reduces the proportion of B220⁺ B cells in both spleen and lymph node.
d) Clozapine reduces the proportion of splenic plasma cells.
e) Clozapine results in substantial reduction in the proportion of lymph node follicular B cells (IgD⁻Fas⁺GL7^{hi}) in B220⁺ B cells and lowers their expression of GL-7.
f) Clozapine demonstrated some ability to reduce pathogenic immunoglobulin, specifically anti-collagen IgG1 (at a dose of 10 mg/kg dosed from D15 after immunisation) in the context of the experimental conditions assessed (single time point immunoglobulin measurement).
g) Clozapine markedly reduces the expression of PD1 and CXCR5, in addition to CCR7, on lymph node T follicular helper cells (PD1⁺CXCR5⁺) without impacting upon the proportion of cells.

Taken together, these observations indicate that clozapine delayed disease onset, probably through multiple mechanisms likely to involve its impact on (secondary) lymphoid tissue and its ability to form functional germinal centres with subsequent impact on antibody producing B cells.

Specifically, clozapine is seen to reduce germinal centre B cells in local lymph node [marked by expression of GL7 in immunised spleen/lymph node (Naito et al., 2007)] following immunisation. GL7^{hi} B cells exhibit higher specific and total immunoglobulin production in addition to higher antigen-presenting capacity (Cervenak et al., 2001). Thus the observation of a reduction in surface expression of the GL7 epitope with clozapine suggests an impact to lower functional activity of these B cells for producing antibody and presenting antigen.

In parallel, clozapine is seen to affect T follicular helper cells, a critical T cell subset which controls the formation of and coordinates the cellular reactions occurring within germinal centres that is essential for somatic hypermutation, isotype class switching and antibody affinity maturation, differentiating B cells into memory B cells or plasma cells. T follicular helper cells therefore specialise in promoting the T cell-dependent B cell response (Shi et al., 2018). In particular, while not affecting the overall proportion of T follicular helper cells, clozapine is seen to reduce PD1 (programmed cell death-1) expression which is essential for proper positioning of T follicular helper cells through promoting their concentration into the germinal centre from the follicle (Shi et al., 2018). PD1 is also required for optimal production of IL-21 by T follicular helper cells, with PD1-PD-L1 interactions (i.e. the cognate ligand of PD1) between T follicular helper cells and germinal centre B cells aiding the stringency of affinity-based selection.

Furthermore, clozapine was seen to reduce the expression of CXCR5 on T follicular helper cells. CXCR5 (CXC chemokine receptor 5) is regarded as the defining marker for these cells; upregulation of CXCR5 enables relocation to the T/B border and, through attraction to CXCL-13, the B cell zone of lymphoid tissue to allow T follicular helper cells to enter the B cell follicle (Chen et al., 2015). Accordingly, reduced expression of CXCR5 on T follicular helper cells would impede their migration into B cell follicles and thereby reduce their ability to localise and interact with germinal centre B cells. Consistent with this, mice deficient in CXCR5 or selectively lacking CXCR5 on T cells display complete resistance to induction in CIA, in concert with reduced secondary lymphoid germinal centre formation and lower anti-collagen antibody production (Moschovakis et al., 2017).

Clozapine was also found to reduce expression of CCR7 on T follicular helper cells. CCR7 downregulation is regarded as an important mechanism through which activated CD4⁺ T cells overcome T zone chemokines which promote retention in the T zone (Haynes et al., 2007). Importantly, promotion of normal germinal centre responses by T follicular helper cells requires a coordinate upregulation of CXCR5 and downregulation of CCR7 (Haynes et al., 2007). Thus, the balanced expression of CXCR5 and CCR7 is critical to fine tuning of T follicular helper cell positioning and efficient provision of B cell help (Hardtke et al., 2005). The observation that clozapine can influence both CXCR5 and CCR7 expression on T follicular helper cells is therefore consistent with an ability of clozapine to perturb positioning and proper function of these cells, vital for T cell support of production of high affinity antibodies in response to T dependent antigens.

Further highlighting the importance of germinal centre formation to the pathogenesis of CIA is the finding that syndecan-4 null mice, which exhibit lower numbers of B cells and deficient germinal centre formation in draining lymph nodes, are resistant to CIA (Endo et al., 2015). Given the critical importance of tight regulation of germinal centres to the maintenance of self-tolerance and prevention of pathogenic autoantibody production in autoimmunity, the impact of clozapine as demonstrated in the CIA model strongly supports its potential to mitigate pathogenic autoantibody production.

### Example 5

### Study of Effect of Clozapine and Norclozapine on Human Plasma Cell Generation Using an In Vitro B Cell Differentiation System

An established in vitro platform (Cocco et al., 2012) was used to evaluate the impact of clozapine, its major metabolite norclozapine and a comparator antipsychotic drug, haloperidol, on the generation and differentiation and viability of human plasma cells.

### Method

### General:

The system employed is based on a published model (Cocco et al., 2012) which uses a CD40L/IL-2/IL-21 based stimulus to drive B-cell activation and differentiation in a 3-step process to generate plasmablasts and functional polyclonal mature plasma cells (See FIG. 38). The final step of the culture (Day 6-9) was performed in the context of IFN-α driven survival signals and without stromal cells.

The experiment was performed using total peripheral blood B-cells isolated from healthy donors. The experiment was performed from four independent donors.

### Drug Addition:

Compounds were sourced from Tocris and dissolved in DMSO at the following concentrations:

### Clozapine:

350 ng/ml Clozapine (approximately equivalent to 500 mg adult human dose)
100 ng/ml Clozapine
25 ng/ml Clozapine (approximately equivalent to 55 mg adult human dose)

### Norclozapine:

200 ng/ml norclozapine
70 ng/ml norclozapine
15 ng/ml norclozapine

### Haloperidol:

25 ng/ml Haloperidol
8 ng/ml Haloperidol
2 ng/ml Haloperidol

DMSO as diluent control at 0.1%. All DMSO concentrations were adjusted to 0.1% for all drug treated samples.

Drugs were added at two time points:
day-3 of the culture (activated B-cell/pre-plasmablast), or
day-6 of the culture (plasmablast)

### Evaluation:

The cultures were evaluated 3 days after addition of the compound with day-3 drug additions evaluated at day-6 (plasmablast) and day-6 drug additions evaluated at day-9 (early plasma cell) (see FIG. 38).

### Evaluation Encompassed:

### Flow Cytometric Assessment of:

phenotype (CD19, CD20, CD27, CD38, CD138)
viability (7AAD)
cell number (bead count)

### Immunoglobulin Secretion:

ELISA analysis of total IgM/IgG from bulk supernatant collected at day 6 and day 9 of respective cultures

### Results

### Cell Phenotype:

Across all four donors the control DMSO samples demonstrated a transition to a plasmablast state from day 3 to day 6 with downregulation of CD20, upregulation of CD38 and variable upregulation of CD27 combined with retained CD19 expression and lack of CD138. On subsequent transfer into plasma cell maturation conditions the control cells showed progressive loss of CD20, downregulation of CD19 and upregulation of CD138 combined with further upregulation of CD38 and CD27 indicating transition to early plasma cell state. These findings indicate that the differentiation protocol worked in relation to phenotype and that all four samples were suitable as references for the in vitro differentiation system.

In terms of effects on phenotypic maturation none of the drugs at any concentration showed significant effects on the downregulation of the B cell phenotype as reflected in equivalent loss of CD20 and CD19 expression. None of the drugs at any concentration showed significant effects on the pattern of acquisition of C27 or CD138 expression at either day 6 or day 9 time points.

All three drugs showed a dose related effect on the expression of CD38 in one donor. This was modest at the day 6 time point but was significant at the day 9 time point with a substantial and reproducible shift in CD38 expression. However, this effect was not observed as a consistent effect across the other donors.

### Cell Number and Viability:

Across all four donors the control DMSO samples demonstrated an expansion to the plasmablast state from day 3 to day 6 and contraction during the transition to plasma cell state. Based on an input activated B cell number at day 3 of 10⁵ the average expansion observed during the day 3 to day 6 culture was 12-fold. There was a 5-fold contraction that accompanied the maturation to the plasma cell state from 5×10⁵ input at day 6 to 10⁵ viable cells at day 9. It was concluded that the differentiation protocol worked in relation to cell number and that all four samples are suitable as references.

None of the drugs at any concentration impacted significantly on the number of viable cells at either day 6 or day 9. This was not affected whether considering total cell number or viable cell number per input cell. Based on equivalent input activated B cell number the degree of expansion from day 3 to day 6 was equivalent across all drugs and concentrations. Equally there was no effect on the viable cell number recovered at day 9 with any drug at any concentration.

### Immunoglobulin Secretion:

Across all four donors the control DMSO samples showed evidence of significant IgM and IgG secretion at across the day 3 to day 6 culture. This was continued into the day 6 to day 9 culture with predicted higher per cell estimated secretion rates in this second culture phase to the plasma cell stated. It was concluded that the differentiation protocol worked in relation to immunoglobulin secretion and that all four samples are suitable as references.

In terms of immunoglobulin secretion there is greater variation between individual donors, but there were no clear trends in response to any of the three drugs at any dose. Normalising to DMSO as control provided the simplest view of the data and showed only minor shifts in the detected immunoglobulin in relation to IgG. Where changes are observed these follow inverse responses in relation to the dose for example norclozapine with one donor.

### Conclusion

The results showed that none of the drugs are directly toxic to differentiating B-cells, nor do any of the drugs at any concentration show consistent effects on the ability of the resulting differentiated antibody secreting cells to secrete antibody.

In terms of phenotypic responses there is variability between the donors in relation to CD38 expression with one donor in particular showing an apparent dose dependent downmodulation in the window of differentiation between plasmablast (day 6) and early plasma cell (day 9). However this response did not reproduce as a consistent feature across the other donors tested.

Overall, therefore, the compounds as tested do not show a consistent inhibitory effect on the functional or phenotypic maturation of activated B-cells to the early plasma cell state and have no effect on viability of antibody secreting cells.

The in vitro system employed has limitations in terms of being a ‘forced’ B cell differentiation assay (as opposed to physiological expansion), with a focus on peripheral B cells, limited culture duration which may not reflect effects of very chronic exposure, and lack of the normal micro-environment of B cells in primary (e.g. bone marrow) or secondary lymphoid tissues, nor indirect regulation (e.g. through T follicular helper cells and/or IL-21). Notwithstanding these, the findings suggest that clozapine is unlikely to be acting directly on plasma cells or their precursors and that the immunophenotypic findings in vivo reflect a more complex and/or indirect action. The findings from this in vitro study are consistent with the lack of reduction in overall B cell numbers (i.e. no evidence of generalized B cell depletion in patients taking clozapine).

### Summary of Results Set Out in Examples 1-5:

The results set out in the examples above, encompassing observational data in humans treated with clozapine for prolonged periods of time, to short term dosing in healthy wild type mice in an immunologically unchallenged setting, to evaluation in a disease model of autoimmune disease with a major B cell component driven by antigen (CIA model), highlight several key effects of clozapine:

1. Reduction in total circulating immunoglobulin levels affecting all classes evaluated (IgG, IgM and IgA). While exhibiting interindividual variation, clozapine is seen to result in a leftward shift in the frequency distribution curve for these immunoglobulins. The robustness of this finding is highlighted by the interim findings in an orthogonal cohort of patients taking clozapine or other antipsychotics.

2. A relatively greater impact in human to reduce IgA (and IgM) compared to IgG, in part recapitulated with short-term dosing of wild type mice.

3. Evidence of progressive immunoglobulin (IgG) reduction with increasing duration of clozapine exposure in human. Conversely, evidence of gradual recovery (over years) of IgG on clozapine cessation.

4. Reduction in specific immunoglobulin. Beyond reductions in total immunoglobulin titre, clozapine is seen to lower pathogenic immunoglobulin (CIA model) and has been demonstrated by the inventors to lower pneumococcal specific antibody in human (Ponsford et al., 2018b), with the latter demonstrating a strong trend to significantly lower values on even interim analysis of the second observational cohort.

5. No significant impact on overall circulating (CD19+) B cells numbers. This observation contrasts sharply with the impact of current aggressive generalised B cell depleting biological approaches.

6. Substantial reductions in circulating plasmablasts (short-lived proliferating antibody secreting cells of the B cell lineage) and class-switched memory B cells. Both cell types are critical in the immediate and secondary humoral response. Class-switching enables a B cell to switch from IgM to production of the secondary IgH isotype antibodies IgG, IgA or IgE with different effector functions (Chaudhuri and Alt, 2004). Increased class-switching and plasma cell differentiation is recognised as a key feature in autoimmune disease associated with pathogenic immunoglobulin production (Suurmond et al., 2018). An ability of clozapine to inhibit this process, i.e. reduce class-switched memory B cells, suggests particular therapeutic potential in the setting of pathogenic immunoglobulin-mediated disorders which are primarily mediated by autoantibodies of the IgG, IgA or IgE subclass.

7. Subtle effects on bone marrow B cell precursors, specifically including a reduction in total pre B cells, proliferating pre B cells and immature B cells. This is notable for being a key endogenous transition checkpoint of B cell development for autoreactivity (Melchers, 2015). Defective B cell tolerance, including early tolerance, is recognised as a fundamental feature predisposing to autoimmunity (Samuels et al., 2005; Yurasov et al., 2005). Accordingly, while speculative, it is possible that this effect of clozapine will serve to reduce further progression of B cells with autoreactivity (of the IgH chain) to modulate the emerging B cell repertoire.

8. Reduction in bone marrow long-lived plasma cells, a key cell population responsible for driving persistent autoimmune disease through the production of pathogenic immunoglobulin and which is substantially refractory to existing therapeutics.

9. The ability to substantially delay the onset of an experimental model of autoimmune disease with a substantial B cell-driven and pathogenic autoantibody component.

10. Disruption of germinal centre function through effects on its key cellular components: induction of a profound reduction in germinal centre B cells together with reduction in surface expression of key proteins regulating T follicular helper cell functionality (PD1 and CXCR5). Germinal centres are the sites of intense proliferation and somatic mutation to result in differentiation of antigen-activated B cells into high affinity memory B cells or plasma cells. Accordingly, this finding (following antigen injection in the CIA model) is consistent with an impact of clozapine on distal B cell lineage maturation/function and concordant with observations set out in the examples of reduced class switched memory B cells, reduced plasmablast and long-lived plasma cell formation. Together these actions will tend to reduce pathogenic immunoglobulin production in the setting of autoimmune disease.

11. Based on an in vitro differentiation assay, the observed effects of clozapine appear unlikely to reflect a direct effect on antibody secreting cells.

Thus, clozapine appears to have profound influence in vivo on the pathways involved in B cell maturation and pathogenic antibody (particularly pathogenic IgG and IgA antibody) production and thus is useful in treating pathogenic immunoglobulin driven B cell mediated diseases.

These findings are of substantial relevance to the process of IgE production by differentiated B cells based on the ontogeny of IgE B cells and the production of IgE given that: IgE memory B cells and IgE plasma cells also develop via a germinal centre pathway (Talay et al., 2012); IgE switched memory B cells are the main source of cellular IgE memory (Talay et al., 2012); the ontogeny of IgE⁺ B cells and plasma cells follows similar phenotypic stages to that for IgG(1), including IgE⁺ germinal centre-like B cells, IgE plasmablasts and IgE⁺ plasma cells occurring via a sequential switching process from IgG (Ramadani et al., 2017); the intrinsic maturation state of B cells determines their capacity to undergo class switching to IgE with the highest proportion of IgE⁺ cells derive from germinal centre B cells (Ramadani et al., 2017); isotype switching depends on the number of cell divisions and is greater for IgE than IgG (Tangye et al., 2002), consistent with the fact that IgE responses generally require more prolonged antigenic stimulation (Hasbold et al., 1998). Accordingly, clozapine is expected to be useful in treating pathogenic immunoglobulin E (IgE) driven B cell mediated diseases.

### Example 6

### Healthy Human Volunteer Study

This study is a randomized unblinded controlled trial investigating the effects of low-dose clozapine on B cell number and function in healthy volunteers following vaccination (i.e. antigenic challenge). The study employs a parallel arm design (see FIG. 39) with a delayed start for the higher dose tested. In this study a total of up to 48 healthy volunteers will be recruited in to up to 4 cohorts. All participants will be administered Typhi immunization to stimulate the production of specific immunoglobulin (specifically IgG) at day 1 (immunization day) and followed for a period of approximately 56 days. Cohort 1 (n=12 participants) will be administered 25 mg of clozapine for 28 days and followed up for a further 28 days, whilst cohort 2 (n=12 participants, which will be recruited in parallel with Cohort1) will not receive any clozapine but will undergo vaccination. Cohort 2 will be followed in the same manner as cohort 1. Cohort 3 (100 mg clozapine) will only be initiated after the data from the active clozapine treatment period in cohort 1 (day 28 of active treatment) is reviewed by a Safety Committee. There is the potential for an optional cohort of another 12 healthy volunteers to be started if the data warrants further evaluation of doses between 25 and 100 mg clozapine.

Participants in Cohorts 1 and 2 will remain in the trial for a total of 60 days excluding their initial screening visit. Participants in Cohort 3 will take part for a total of 70 days excluding their initial screening visit.

The duration of participation for participants in the optional cohort 4 will vary depending on the dose chosen, due to the titration period being altered accordingly, but excluding their initial screening visit participants will participate for a maximum of 63 days (if a 100 mg dose is selected).

### Objectives and Outcome Measures

| | | Time point(s) of |
|---|---|---|
| | | evaluation of this |
| | | outcome measure |
| Objectives | Outcome Measures | (if applicable) |
| Primary Objective | Difference in specific anti-Typhim Vi | 28 days after |
| To understand the effect of | IgG 28 days after vaccination | vaccination |
| clozapine on primary vaccination | | |
| response | | |
| Secondary Objectives | Change from baseline in total | 28 days after |
| To determine the effect of | immunoglobulin levels (IgG, IgM and | vaccination |
| clozapine on circulating | IgA subclasses) | |
| immunoglobulin levels | | |
| To determine the effect of | Plasmablast response at seven days | 7 days after |
| clozapine on circulating | post-vaccination | vaccination |
| plasmablast levels | | |

| Exploratory Objectives | | |
|---|---|---|
| To understand the exposure- | Concentration response | All available |
| response relationship of | analysis to each primary | timepoints |
| clozapine on B cell subsets | and secondary end point | |
| and immunoglobulins | | |
| Effect of clozapine on | The difference in changes of | 28 days after |
| transcription profiles of | specific RNA expression pre- | vaccination |
| sorted immune cells pre- | clozapine dosing and 28 days | |
| and post-therapy | after vaccination between | |
| | clozapine and control cohorts | |

Similar Immune Biomarkers will be collected in the Healthy Volunteer study to those in the observational study (Example 2).

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents and patent applications referred to herein are incorporated by reference in their entirety.

### REFERENCES

Aalberse, R. C., Platts-Mills, T. A., and Rispens, T. (2016). The Developmental History of IgE and IgG4 Antibodies in Relation to Atopy, Eosinophilic Esophagitis, and the Modified TH2 Response. Current allergy and asthma reports 16, 45.
Alsaleh, G., Francois, A., Knapp, A. M., Schickel, J. N., Sibilia, J., Pasquali, J. L., Gottenberg, J. E., Wachsmann, D., and Soulas-Sprauel, P. (2011). Synovial fibroblasts promote immunoglobulin class switching by a mechanism involving BAFF. European journal of immunology 41, 2113-2122.
Bello, S., Menendez, R., Torres, A., Reyes, S., Zalacain, R., Capelastegui, A., Aspa, J., Borderias, L., Martin-Villasclaras, J. J., Alfageme, I., et al. (2014). Tobacco smoking increases the risk for death from pneumococcal pneumonia. Chest 146, 1029-1037.
Belnoue, E., Pihlgren, M., McGaha, T. L., Tougne, C., Rochat, A. F., Bossen, C., Schneider, P., Huard, B., Lambert, P. H., and Siegrist, C. A. (2008). APRIL is critical for plasmablast survival in the bone marrow and poorly expressed by early-life bone marrow stromal cells. Blood 111, 2755-2764.
Benner, R., Hijmans, W., and Haaijman, J. J. (1981). The bone marrow: the major source of serum immunoglobulins, but still a neglected site of antibody formation. Clinical and experimental immunology 46, 1-8.
Bermejo, D. A., Jackson, S. W., Gorosito-Serran, M., Acosta-Rodriguez, E. V., Amezcua-Vesely, M. C., Sather, B. D., Singh, A. K., Khim, S., Mucci, J., Liggitt, D., et al. (2013). Trypanosoma cruzi trans-sialidase initiates a program independent of the transcription factors RORgammat and Ahr that leads to IL-17 production by activated B cells. Nature immunology 14, 514-522.
Bessis, N., Decker, P., Assier, E., Semerano, L., and Boissier, M. C. (2017). Arthritis models: usefulness and interpretation. Seminars in immunopathology 39, 469-486.
Brand, D. D., Kang, A. H., and Rosloniec, E. F. (2004). The mouse model of collagen-induced arthritis. Methods in molecular medicine 102, 295-312.
Brand, D. D., Latham, K. A., and Rosloniec, E. F. (2007). Collagen-induced arthritis. Nature protocols 2, 1269-1275.
Budeus, B., Schweigle de Reynoso, S., Przekopowitz, M., Hoffmann, D., Seifert, M., and Kuppers, R. (2015). Complexity of the human memory B-cell compartment is determined by the versatility of clonal diversification in germinal centers. Proceedings of the National Academy of Sciences of the United States of America 112, E5281-5289.
Busse, W. W., Morgan, W. J., Gergen, P. J., Mitchell, H. E., Gern, J. E., Liu, A. H., Gruchalla, R. S., Kattan, M., Teach, S. J., Pongracic, J. A., et al. (2011). Randomized trial of omalizumab (anti-IgE) for asthma in inner-city children. The New England journal of medicine 364, 1005-1015.
Campbell, A. M., Vachier, I., Chanez, P., Vignola, A. M., Lebel, B., Kochan, J., Godard, P., and Bousquet, J. (1998). Expression of the high-affinity receptor for IgE on bronchial epithelial cells of asthmatics. American journal of respiratory cell and molecular biology 19, 92-97.
Care, M. A., Stephenson, S. J., Barnes, N. A., Fan, I., Zougman, A., E I-Sherbiny, Y. M., Vital, E. M., Westhead, D. R., Tooze, R. M., and Doody, G. M. (2016). Network Analysis Identifies Proinflammatory Plasma Cell Polarization for Secretion of ISG15 in Human Autoimmunity. Journal of immunology (Baltimore, Md.: 1950) 197, 1447-1459.
Cervenak, L., Magyar, A., Boja, R., and Laszlo, G. (2001). Differential expression of GL7 activation antigen on bone marrow B cell subpopulations and peripheral B cells. Immunology letters 78, 89-96. Chaudhuri, J., and Alt, F. W. (2004). Class-switch recombination: interplay of transcription, DNA deamination and DNA repair. Nature reviews Immunology 4, 541-552.
Chavele, K. M., Merry, E., and Ehrenstein, M. R. (2015). Cutting edge: circulating plasmablasts induce the differentiation of human T follicular helper cells via IL-6 production. Journal of immunology (Baltimore, Md.: 1950) 194, 2482-2485.
Chen, J. W., and Kao, J. Y. (2017). Eosinophilic esophagitis: update on management and controversies. BMJ (Clinical research ed) 359, j4482.
Chen, X., Ma, W., Zhang, T., Wu, L., and Qi, H. (2015). Phenotypic Tfh development promoted by CXCR5-controlled re-localization and IL-6 from radiation-resistant cells. Protein & cell 6, 825-832.
Chu, V. T., and Berek, C. (2013). The establishment of the plasma cell survival niche in the bone marrow. Immunological reviews 251, 177-188.
Chu, V. T., Frohlich, A., Steinhauser, G., Scheel, T., Roch, T., Fillatreau, S., Lee, J. J., Lohning, M., and Berek, C. (2011). Eosinophils are required for the maintenance of plasma cells in the bone marrow. Nature immunology 12, 151-159.
Chua, I., Lagos, M., Charalambous, B. M., Workman, S., Chee, R., and Grimbacher, B. (2011). Pathogen-specific IgG antibody levels in immunodeficient patients receiving immunoglobulin replacement do not provide additional benefit to therapeutic management over total serum IgG. The Journal of allergy and clinical immunology 127, 1410-1411.
Clayton, F., Fang, J. C., Gleich, G. J., Lucendo, A. J., Olalla, J. M., Vinson, L. A., Lowichik, A., Chen, X., Emerson, L., Cox, K., et al. (2014). Eosinophilic esophagitis in adults is associated with IgG4 and not mediated by IgE. Gastroenterology 147, 602-609.
Cocco, M., Stephenson, S., Care, M. A., Newton, D., Barnes, N. A., Davison, A., Rawstron, A., Westhead, D. R., Doody, G. M., and Tooze, R. M. (2012). In vitro generation of long-lived human plasma cells. Journal of immunology (Baltimore, Md.: 1950) 189, 5773-5785.
Dandah, A., Habir, K., Nandakumar, K. S., Saxena, A., Xu, B., Holmdahl, R., and Malin, S. (2018). Germinal Center B Cells Are Essential for Collagen-Induced Arthritis. Arthritis & rheumatology (Hoboken, N.J.) 70, 193-203.
Dellon, E. S., Lin, L., Beitia, R., Moran, T. P., and Qian, Y. (2018). Serum autoantibodies against epithelial cell adhesion molecules as disease biomarkers of eosinophilic esophagitis. Clinical and experimental allergy: journal of the British Society for Allergy and Clinical Immunology 48, 343-346.
Endo, T., Ito, K., Morimoto, J., Kanayama, M., Ota, D., Ikesue, M., Kon, S., Takahashi, D., Onodera, T., Iwasaki, N., et al. (2015). Syndecan 4 Regulation of the Development of Autoimmune Arthritis in Mice by Modulating B Cell Migration and Germinal Center Formation. Arthritis & rheumatology (Hoboken, N.J.) 67, 2512-2522.
Erwin, E. A., Rhoda, D. A., Redmond, M., Ly, J. B., Russo, J. M., Hill, I. D., and Platts-Mills, T. A. E. (2017). Using Serum IgE Antibodies to Predict Esophageal Eosinophilia in Children. Journal of pediatric gastroenterology and nutrition 65, 520-525.
Fahy, J. V., Fleming, H. E., Wong, H. H., Liu, J. T., Su, J. Q., Reimann, J., Fick, R. B., Jr., and Boushey, H. A. (1997). The effect of an anti-IgE monoclonal antibody on the early- and late-phase responses to allergen inhalation in asthmatic subjects. American journal of respiratory and critical care medicine 155, 1828-1834.
Fritz, J. H., Rojas, O. L., Simard, N., McCarthy, D. D., Hapfelmeier, S., Rubino, S., Robertson, S. J., Larijani, M., Gosselin, J., Ivanov, I I, et al. (2011). Acquisition of a multifunctional IgA+ plasma cell phenotype in the gut. Nature 481, 199-203.
Galli, S. J., and Tsai, M. (2012). IgE and mast cells in allergic disease. Nature medicine 18, 693-704. Gray, L., van den Buuse, M., Scarr, E., Dean, B., and Hannan, A. J. (2009). Clozapine reverses schizophrenia-related behaviours in the metabotropic glutamate receptor 5 knockout mouse: association with N-methyl-D-aspartic acid receptor up-regulation. The international journal of neuropsychopharmacology 12, 45-60.
Guitton, C., Kinowski, J. M., Abbar, M., Chabrand, P., and Bressolle, F. (1999). Clozapine and metabolite concentrations during treatment of patients with chronic schizophrenia. Journal of clinical pharmacology 39, 721-728.
Hammarlund, E., Thomas, A., Amanna, I. J., Holden, L. A., Slayden, O. D., Park, B., Gao, L., and Slifka, M. K. (2017). Plasma cell survival in the absence of B cell memory. Nature communications 8, 1781.
Hardtke, S., Ohl, L., and Forster, R. (2005). Balanced expression of CXCR5 and CCR7 on follicular T helper cells determines their transient positioning to lymph node follicles and is essential for efficient B-cell help. Blood 106, 1924-1931.
Haynes, N. M., Allen, C. D., Lesley, R., Ansel, K. M., Killeen, N., and Cyster, J. G. (2007). Role of CXCR5 and CCR7 in follicular Th cell positioning and appearance of a programmed cell death gene-1high germinal center-associated subpopulation. Journal of immunology (Baltimore, Md.: 1950) 179, 5099-5108.
Heeringa, J. J., Rijvers, L., Arends, N. J., Driessen, G. J., Pasmans, S. G., van Dongen, J. J. M., de Jongste, J. C., and van Zelm, M. C. (2018). IgE-expressing memory B cells and plasmablasts are increased in blood of children with asthma, food allergy, and atopic dermatitis. Allergy 73, 1331-1336.
Hiepe, F., Dorner, T., Hauser, A. E., Hoyer, B. F., Mei, H., and Radbruch, A. (2011). Long-lived autoreactive plasma cells drive persistent autoimmune inflammation. Nature reviews Rheumatology 7, 170-178.
Hinze-Selch, D., Becker, E. W., Stein, G. M., Berg, P. A., Mullington, J., Holsboer, F., and Pollmacher, T. (1998). Effects of clozapine on in vitro immune parameters: a longitudinal study in clozapine-treated schizophrenic patients. Neuropsychopharmacology: official publication of the American College of Neuropsychopharmacology 19, 114-122.
Hoyer, B. F., Moser, K., Hauser, A. E., Peddinghaus, A., Voigt, C., Eilat, D., Radbruch, A., Hiepe, F., and Manz, R. A. (2004). Short-lived plasmablasts and long-lived plasma cells contribute to chronic humoral autoimmunity in NZB/W mice. The Journal of experimental medicine 199, 1577-1584.
Humby, F., Bombardieri, M., Manzo, A., Kelly, S., Blades, M. C., Kirkham, B., Spencer, J., and Pitzalis, C. (2009). Ectopic lymphoid structures support ongoing production of class-switched autoantibodies in rheumatoid synovium. PLoS medicine 6, el.
Hung, G. C., Liu, H. C., Yang, S. Y., Pan, C. H., Liao, Y. T., Chen, C. C., and Kuo, C. J. (2016). Antipsychotic reexposure and recurrent pneumonia in schizophrenia: a nested case-control study. The Journal of clinical psychiatry 77, 60-66.
Ishisaka, M., Tsujii, S., Mizoguchi, T., Tsuruma, K., Shimazawa, M., and Hara, H. (2015). The effects of valproate and olanzapine on the abnormal behavior of diacylglycerol kinase beta knockout mice. Pharmacological reports: PR 67, 275-280.
Jolles, S., Borrell, R., Zouwail, S., Heaps, A., Sharp, H., Moody, M., Selwood, C., Williams, P., Phillips, C., Hood, K., et al. (2014). Calculated globulin (C G) as a screening test for antibody deficiency. Clinical and experimental immunology 177, 671-678.
Joshi, R. S., Quadros, R., Drumm, M., Ain, R., and Panicker, M. M. (2017). Sedative effect of Clozapine is a function of 5-HT2A and environmental novelty. European neuropsychopharmacology: the journal of the European College of Neuropsychopharmacology 27, 70-81.
Kappos, L., Hartung, H. P., Freedman, M. S., Boyko, A., Radu, E. W., Mikol, D. D., Lamarine, M., Hyvert, Y., Freudensprung, U., Plitz, T., et al. (2014). Atacicept in multiple sclerosis (ATAMS): a randomised, placebo-controlled, double-blind, phase 2 trial. The Lancet Neurology 13, 353-363.
Kleinau, S., Martinsson, P., and Heyman, B. (2000). Induction and suppression of collagen-induced arthritis is dependent on distinct fcgamma receptors. The Journal of experimental medicine 191, 1611-1616.
Kracker, S., and Durandy, A. (2011). Insights into the B cell specific process of immunoglobulin class switch recombination. Immunology letters 138, 97-103.
Kuo, C. J., Yang, S. Y., Liao, Y. T., Chen, W. J., Lee, W. C., Shau, W. Y., Chang, Y. T., Tsai, S. Y., and Chen, C. C. (2013). Second-generation antipsychotic medications and risk of pneumonia in schizophrenia. Schizophrenia bulletin 39, 648-657.
Leung, J. G., Hasassri, M. E., Barreto, J. N., Nelson, S., and Morgan, R. J., 3rd (2017). Characterization of Admission Types in Medically Hospitalized Patients Prescribed Clozapine. Psychosomatics 58, 164-172.
Li, H., Fang, M., Xu, M., Li, S., Du, J., Li, W., and Chen, H. (2016). Chronic Olanzapine Treatment Induces Disorders of Plasma Fatty Acid Profile in Balb/c Mice: A Potential Mechanism for Olanzapine-Induced Insulin Resistance. PloS one 11, e0167930.
Loizou, D., Enav, B., Komlodi-Pasztor, E., Hider, P., Kim-Chang, J., Noonan, L., Taber, T., Kaushal, S., Limgala, R., Brown, M., et al. (2015). A pilot study of omalizumab in eosinophilic esophagitis. PloS one 10, e0113483.
Lozano, R., Marin, R., Santacruz, M. J., and Pascual, A. (2016). Effect of clozapine on immunoglobulin M plasma levels. Therapeutic advances in psychopharmacology 6, 58-60.
Lupinek, C., Derfler, K., Lee, S., Prikoszovich, T., Movadat, O., Wollmann, E., Cornelius, C., Weber, M., Froschl, R., Selb, R., et al. (2017). Extracorporeal IgE Immunoadsorption in Allergic Asthma: Safety and Efficacy. EBioMedicine 17, 119-133.
Marinkovic, D., Timotijevic, I., Babinski, T., Totic, S., and Paunovic, V. R. (1994). The side-effects of clozapine: a four year follow-up study. Progress in neuro-psychopharmacology & biological psychiatry 18, 537-544.
Matsumoto, M., Baba, A., Yokota, T., Nishikawa, H., Ohkawa, Y., Kayama, H., Kallies, A., Nutt, S. L., Sakaguchi, S., Takeda, K., et al. (2014). Interleukin-10-producing plasmablasts exert regulatory function in autoimmune inflammation. Immunity 41, 1040-1051.
McOmish, C. E., Lira, A., Hanks, J. B., and Gingrich, J. A. (2012). Clozapine-induced locomotor suppression is mediated by 5-HT2A receptors in the forebrain. Neuropsychopharmacology: official publication of the American College of Neuropsychopharmacology 37, 2747-2755.
Melchers, F. (2015). Checkpoints that control B cell development. The Journal of clinical investigation 125, 2203-2210.
Meyer-Hermann, M., Mohr, E., Pelletier, N., Zhang, Y., Victora, G. D., and Toellner, K. M. (2012). A theory of germinal center B cell selection, division, and exit. Cell reports 2, 162-174.
Michel, L., Touil, H., Pikor, N. B., Gommerman, J. L., Prat, A., and Bar-Or, A. (2015). B Cells in the Multiple Sclerosis Central Nervous System: Trafficking and Contribution to CNS-Compartmentalized Inflammation. Frontiers in immunology 6, 636.
Millan, M. J., Audinot, V., Melon, C., and Newman-Tancredi, A. (1995). Evidence that dopamine D3 receptors participate in clozapine-induced hypothermia. European journal of pharmacology 280, 225-229.
Mohammad, N., Avinashi, V., Chan, E., Valiance, B. A., Portales-Casamar, E., and Bush, J. W. (2018). Pediatric Eosinophilic Esophagitis Is Associated With Increased Lamina Propria Immunoglobulin G4-Positive Plasma Cells. Journal of pediatric gastroenterology and nutrition 67, 204-209.
Moreno, J. L., Holloway, T., Umali, A., Rayannavar, V., Sealfon, S. C., and Gonzalez-Maeso, J. (2013). Persistent effects of chronic clozapine on the cellular and behavioral responses to LSD in mice. Psychopharmacology 225, 217-226.
Moschovakis, G. L., Bubke, A., Friedrichsen, M., Falk, C. S., Feederle, R., and Forster, R. (2017). T cell specific Cxcr5 deficiency prevents rheumatoid arthritis. Scientific reports 7, 8933.
Mumtaz, I. M., Hoyer, B. F., Panne, D., Moser, K., Winter, O., Cheng, Q. Y., Yoshida, T., Burmester, G. R., Radbruch, A., Manz, R. A., et al. (2012). Bone marrow of NZB/W mice is the major site for plasma cells resistant to dexamethasone and cyclophosphamide: implications for the treatment of autoimmunity. Journal of autoimmunity 39, 180-188.
Mutlu, O., Gumuslu, E., Ulak, G., Celikyurt, I. K., Kokturk, S., Kir, H. M., Akar, F., and Erden, F. (2012). Effects of fluoxetine, tianeptine and olanzapine on unpredictable chronic mild stress-induced depression-like behavior in mice. Life sciences 91, 1252-1262.
Naito, Y., Takematsu, H., Koyama, S., Miyake, S., Yamamoto, H., Fujinawa, R., Sugai, M., Okuno, Y., Tsujimoto, G., Yamaji, T., et al. (2007). Germinal center marker GL7 probes activation-dependent repression of N-glycolylneuraminic acid, a sialic acid species involved in the negative modulation of B-cell activation. Molecular and cellular biology 27, 3008-3022.
Nandakumar, K. S., Backlund, J., Vestberg, M., and Holmdahl, R. (2004). Collagen type II (CII)-specific antibodies induce arthritis in the absence of T or B cells but the arthritis progression is enhanced by CII-reactive T cells. Arthritis research & therapy 6, R544-550.
Nguyen, D. C., Garimalla, S., Xiao, H., Kyu, S., Albizua, I., Galipeau, J., Chiang, K. Y., Waller, E. K., Wu, R., Gibson, G., et al. (2018). Factors of the bone marrow microniche that support human plasma cell survival and immunoglobulin secretion. Nature communications 9, 3698.
Nutt, S. L., Hodgkin, P. D., Tarlinton, D. M., and Corcoran, L. M. (2015). The generation of antibody-secreting plasma cells. Nature reviews Immunology 15, 160-171.
Oliveria, J. P., Salter, B. M., Phan, S., Obminski, C. D., Munoz, C. E., Smith, S. G., Scime, T., Watson, R. M., Sehmi, R., and Gauvreau, G. M. (2017). Asthmatic subjects with allergy have elevated levels of IgE+ B cells in the airways. The Journal of allergy and clinical immunology 140, 590-593.e599.
Orange, J. S., Ballow, M., Stiehm, E. R., Ballas, Z. K., Chinen, J., De La Morena, M., Kumararatne, D., Harville, T. O., Hesterberg, P., Koleilat, M., et al. (2012). Use and interpretation of diagnostic vaccination in primary immunodeficiency: a working group report of the Basic and Clinical Immunology Interest Section of the American Academy of Allergy, Asthma & Immunology. The Journal of allergy and clinical immunology 130, S1-24.
Palanichamy, A., Apeltsin, L., Kuo, T. C., Sirota, M., Wang, S., Pitts, S. J., Sundar, P. D., Telman, D., Zhao, L. Z., Derstine, M., et al. (2014). Immunoglobulin class-switched B cells form an active immune axis between CNS and periphery in multiple sclerosis. Science translational medicine 6, 248ra106.
Palomares, O., Akdis, M., Martin-Fontecha, M., and Akdis, C. A. (2017). Mechanisms of immune regulation in allergic diseases: the role of regulatory T and B cells. Immunological reviews 278, 219-236.
Petro, J. B., Gerstein, R. M., Lowe, J., Carter, R. S., Shinners, N., and Khan, W. N. (2002). Transitional type 1 and 2 B lymphocyte subsets are differentially responsive to antigen receptor signaling. The Journal of biological chemistry 277, 48009-48019.
Ponsford, M., Castle, D., Tahir, T., Robinson, R., Wade, W., Steven, R., Bramhall, K., Moody, M., Carne, E., Ford, C., et al. (2018). Clozapine is associated with secondary antibody deficiency. The British journal of psychiatry: the journal of mental science, 1-7.
Richez, C., Truchetet, M. E., Schaeverbeke, T., and Bannwarth, B. (2014). Atacicept as an investigated therapy for rheumatoid arthritis. Expert opinion on investigational drugs 23, 1285-1294.
Rojas, O. L., Probstel, A. K., Porfilio, E. A., Wang, A. A., Charabati, M., Sun, T., Lee, D. S. W., Galicia, G., Ramaglia, V., Ward, L. A., et al. (2019). Recirculating Intestinal IgA-Producing Cells Regulate Neuroinflammation via IL-10. Cell 176, 610-624.e618.
Rosenberg, C. E., Mingler, M. K., Caldwell, J. M., Collins, M. H., Fulkerson, P. C., Morris, D. W., Mukkada, V. A., Putnam, P. E., Shoda, T., Wen, T., et al. (2018). Esophageal IgG4 levels correlate with histopathologic and transcriptomic features in eosinophilic esophagitis. Allergy 73, 1892-1901.
Sacchi, S., Novellis, V., Paolone, G., Nuzzo, T., lannotta, M., Belardo, C., Squillace, M., Bolognesi, P., Rosini, E., Motta, Z., et al. (2017). Olanzapine, but not clozapine, increases glutamate release in the prefrontal cortex of freely moving mice by inhibiting D-aspartate oxidase activity. Scientific reports 7, 46288.
Samuels, J., Ng, Y. S., Coupillaud, C., Paget, D., and Meffre, E. (2005). Human B cell tolerance and its failure in rheumatoid arthritis. Annals of the New York Academy of Sciences 1062, 116-126.
Shen, P., and Fillatreau, S. (2015). Antibody-independent functions of B cells: a focus on cytokines. Nature reviews Immunology 15, 441-451.
Shen, P., Roch, T., Lampropoulou, V., O'Connor, R. A., Stervbo, U., Hilgenberg, E., Ries, S., Dang, V. D., Jaimes, Y., Daridon, C., et al. (2014). IL-35-producing B cells are critical regulators of immunity during autoimmune and infectious diseases. Nature 507, 366-370.
Shi, J., Hou, S., Fang, Q., Liu, X., Liu, X., and Qi, H. (2018). PD-1 Controls Follicular T Helper Cell Positioning and Function. Immunity 49, 264-274.e264.
Simon, V. M., Parra, A., Minarro, J., Arenas, M. C., Vinader-Caerols, C., and Aguilar, M. A. (2000). Predicting how equipotent doses of chlorpromazine, haloperidol, sulpiride, raclopride and clozapine reduce locomotor activity in mice. European neuropsychopharmacology: the journal of the European College of Neuropsychopharmacology 10, 159-164.
Stoecker, Z. R., George, W. T., O'Brien, J. B., Jancik, J., Colon, E., and Rasimas, J. J. (2017). Clozapine usage increases the incidence of pneumonia compared with risperidone and the general population: a retrospective comparison of clozapine, risperidone, and the general population in a single hospital over 25 months. International clinical psychopharmacology 32, 155-160.
Stohl, W., Hiepe, F., Latinis, K. M., Thomas, M., Scheinberg, M. A., Clarke, A., Aranow, C., Wellborne, F. R., Abud-Mendoza, C., Hough, D. R., et al. (2012). Belimumab reduces autoantibodies, normalizes low complement levels, and reduces select B cell populations in patients with systemic lupus erythematosus. Arthritis and rheumatism 64, 2328-2337.
Straumann, A., Bauer, M., Fischer, B., Blaser, K., and Simon, H. U. (2001). Idiopathic eosinophilic esophagitis is associated with a T(H)2-type allergic inflammatory response. The Journal of allergy and clinical immunology 108, 954-961.
Stuart, J. M., and Dixon, F. J. (1983). Serum transfer of collagen-induced arthritis in mice. The Journal of experimental medicine 158, 378-392.
Suan, D., Sundling, C., and Brink, R. (2017). Plasma cell and memory B cell differentiation from the germinal center. Current opinion in immunology 45, 97-102.
Sun, W., Meednu, N., Rosenberg, A., Rangel-Moreno, J., Wang, V., Glanzman, J., Owen, T., Zhou, X., Zhang, H., Boyce, B. F., et al. (2018). B cells inhibit bone formation in rheumatoid arthritis by suppressing osteoblast differentiation. Nature communications 9, 5127.
Suurmond, J., Atisha-Fregoso, Y., Marasco, E., Barley, A. N., Ahmed, N., Calderon, S. A., Wong, M. Y., Mackay, M. C., Aranow, C., and Diamond, B. (2018). Loss of an IgG plasma cell checkpoint in patients with lupus. The Journal of allergy and clinical immunology.
Svensson, L., Jirholt, J., Holmdahl, R., and Jansson, L. (1998). B cell-deficient mice do not develop type II collagen-induced arthritis (CIA). Clinical and experimental immunology 111, 521-526.
Takhar, P., Corrigan, C. J., Smurthwaite, L., O'Connor, B. J., Durham, S. R., Lee, T. H., and Gould, H. J. (2007). Class switch recombination to IgE in the bronchial mucosa of atopic and nonatopic patients with asthma. The Journal of allergy and clinical immunology 119, 213-218.
Tanyeri, M. H., Buyukokuroglu, M. E., Tanyeri, P., Mutlu, O., Akar, F. Y., Ulak, G., and Erden, B. F. (2017). Effects of long-term treatment with haloperidol, clozapine and aripiprazole on mice isolated vas deferens. International urology and nephrology 49, 1561-1567.
Tellier, J., and Nutt, S. L. (2018). Plasma cells: the programming of an antibody-secreting machine. European journal of immunology.
Turner, D. L., Verter, J., Turner, R., and Cao, M. (2017). Tissue resident memory B cells established in lungs in allergic asthma. 198, 71.73-71.73.
van Vollenhoven, R. F., Emery, P., Bingham, C. O., 3rd, Keystone, E. C., Fleischmann, R. M., Furst, D. E., Tyson, N., Collinson, N., and Lehane, P. B. (2013). Long-term safety of rituximab in rheumatoid arthritis: 9.5-year follow-up of the global clinical trial programme with a focus on adverse events of interest in R A patients. Annals of the rheumatic diseases 72, 1496-1502.
Vicario, M., Blanchard, C., Stringer, K. F., Collins, M. H., Mingler, M. K., Ahrens, A., Putnam, P. E., Abonia, J. P., Santos, J., and Rothenberg, M. E. (2010). Local B cells and IgE production in the oesophageal mucosa in eosinophilic oesophagitis. Gut 59, 12-20.
Wardemann, H., Yurasov, S., Schaefer, A., Young, J. W., Meffre, E., and Nussenzweig, M. C. (2003). Predominant autoantibody production by early human B cell precursors. Science (New York, N.Y.) 301, 1374-1377.
Wehr, C., Kivioja, T., Schmitt, C., Ferry, B., Witte, T., Eren, E., Vlkova, M., Hernandez, M., Detkova, D., Bos, P. R., et al. (2008). The EUROclass trial: defining subgroups in common variable immunodeficiency. Blood 111, 77-85.
Weinbrand-Goichberg, J., Segal, I., Ovadia, A., Levine, A., and Dalai, I. (2013). Eosinophilic esophagitis: an immune-mediated esophageal disease. Immunologic research 56, 249-260.
Williams, A. A., Ingram, W. M., Levine, S., Resnik, J., Kamel, C. M., Lish, J. R., Elizalde, D. I., Janowski, S. A., Shoker, J., Kozlenkov, A., et al. (2012). Reduced levels of serotonin 2A receptors underlie resistance of Egr3-deficient mice to locomotor suppression by clozapine. Neuropsychopharmacology: official publication of the American College of Neuropsychopharmacology 37, 2285-2298.
Williams, R. O. (2012). What Have We Learned about the Pathogenesis of Rheumatoid Arthritis from TNF-Targeted Therapy? ISRN Immunology 2012, 15.
Wypych, T. P., Marzi, R., Wu, G. F., Lanzavecchia, A., and Sallusto, F. (2018). Role of B cells in T H cell responses in a mouse model of asthma. The Journal of allergy and clinical immunology 141, 1395-1410.
Yanaba, K., Hamaguchi, Y., Venturi, G. M., Steeber, D. A., St Clair, E. W., and Tedder, T. F. (2007). B cell depletion delays collagen-induced arthritis in mice: arthritis induction requires synergy between humoral and cell-mediated immunity. Journal of immunology (Baltimore, Md.: 1950) 179, 1369-1380.
Yen, E. H., Hornick, J. L., Dehlink, E., Dokter, M., Baker, A., Fiebiger, E., and Nurko, S. (2010). Comparative analysis of FcepsilonRl expression patterns in patients with eosinophilic and reflux esophagitis. Journal of pediatric gastroenterology and nutrition 51, 584-592.
Yurasov, S., Wardemann, H., Hammersen, J., Tsuiji, M., Meffre, E., Pascual, V., and Nussenzweig, M. C. (2005). Defective B cell tolerance checkpoints in systemic lupus erythematosus. The Journal of experimental medicine 201, 703-711.
Zehentmeier, S., Roth, K., Cseresnyes, Z., Sercan, O., Horn, K., Niesner, R. A., Chang, H. D., Radbruch, A., and Hauser, A. E. (2014). Static and dynamic components synergize to form a stable survival niche for bone marrow plasma cells. European journal of immunology 44, 2306-2317.
Aalberse, R. C., Platts-Mills, T. A., and Rispens, T. (2016). The Developmental History of IgE and IgG4 Antibodies in Relation to Atopy, Eosinophilic Esophagitis, and the Modified TH2 Response. Current allergy and asthma reports 16, 45.
Abdulahad, W. H., Lepse, N., Stegeman, C. A., Huitema, M. G., Doornbos-van der Meer, B., Tadema, H., Rutgers, A., Limburg, P. C., Kallenberg, C. G., and Heeringa, P. (2013). Increased frequency of circulating IL-21 producing Th-cells in patients with granulomatosis with polyangiitis (GPA). Arthritis research & therapy 15, R70.
Alsaleh, G., Francois, A., Knapp, A. M., Schickel, J. N., Sibilia, J., Pasquali, J. L., Gottenberg, J. E., Wachsmann, D., and Soulas-Sprauel, P. (2011). Synovial fibroblasts promote immunoglobulin class switching by a mechanism involving BAFF. European journal of immunology 41, 2113-2122.
Altrichter, S., Kriehuber, E., Moser, J., Valenta, R., Kopp, T., and Stingl, G. (2008). Serum IgE autoantibodies target keratinocytes in patients with atopic dermatitis. The Journal of investigative dermatology 128, 2232-2239.
Bello, S., Menendez, R., Torres, A., Reyes, S., Zalacain, R., Capelastegui, A., Aspa, J., Borderias, L., Martin-Villasclaras, J. J., Alfageme, I., et al. (2014). Tobacco smoking increases the risk for death from pneumococcal pneumonia. Chest 146, 1029-1037.
Belnoue, E., Pihlgren, M., McGaha, T. L., Tougne, C., Rochat, A. F., Bossen, C., Schneider, P., Huard, B., Lambert, P. H., and Siegrist, C. A. (2008). APRIL is critical for plasmablast survival in the bone marrow and poorly expressed by early-life bone marrow stromal cells. Blood 111, 2755-2764.
Benner, R., Hijmans, W., and Haaijman, J. J. (1981). The bone marrow: the major source of serum immunoglobulins, but still a neglected site of antibody formation. Clinical and experimental immunology 46, 1-8.
Bermejo, D. A., Jackson, S. W., Gorosito-Serran, M., Acosta-Rodriguez, E. V., Amezcua-Vesely, M. C., Sather, B. D., Singh, A. K., Khim, S., Mucci, J., Liggitt, D., et al. (2013). Trypanosoma cruzi trans-sialidase initiates a program independent of the transcription factors RORgammat and Ahr that leads to IL-17 production by activated B cells. Nature immunology 14, 514-522.
Bessis, N., Decker, P., Assier, E., Semerano, L., and Boissier, M. C. (2017). Arthritis models: usefulness and interpretation. Seminars in immunopathology 39, 469-486.
Brand, D. D., Kang, A. H., and Rosloniec, E. F. (2004). The mouse model of collagen-induced arthritis. Methods in molecular medicine 102, 295-312.
Brand, D. D., Latham, K. A., and Rosloniec, E. F. (2007). Collagen-induced arthritis. Nature protocols 2, 1269-1275.
Budeus, B., Schweigle de Reynoso, S., Przekopowitz, M., Hoffmann, D., Seifert, M., and Kuppers, R. (2015). Complexity of the human memory B-cell compartment is determined by the versatility of clonal diversification in germinal centers. Proceedings of the National Academy of Sciences of the United States of America 112, E5281-5289.
Busse, W. W., Morgan, W. J., Gergen, P. J., Mitchell, H. E., Gern, J. E., Liu, A. H., Gruchalla, R. S., Kattan, M., Teach, S. J., Pongracic, J. A., et al. (2011). Randomized trial of omalizumab (anti-IgE) for asthma in inner-city children. The New England journal of medicine 364, 1005-1015.
Cameron, L., Gounni, A. S., Frenkiel, S., Lavigne, F., Vercelli, D., and Hamid, Q. (2003). S epsilon S mu and S epsilon S gamma switch circles in human nasal mucosa following ex vivo allergen challenge: evidence for direct as well as sequential class switch recombination. Journal of immunology (Baltimore, Md.: 1950) 171, 3816-3822.
Cameron, L., Hamid, Q., Wright, E., Nakamura, Y., Christodoulopoulos, P., Muro, S., Frenkiel, S., Lavigne, F., Durham, S., and Gould, H. (2000). Local synthesis of epsilon germline gene transcripts, IL-4, and IL-13 in allergic nasal mucosa after ex vivo allergen exposure. The Journal of allergy and clinical immunology 106, 46-52.
Campbell, A. M., Vachier, I., Chanez, P., Vignola, A. M., Lebel, B., Kochan, J., Godard, P., and Bousquet, J. (1998). Expression of the high-affinity receptor for IgE on bronchial epithelial cells of asthmatics. American journal of respiratory cell and molecular biology 19, 92-97.
Care, M. A., Stephenson, S. J., Barnes, N. A., Fan, I., Zougman, A., E I-Sherbiny, Y. M., Vital, E. M., Westhead, D. R., Tooze, R. M., and Doody, G. M. (2016). Network Analysis Identifies Proinflammatory Plasma Cell Polarization for Secretion of ISG15 in Human Autoimmunity. Journal of immunology (Baltimore, Md.: 1950) 197, 1447-1459.
Cervenak, L., Magyar, A., Boja, R., and Laszlo, G. (2001). Differential expression of GL7 activation antigen on bone marrow B cell subpopulations and peripheral B cells. Immunology letters 78, 89-96.
Chang, T. W., Chen, C., Lin, C. J., Metz, M., Church, M. K., and Maurer, M. (2015). The potential pharmacologic mechanisms of omalizumab in patients with chronic spontaneous urticaria. The Journal of allergy and clinical immunology 135, 337-342.
Chaudhuri, J., and Alt, F. W. (2004). Class-switch recombination: interplay of transcription, DNA deamination and DNA repair. Nature reviews Immunology 4, 541-552.
Chavele, K. M., Merry, E., and Ehrenstein, M. R. (2015). Cutting edge: circulating plasmablasts induce the differentiation of human T follicular helper cells via IL-6 production. Journal of immunology (Baltimore, Md.: 1950) 194, 2482-2485.
Chen, J. W., and Kao, J. Y. (2017). Eosinophilic esophagitis: update on management and controversies. BMJ (Clinical research ed) 359, j4482.
Chen, X., Ma, W., Zhang, T., Wu, L., and Qi, H. (2015). Phenotypic Tfh development promoted by CXCR5-controlled re-localization and IL-6 from radiation-resistant cells. Protein & cell 6, 825-832.
Chu, V. T., and Berek, C. (2013). The establishment of the plasma cell survival niche in the bone marrow. Immunological reviews 251, 177-188.
Chu, V. T., Frohlich, A., Steinhauser, G., Scheel, T., Roch, T., Fillatreau, S., Lee, J. J., Lohning, M., and Berek, C. (2011). Eosinophils are required for the maintenance of plasma cells in the bone marrow. Nature immunology 12, 151-159.
Chua, I., Lagos, M., Charalambous, B. M., Workman, S., Chee, R., and Grimbacher, B. (2011). Pathogen-specific IgG antibody levels in immunodeficient patients receiving immunoglobulin replacement do not provide additional benefit to therapeutic management over total serum IgG. The Journal of allergy and clinical immunology 127, 1410-1411.
Clayton, F., Fang, J. C., Gleich, G. J., Lucendo, A. J., Olalla, J. M., Vinson, L. A., Lowichik, A., Chen, X., Emerson, L., Cox, K., et al. (2014). Eosinophilic esophagitis in adults is associated with IgG4 and not mediated by IgE. Gastroenterology 147, 602-609.
Cocco, M., Stephenson, S., Care, M. A., Newton, D., Barnes, N. A., Davison, A., Rawstron, A., Westhead, D. R., Doody, G. M., and Tooze, R. M. (2012). In vitro generation of long-lived human plasma cells. Journal of immunology (Baltimore, Md.: 1950) 189, 5773-5785.
Coker, H. A., Durham, S. R., and Gould, H. J. (2003). Local somatic hypermutation and class switch recombination in the nasal mucosa of allergic rhinitis patients. Journal of immunology (Baltimore, Md.: 1950) 171, 5602-5610.
Czarnowicki, T., Esaki, H., Gonzalez, J., Renert-Yuval, Y., Brunner, P., Oliva, M., Estrada, Y., Xu, H., Zheng, X., Talasila, S., et al. (2017). Alterations in B-cell subsets in pediatric patients with early atopic dermatitis. The Journal of allergy and clinical immunology 140, 134-144.e139.
Czarnowicki, T., Gonzalez, J., Bonifacio, K. M., Shemer, A., Xiangyu, P., Kunjravia, N., Malajian, D., Fuentes-Duculan, J., Esaki, H., Noda, S., et al. (2016). Diverse activation and differentiation of multiple B-cell subsets in patients with atopic dermatitis but not in patients with psoriasis. The Journal of allergy and clinical immunology 137, 118-129.e115.
Daeschlein, G., Scholz, S., Lutze, S., Eming, R., Arnold, A., Haase, H., Hertl, M., and Junger, M. (2015). Repetitive immunoadsorption cycles for treatment of severe atopic dermatitis. Therapeutic apheresis and dialysis: official peer-reviewed journal of the International Society for Apheresis, the Japanese Society for Apheresis, the Japanese Society for Dialysis Therapy 19, 279-287.
Dandah, A., Habir, K., Nandakumar, K. S., Saxena, A., Xu, B., Holmdahl, R., and Malin, S. (2018). Germinal Center B Cells Are Essential for Collagen-Induced Arthritis. Arthritis & rheumatology (Hoboken, N.J.) 70, 193-203.
Dellon, E. S., Lin, L., Beitia, R., Moran, T. P., and Qian, Y. (2018). Serum autoantibodies against epithelial cell adhesion molecules as disease biomarkers of eosinophilic esophagitis. Clinical and experimental allergy: journal of the British Society for Allergy and Clinical Immunology 48, 343-346. Endo, T., Ito, K., Morimoto, J., Kanayama, M., Ota, D., Ikesue, M., Kon, S., Takahashi, D., Onodera, T., Iwasaki, N., et al. (2015). Syndecan 4 Regulation of the Development of Autoimmune Arthritis in Mice by Modulating B Cell Migration and Germinal Center Formation. Arthritis & rheumatology (Hoboken, N.J.) 67, 2512-2522.
Erwin, E. A., Rhoda, D. A., Redmond, M., Ly, J. B., Russo, J. M., Hill, I. D., and Platts-Mills, T. A. E. (2017). Using Serum IgE Antibodies to Predict Esophageal Eosinophilia in Children. Journal of pediatric gastroenterology and nutrition 65, 520-525.
Fahy, J. V., Fleming, H. E., Wong, H. H., Liu, J. T., Su, J. Q., Reimann, J., Fick, R. B., Jr., and Boushey, H. A. (1997). The effect of an anti-IgE monoclonal antibody on the early- and late-phase responses to allergen inhalation in asthmatic subjects. American journal of respiratory and critical care medicine 155, 1828-1834.
Fritz, J. H., Rojas, O. L., Simard, N., McCarthy, D. D., Hapfelmeier, S., Rubino, S., Robertson, S. J., Larijani, M., Gosselin, J., Ivanov, I I, et al. (2011). Acquisition of a multifunctional IgA+ plasma cell phenotype in the gut. Nature 481, 199-203.
Furue, M., Chiba, T., Tsuji, G., Ulzii, D., Kido-Nakahara, M., Nakahara, T., and Kadono, T. (2017). Atopic dermatitis: immune deviation, barrier dysfunction, IgE autoreactivity and new therapies. Allergology international: official journal of the Japanese Society of Allergology 66, 398-403.
Galli, S. J., and Tsai, M. (2012). IgE and mast cells in allergic disease. Nature medicine 18, 693-704. Gray, L., van den Buuse, M., Scarr, E., Dean, B., and Hannan, A. J. (2009). Clozapine reverses schizophrenia-related behaviours in the metabotropic glutamate receptor 5 knockout mouse: association with N-methyl-D-aspartic acid receptor up-regulation. The international journal of neuropsychopharmacology 12, 45-60.
Greco, A., Rizzo, M. I., De Virgilio, A., Gallo, A., Fusconi, M., Ruoppolo, G., Altissimi, G., and De Vincentiis, M. (2015). Churg-Strauss syndrome. Autoimmunity reviews 14, 341-348.
Groh, M., Pagnoux, C., Baldini, C., Bel, E., Bottero, P., Cottin, V., Dalhoff, K., Dunogue, B., Gross, W., Holle, J., et al. (2015). Eosinophilic granulomatosis with polyangiitis (Churg-Strauss) (EGPA) Consensus Task Force recommendations for evaluation and management. European journal of internal medicine 26, 545-553.
Guitton, C., Kinowski, J. M., Abbar, M., Chabrand, P., and Bressolle, F. (1999). Clozapine and metabolite concentrations during treatment of patients with chronic schizophrenia. Journal of clinical pharmacology 39, 721-728.
Hammarlund, E., Thomas, A., Amanna, I. J., Holden, L. A., Slayden, O. D., Park, B., Gao, L., and Slifka, M. K. (2017). Plasma cell survival in the absence of B cell memory. Nature communications 8, 1781. Hardtke, S., Ohl, L., and Forster, R. (2005). Balanced expression of CXCR5 and CCR7 on follicular T helper cells determines their transient positioning to lymph node follicles and is essential for efficient B-cell help. Blood 106, 1924-1931.
Hasbold, J., Lyons, A. B., Kehry, M. R., and Hodgkin, P. D. (1998). Cell division number regulates IgG1 and IgE switching of B cells following stimulation by CD40 ligand and IL-4. European journal of immunology 28, 1040-1051.
Haynes, N. M., Allen, C. D., Lesley, R., Ansel, K. M., Killeen, N., and Cyster, J. G. (2007). Role of CXCR5 and CCR7 in follicular Th cell positioning and appearance of a programmed cell death gene-1high germinal center-associated subpopulation. Journal of immunology (Baltimore, Md.: 1950) 179, 5099-5108.
Heeringa, J. J., Rijvers, L., Arends, N. J., Driessen, G. J., Pasmans, S. G., van Dongen, J. J. M., de Jongste, J. C., and van Zelm, M. C. (2018). IgE-expressing memory B cells and plasmablasts are increased in blood of children with asthma, food allergy, and atopic dermatitis. Allergy 73, 1331-1336.
Hiepe, F., Dorner, T., Hauser, A. E., Hoyer, B. F., Mei, H., and Radbruch, A. (2011). Long-lived autoreactive plasma cells drive persistent autoimmune inflammation. Nature reviews Rheumatology 7, 170-178.
Higashi, N., Niimi, Y., Aoki, M., and Kawana, S. (2009). Clinical features of antinuclear antibody-positive patients with atopic dermatitis. Journal of Nippon Medical School=Nippon Ika Daigaku zasshi 76, 300-307.
Hinze-Selch, D., Becker, E. W., Stein, G. M., Berg, P. A., Mullington, J., Holsboer, F., and Pollmacher, T. (1998). Effects of clozapine on in vitro immune parameters: a longitudinal study in clozapine-treated schizophrenic patients. Neuropsychopharmacology: official publication of the American College of Neuropsychopharmacology 19, 114-122.
Holgate, S., Casale, T., Wenzel, S., Bousquet, J., Deniz, Y., and Reisner, C. (2005). The anti-inflammatory effects of omalizumab confirm the central role of IgE in allergic inflammation. The Journal of allergy and clinical immunology 115, 459-465.
Hoyer, B. F., Moser, K., Hauser, A. E., Peddinghaus, A., Voigt, C., Eilat, D., Radbruch, A., Hiepe, F., and Manz, R. A. (2004). Short-lived plasmablasts and long-lived plasma cells contribute to chronic humoral autoimmunity in NZB/W mice. The Journal of experimental medicine 199, 1577-1584.
Humby, F., Bombardieri, M., Manzo, A., Kelly, S., Blades, M. C., Kirkham, B., Spencer, J., and Pitzalis, C. (2009). Ectopic lymphoid structures support ongoing production of class-switched autoantibodies in rheumatoid synovium. PLoS medicine 6, el.
Hung, G. C., Liu, H. C., Yang, S. Y., Pan, C. H., Liao, Y. T., Chen, C. C., and Kuo, C. J. (2016). Antipsychotic reexposure and recurrent pneumonia in schizophrenia: a nested case-control study. The Journal of clinical psychiatry 77, 60-66.
Ishii, T., Fujita, T., Matsushita, T., Yanaba, K., Hasegawa, M., Nakashima, H., Ogawa, F., Shimizu, K., Takehara, K., Tedder, T. F., et al. (2009). Establishment of experimental eosinophilic vasculitis by IgE-mediated cutaneous reverse passive arthus reaction. The American journal of pathology 174, 2225-2233.
Ishisaka, M., Tsujii, S., Mizoguchi, T., Tsuruma, K., Shimazawa, M., and Hara, H. (2015). The effects of valproate and olanzapine on the abnormal behavior of diacylglycerol kinase beta knockout mice. Pharmacological reports: PR 67, 275-280.
Jachiet, M., Samson, M., Cottin, V., Kahn, J. E., Le Guenno, G., Bonniaud, P., Devilliers, H., Bouillet, L., Gondouin, A., Makhlouf, F., et al. (2016). Anti-IgE Monoclonal Antibody (Omalizumab) in Refractory and Relapsing Eosinophilic Granulomatosis With Polyangiitis (Churg-Strauss): Data on Seventeen Patients. Arthritis & rheumatology (Hoboken, N.J.) 68, 2274-2282.
Jolles, S., Borrell, R., Zouwail, S., Heaps, A., Sharp, H., Moody, M., Selwood, C., Williams, P., Phillips, C., Hood, K., et al. (2014). Calculated globulin (C G) as a screening test for antibody deficiency. Clinical and experimental immunology 177, 671-678.
Joshi, R. S., Quadros, R., Drumm, M., Ain, R., and Panicker, M. M. (2017). Sedative effect of Clozapine is a function of 5-HT2A and environmental novelty. European neuropsychopharmacology: the journal of the European College of Neuropsychopharmacology 27, 70-81.
Kappos, L., Hartung, H. P., Freedman, M. S., Boyko, A., Radu, E. W., Mikol, D. D., Lamarine, M., Hyvert, Y., Freudensprung, U., Plitz, T., et al. (2014). Atacicept in multiple sclerosis (ATAMS): a randomised, placebo-controlled, double-blind, phase 2 trial. The Lancet Neurology 13, 353-363.
Kleinau, S., Martinsson, P., and Heyman, B. (2000). Induction and suppression of collagen-induced arthritis is dependent on distinct fcgamma receptors. The Journal of experimental medicine 191, 1611-1616.
KleinJan, A., Vinke, J. G., Severijnen, L. W., and Fokkens, W. J. (2000). Local production and detection of (specific) IgE in nasal B-cells and plasma cells of allergic rhinitis patients. The European respiratory journal 15, 491-497.
Kopp, M. V., Hamelmann, E., Zielen, S., Kamin, W., Bergmann, K. C., Sieder, C., Stenglein, S., Seyfried, S., and Wahn, U. (2009). Combination of omalizumab and specific immunotherapy is superior to immunotherapy in patients with seasonal allergic rhinoconjunctivitis and co-morbid seasonal allergic asthma. Clinical and experimental allergy: journal of the British Society for Allergy and Clinical Immunology 39, 271-279.
Kracker, S., and Durandy, A. (2011). Insights into the B cell specific process of immunoglobulin class switch recombination. Immunology letters 138, 97-103.
Kuo, C. J., Yang, S. Y., Liao, Y. T., Chen, W. J., Lee, W. C., Shau, W. Y., Chang, Y. T., Tsai, S. Y., and Chen, C. C. (2013). Second-generation antipsychotic medications and risk of pneumonia in schizophrenia. Schizophrenia bulletin 39, 648-657.
Leung, J. G., Hasassri, M. E., Barreto, J. N., Nelson, S., and Morgan, R. J., 3rd (2017). Characterization of Admission Types in Medically Hospitalized Patients Prescribed Clozapine. Psychosomatics 58, 164-172.
Li, H., Fang, M., Xu, M., Li, S., Du, J., Li, W., and Chen, H. (2016). Chronic Olanzapine Treatment Induces Disorders of Plasma Fatty Acid Profile in Balb/c Mice: A Potential Mechanism for Olanzapine-Induced Insulin Resistance. PloS one 11, e0167930.
Loizou, D., Enav, B., Komlodi-Pasztor, E., Hider, P., Kim-Chang, J., Noonan, L., Taber, T., Kaushal, S., Limgala, R., Brown, M., et al. (2015). A pilot study of omalizumab in eosinophilic esophagitis. PloS one 10, e0113483.
Lozano, R., Marin, R., Santacruz, M. J., and Pascual, A. (2016). Effect of clozapine on immunoglobulin M plasma levels. Therapeutic advances in psychopharmacology 6, 58-60.
Lupinek, C., Derfler, K., Lee, S., Prikoszovich, T., Movadat, O., Wollmann, E., Cornelius, C., Weber, M., Froschl, R., Selb, R., et al. (2017). Extracorporeal IgE Immunoadsorption in Allergic Asthma: Safety and Efficacy. EBioMedicine 17, 119-133.
Manger, B. J., Krapf, F. E., Gramatzki, M., Nusslein, H. G., Burmester, G. R., Krauledat, P. B., and Kalden, J. R. (1985). IgE-containing circulating immune complexes in Churg-Strauss vasculitis. Scandinavian journal of immunology 21, 369-373.
Marinkovic, D., Timotijevic, I., Babinski, T., Totic, S., and Paunovic, V. R. (1994). The side-effects of clozapine: a four year follow-up study. Progress in neuro-psychopharmacology & biological psychiatry 18, 537-544.
Matsumoto, M., Baba, A., Yokota, T., Nishikawa, H., Ohkawa, Y., Kayama, H., Kallies, A., Nutt, S. L., Sakaguchi, S., Takeda, K., et al. (2014). Interleukin-10-producing plasmablasts exert regulatory function in autoimmune inflammation. Immunity 41, 1040-1051.
McOmish, C. E., Lira, A., Hanks, J. B., and Gingrich, J. A. (2012). Clozapine-induced locomotor suppression is mediated by 5-HT2A receptors in the forebrain. Neuropsychopharmacology: official publication of the American College of Neuropsychopharmacology 37, 2747-2755.
Melchers, F. (2015). Checkpoints that control B cell development. The Journal of clinical investigation 125, 2203-2210.
Meyer-Hermann, M., Mohr, E., Pelletier, N., Zhang, Y., Victora, G. D., and Toellner, K. M. (2012). A theory of germinal center B cell selection, division, and exit. Cell reports 2, 162-174.
Michel, L., Touil, H., Pikor, N. B., Gommerman, J. L., Prat, A., and Bar-Or, A. (2015). B Cells in the Multiple Sclerosis Central Nervous System: Trafficking and Contribution to CNS-Compartmentalized Inflammation. Frontiers in immunology 6, 636.
Millan, M. J., Audinot, V., Melon, C., and Newman-Tancredi, A. (1995). Evidence that dopamine D3 receptors participate in clozapine-induced hypothermia. European journal of pharmacology 280, 225-229.
Mohammad, A. J., Hot, A., Arndt, F., Moosig, F., Guerry, M. J., Amudala, N., Smith, R., Sivasothy, P., Guillevin, L., Merkel, P. A., et al. (2016). Rituximab for the treatment of eosinophilic granulomatosis with polyangiitis (Churg-Strauss). Annals of the rheumatic diseases 75, 396-401.
Mohammad, N., Avinashi, V., Chan, E., Valiance, B. A., Portales-Casamar, E., and Bush, J. W. (2018). Pediatric Eosinophilic Esophagitis Is Associated With Increased Lamina Propria Immunoglobulin G4-Positive Plasma Cells. Journal of pediatric gastroenterology and nutrition 67, 204-209.
Mohan, G. C., and Silverberg, J. I. (2015). Association of Vitiligo and Alopecia Areata With Atopic Dermatitis: A Systematic Review and Meta-analysis. JAMA dermatology 151, 522-528.
Moreno, J. L., Holloway, T., Umali, A., Rayannavar, V., Sealfon, S. C., and Gonzalez-Maeso, J. (2013). Persistent effects of chronic clozapine on the cellular and behavioral responses to LSD in mice. Psychopharmacology 225, 217-226.
Moschovakis, G. L., Bubke, A., Friedrichsen, M., Falk, C. S., Feederle, R., and Forster, R. (2017). T cell specific Cxcr5 deficiency prevents rheumatoid arthritis. Scientific reports 7, 8933.
Mumtaz, I. M., Hoyer, B. F., Panne, D., Moser, K., Winter, O., Cheng, Q. Y., Yoshida, T., Burmester, G. R., Radbruch, A., Manz, R. A., et al. (2012). Bone marrow of NZB/W mice is the major site for plasma cells resistant to dexamethasone and cyclophosphamide: implications for the treatment of autoimmunity. Journal of autoimmunity 39, 180-188.
Mutlu, O., Gumuslu, E., Ulak, G., Celikyurt, I. K., Kokturk, S., Kir, H. M., Akar, F., and Erden, F. (2012). Effects of fluoxetine, tianeptine and olanzapine on unpredictable chronic mild stress-induced depression-like behavior in mice. Life sciences 91, 1252-1262.
Naito, Y., Takematsu, H., Koyama, S., Miyake, S., Yamamoto, H., Fujinawa, R., Sugai, M., Okuno, Y., Tsujimoto, G., Yamaji, T., et al. (2007). Germinal center marker GL7 probes activation-dependent repression of N-glycolylneuraminic acid, a sialic acid species involved in the negative modulation of B-cell activation. Molecular and cellular biology 27, 3008-3022.
Nakazawa, D., Masuda, S., Tomaru, U., and Ishizu, A. (2019). Pathogenesis and therapeutic interventions for ANCA-associated vasculitis. Nature reviews Rheumatology 15, 91-101.
Nandakumar, K. S., Backlund, J., Vestberg, M., and Holmdahl, R. (2004). Collagen type II (CII)-specific antibodies induce arthritis in the absence of T or B cells but the arthritis progression is enhanced by CII-reactive T cells. Arthritis research & therapy 6, R544-550.
Navarrete-Dechent, C., Perez-Mateluna, G., Silva-Valenzuela, S., Vera-Kellet, C., and Borzutzky, A. (2016). Humoral and Cellular Autoreactivity to Epidermal Proteins in Atopic Dermatitis. Archivum immunologiae et therapiae experimentalis 64, 435-442.
Nguyen, D. C., Garimalla, S., Xiao, H., Kyu, S., Albizua, I., Galipeau, J., Chiang, K. Y., Waller, E. K., Wu, R., Gibson, G., et al. (2018). Factors of the bone marrow microniche that support human plasma cell survival and immunoglobulin secretion. Nature communications 9, 3698.
Nutt, S. L., Hodgkin, P. D., Tarlinton, D. M., and Corcoran, L. M. (2015). The generation of antibody-secreting plasma cells. Nature reviews Immunology 15, 160-171.
Oliveria, J. P., Salter, B. M., Phan, S., Obminski, C. D., Munoz, C. E., Smith, S. G., Scime, T., Watson, R. M., Sehmi, R., and Gauvreau, G. M. (2017). Asthmatic subjects with allergy have elevated levels of IgE+ B cells in the airways. The Journal of allergy and clinical immunology 140, 590-593.e599.
Orange, J. S., Ballow, M., Stiehm, E. R., Ballas, Z. K., Chinen, J., De La Morena, M., Kumararatne, D., Harville, T. O., Hesterberg, P., Koleilat, M., et al. (2012). Use and interpretation of diagnostic vaccination in primary immunodeficiency: a working group report of the Basic and Clinical Immunology Interest Section of the American Academy of Allergy, Asthma & Immunology. The Journal of allergy and clinical immunology 130, S1-24.
Palanichamy, A., Apeltsin, L., Kuo, T. C., Sirota, M., Wang, S., Pitts, S. J., Sundar, P. D., Telman, D., Zhao, L. Z., Derstine, M., et al. (2014). Immunoglobulin class-switched B cells form an active immune axis between CNS and periphery in multiple sclerosis. Science translational medicine 6, 248ra106.
Palomares, O., Akdis, M., Martin-Fontecha, M., and Akdis, C. A. (2017). Mechanisms of immune regulation in allergic diseases: the role of regulatory T and B cells. Immunological reviews 278, 219-236.
Pawankar, R., and Ra, C. (1998). IgE-Fc epsilonRI-mast cell axis in the allergic cycle. Clinical and experimental allergy: journal of the British Society for Allergy and Clinical Immunology 28 Suppl 3, 6-14.
Pene, J. (1989). Regulatory role of cytokines and CD23 in the human IgE antibody synthesis. International archives of allergy and applied immunology 90 Suppl 1, 32-40.
Petro, J. B., Gerstein, R. M., Lowe, J., Carter, R. S., Shinners, N., and Khan, W. N. (2002). Transitional type 1 and 2 B lymphocyte subsets are differentially responsive to antigen receptor signaling. The Journal of biological chemistry 277, 48009-48019.
Ponsford, M., Castle, D., Tahir, T., Robinson, R., Wade, W., Steven, R., Bramhall, K., Moody, M., Carne, E., Ford, C., et al. (2018a). Clozapine is associated with secondary antibody deficiency. The British Journal of Psychiatry, 1-7.
Ponsford, M., Castle, D., Tahir, T., Robinson, R., Wade, W., Steven, R., Bramhall, K., Moody, M., Carne, E., Ford, C., et al. (2018b). Clozapine is associated with secondary antibody deficiency. The British journal of psychiatry: the journal of mental science, 1-7.
Radonjic-Hoesli, S., Hofmeier, K. S., Micaletto, S., Schmid-Grendelmeier, P., Bircher, A., and Simon, D. (2018). Urticaria and Angioedema: an Update on Classification and Pathogenesis. Clinical reviews in allergy & immunology 54, 88-101.
Ramadani, F., Bowen, H., Upton, N., Hobson, P. S., Chan, Y. C., Chen, J. B., Chang, T. W., McDonnell, J. M., Sutton, B. J., Fear, D. J., et al. (2017). Ontogeny of human IgE-expressing B cells and plasma cells. Allergy 72, 66-76.
Richez, C., Truchetet, M. E., Schaeverbeke, T., and Bannwarth, B. (2014). Atacicept as an investigated therapy for rheumatoid arthritis. Expert opinion on investigational drugs 23, 1285-1294.
Rojas, O. L., Probstel, A. K., Porfilio, E. A., Wang, A. A., Charabati, M., Sun, T., Lee, D. S. W., Galicia, G., Ramaglia, V., Ward, L. A., et al. (2019). Recirculating Intestinal IgA-Producing Cells Regulate Neuroinflammation via IL-10. Cell 176, 610-624.e618.
Rosenberg, C. E., Mingler, M. K., Caldwell, J. M., Collins, M. H., Fulkerson, P. C., Morris, D. W., Mukkada, V. A., Putnam, P. E., Shoda, T., Wen, T., et al. (2018). Esophageal IgG4 levels correlate with histopathologic and transcriptomic features in eosinophilic esophagitis. Allergy 73, 1892-1901.
Sacchi, S., Novellis, V., Paolone, G., Nuzzo, T., lannotta, M., Belardo, C., Squillace, M., Bolognesi, P., Rosini, E., Motta, Z., et al. (2017). Olanzapine, but not clozapine, increases glutamate release in the prefrontal cortex of freely moving mice by inhibiting D-aspartate oxidase activity. Scientific reports 7, 46288.
Samuels, J., Ng, Y. S., Coupillaud, C., Paget, D., and Meffre, E. (2005). Human B cell tolerance and its failure in rheumatoid arthritis. Annals of the New York Academy of Sciences 1062, 116-126.
Sanchez-Ramon, S., Eguiluz-Gracia, I., Rodriguez-Mazariego, M. E., Paravisini, A., Zubeldia-Ortuno, J. M., Gil-Herrera, J., Fernandez-Cruz, E., and Suarez-Fernandez, R. (2013). Sequential combined therapy with omalizumab and rituximab: a new approach to severe atopic dermatitis. Journal of investigational allergology & clinical immunology 23, 190-196.
Schmid-Grendelmeier, P., Fluckiger, S., Disch, R., Trautmann, A., Wuthrich, B., Blaser, K., Scheynius, A., and Crameri, R. (2005). IgE-mediated and T cell-mediated autoimmunity against manganese superoxide dismutase in atopic dermatitis. The Journal of allergy and clinical immunology 115, 1068-1075.
Shen, P., and Fillatreau, S. (2015). Antibody-independent functions of B cells: a focus on cytokines. Nature reviews Immunology 15, 441-451.
Shen, P., Roch, T., Lampropoulou, V., O'Connor, R. A., Stervbo, U., Hilgenberg, E., Ries, S., Dang, V. D., Jaimes, Y., Daridon, C., et al. (2014). IL-35-producing B cells are critical regulators of immunity during autoimmune and infectious diseases. Nature 507, 366-370.
Shi, J., Hou, S., Fang, Q., Liu, X., Liu, X., and Qi, H. (2018). PD-1 Controls Follicular T Helper Cell Positioning and Function. Immunity 49, 264-274.e264.
Simon, D., Hosli, S., Kostylina, G., Yawalkar, N., and Simon, H. U. (2008). Anti-CD20 (rituximab) treatment improves atopic eczema. The Journal of allergy and clinical immunology 121, 122-128.
Simon, V. M., Parra, A., Minarro, J., Arenas, M. C., Vinader-Caerols, C., and Aguilar, M. A. (2000). Predicting how equipotent doses of chlorpromazine, haloperidol, sulpiride, raclopride and clozapine reduce locomotor activity in mice. European neuropsychopharmacology: the journal of the European College of Neuropsychopharmacology 10, 159-164.
Stoecker, Z. R., George, W. T., O'Brien, J. B., Jancik, J., Colon, E., and Rasimas, J. J. (2017). Clozapine usage increases the incidence of pneumonia compared with risperidone and the general population: a retrospective comparison of clozapine, risperidone, and the general population in a single hospital over 25 months. International clinical psychopharmacology 32, 155-160.
Stohl, W., Hiepe, F., Latinis, K. M., Thomas, M., Scheinberg, M. A., Clarke, A., Aranow, C., Wellborne, F. R., Abud-Mendoza, C., Hough, D. R., et al. (2012). Belimumab reduces autoantibodies, normalizes low complement levels, and reduces select B cell populations in patients with systemic lupus erythematosus. Arthritis and rheumatism 64, 2328-2337.
Straumann, A., Bauer, M., Fischer, B., Blaser, K., and Simon, H. U. (2001). Idiopathic eosinophilic esophagitis is associated with a T(H)2-type allergic inflammatory response. The Journal of allergy and clinical immunology 108, 954-961.
Stuart, J. M., and Dixon, F. J. (1983). Serum transfer of collagen-induced arthritis in mice. The Journal of experimental medicine 158, 378-392.
Suan, D., Sundling, C., and Brink, R. (2017). Plasma cell and memory B cell differentiation from the germinal center. Current opinion in immunology 45, 97-102.
Sun, W., Meednu, N., Rosenberg, A., Rangel-Moreno, J., Wang, V., Glanzman, J., Owen, T., Zhou, X., Zhang, H., Boyce, B. F., et al. (2018). B cells inhibit bone formation in rheumatoid arthritis by suppressing osteoblast differentiation. Nature communications 9, 5127.
Suurmond, J., Atisha-Fregoso, Y., Marasco, E., Barley, A. N., Ahmed, N., Calderon, S. A., Wong, M. Y., Mackay, M. C., Aranow, C., and Diamond, B. (2018). Loss of an IgG plasma cell checkpoint in patients with lupus. The Journal of allergy and clinical immunology.
Svensson, L., Jirholt, J., Holmdahl, R., and Jansson, L. (1998). B cell-deficient mice do not develop type II collagen-induced arthritis (CIA). Clinical and experimental immunology 111, 521-526.
Takhar, P., Corrigan, C. J., Smurthwaite, L., O'Connor, B. J., Durham, S. R., Lee, T. H., and Gould, H. J. (2007). Class switch recombination to IgE in the bronchial mucosa of atopic and nonatopic patients with asthma. The Journal of allergy and clinical immunology 119, 213-218.
Takhar, P., Smurthwaite, L., Coker, H. A., Fear, D. J., Banfield, G. K., Carr, V. A., Durham, S. R., and Gould, H. J. (2005). Allergen drives class switching to IgE in the nasal mucosa in allergic rhinitis. Journal of immunology (Baltimore, Md.: 1950) 174, 5024-5032.
Talay, O., Yan, D., Brightbill, H. D., Straney, E. E., Zhou, M., Ladi, E., Lee, W. P., Egen, J. G., Austin, C. D., Xu, M., et al. (2012). IgE(+) memory B cells and plasma cells generated through a germinal-center pathway. Nature immunology 13, 396-404.
Tangye, S. G., Ferguson, A., Avery, D. T., Ma, C. S., and Hodgkin, P. D. (2002). Isotype switching by human B cells is division-associated and regulated by cytokines. Journal of immunology (Baltimore, Md.: 1950) 169, 4298-4306.
Tanyeri, M. H., Buyukokuroglu, M. E., Tanyeri, P., Mutlu, O., Akar, F. Y., Ulak, G., and Erden, B. F. (2017). Effects of long-term treatment with haloperidol, clozapine and aripiprazole on mice isolated vas deferens. International urology and nephrology 49, 1561-1567.
Tellier, J., and Nutt, S. L. (2018). Plasma cells: the programming of an antibody-secreting machine. European journal of immunology.
Thiel, J., Troilo, A., Salzer, U., Schleyer, T., Halmschlag, K., Rizzi, M., Frede, N., Venhoff, A., Voll, R. E., and Venhoff, N. (2017). Rituximab as Induction Therapy in Eosinophilic Granulomatosis with Polyangiitis Refractory to Conventional Immunosuppressive Treatment: A 36-Month Follow-Up Analysis. The journal of allergy and clinical immunology In practice 5, 1556-1563.
Thomas, P., Pfutzner, W., and Przybilla, B. (1995). Frequency and activity of IgE-secreting peripheral blood B-cells in atopic eczema. Acta dermato-venereologica 75, 422-425.
Tsabouri, S., Tseretopoulou, X., Priftis, K., and Ntzani, E. E. (2014). Omalizumab for the treatment of inadequately controlled allergic rhinitis: a systematic review and meta-analysis of randomized clinical trials. The journal of allergy and clinical immunology in practice 2, 332-340.e331.
Tsurikisawa, N., Saito, H., Oshikata, C., Tsuburai, T., and Akiyama, K. (2013). Decreases in the numbers of peripheral blood regulatory T cells, and increases in the levels of memory and activated B cells, in patients with active eosinophilic granulomatosis and polyangiitis. Journal of clinical immunology 33, 965-976.
Turner, D. L., Verter, J., Turner, R., and Cao, M. (2017). Tissue resident memory B cells established in lungs in allergic asthma. 198, 71.73-71.73.
van Vollenhoven, R. F., Emery, P., Bingham, C. O., 3rd, Keystone, E. C., Fleischmann, R. M., Furst, D. E., Tyson, N., Collinson, N., and Lehane, P. B. (2013). Long-term safety of rituximab in rheumatoid arthritis: 9.5-year follow-up of the global clinical trial programme with a focus on adverse events of interest in R A patients. Annals of the rheumatic diseases 72, 1496-1502.
Vicario, M., Blanchard, C., Stringer, K. F., Collins, M. H., Mingler, M. K., Ahrens, A., Putnam, P. E., Abonia, J. P., Santos, J., and Rothenberg, M. E. (2010). Local B cells and IgE production in the oesophageal mucosa in eosinophilic oesophagitis. Gut 59, 12-20.
Wardemann, H., Yurasov, S., Schaefer, A., Young, J. W., Meffre, E., and Nussenzweig, M. C. (2003). Predominant autoantibody production by early human B cell precursors. Science (New York, N.Y.) 301, 1374-1377.
Wehr, C., Kivioja, T., Schmitt, C., Ferry, B., Witte, T., Eren, E., Vlkova, M., Hernandez, M., Detkova, D., Bos, P. R., et al. (2008). The EUROclass trial: defining subgroups in common variable immunodeficiency. Blood 111, 77-85.
Weinbrand-Goichberg, J., Segal, I., Ovadia, A., Levine, A., and Dalai, I. (2013). Eosinophilic esophagitis: an immune-mediated esophageal disease. Immunologic research 56, 249-260.
Williams, A. A., Ingram, W. M., Levine, S., Resnik, J., Kamel, C. M., Lish, J. R., Elizalde, D. I., Janowski, S. A., Shoker, J., Kozlenkov, A., et al. (2012). Reduced levels of serotonin 2A receptors underlie resistance of Egr3-deficient mice to locomotor suppression by clozapine. Neuropsychopharmacology: official publication of the American College of Neuropsychopharmacology 37, 2285-2298.
Williams, R. O. (2012). What Have We Learned about the Pathogenesis of Rheumatoid Arthritis from TNF-Targeted Therapy? ISRN Immunology 2012, 15.
Wise, S. K., Lin, S. Y., Toskala, E., Orlandi, R. R., Akdis, C. A., Alt, J. A., Azar, A., Baroody, F. M., Bachert, C., Canonica, G. W., et al. (2018). International Consensus Statement on Allergy and Rhinology: Allergic Rhinitis. International forum of allergy & rhinology 8, 108-352.
Wypych, T. P., Marzi, R., Wu, G. F., Lanzavecchia, A., and Sallusto, F. (2018). Role of B cells in T H cell responses in a mouse model of asthma. The Journal of allergy and clinical immunology 141, 1395-1410.
Yanaba, K., Hamaguchi, Y., Venturi, G. M., Steeber, D. A., St Clair, E. W., and Tedder, T. F. (2007). B cell depletion delays collagen-induced arthritis in mice: arthritis induction requires synergy between humoral and cell-mediated immunity. Journal of immunology (Baltimore, Md.: 1950) 179, 1369-1380.
Yen, E. H., Hornick, J. L., Dehlink, E., Dokter, M., Baker, A., Fiebiger, E., and Nurko, S. (2010). Comparative analysis of FcepsilonRl expression patterns in patients with eosinophilic and reflux esophagitis. Journal of pediatric gastroenterology and nutrition 51, 584-592.
Yurasov, S., Wardemann, H., Hammersen, J., Tsuiji, M., Meffre, E., Pascual, V., and Nussenzweig, M. C. (2005). Defective B cell tolerance checkpoints in systemic lupus erythematosus. The Journal of experimental medicine 201, 703-711.
Zehentmeier, S., Roth, K., Cseresnyes, Z., Sercan, O., Horn, K., Niesner, R. A., Chang, H. D., Radbruch, A., and Hauser, A. E. (2014). Static and dynamic components synergize to form a stable survival niche for bone marrow plasma cells. European journal of immunology 44, 2306-2317.
Zheng, T., Yu, J., Oh, M. H., and Zhu, Z. (2011). The atopic march: progression from atopic dermatitis to allergic rhinitis and asthma. Allergy, asthma & immunology research 3, 67-73.

1. (canceled)

2. A method of treatment or prevention of a pathogenic IgE driven B cell disease in a subject by administering to said subject an effective amount of a compound selected from clozapine, norclozapine and prodrugs thereof and pharmaceutically acceptable salts and solvates thereof wherein said compound causes mature B cells to be inhibited in said subject.

3. (canceled)

4. The method according to claim 2 wherein the compound is clozapine or a pharmaceutically acceptable salt or solvate thereof.

5. The method according to claim 2 wherein the mature B cells are class switched memory B-cells.

6. The method according to claim 2 wherein the mature B cells are plasmablasts.

7. The method according to claim 2 wherein the pathogenic IgE driven B cell disease is a disease selected from the group consisting of atopic asthma, atopic dermatitis, chronic non-autoimmune urticaria, Churg-Strauss vasculitis, allergic rhinitis and allergic eye disease preferably atopic dermatitis, atopic asthma, allergic rhinitis and eosinophilic esophagitis.

8. The method according to claim 2 wherein the compound has the effect of decreasing CD19 (+) B cells and/or (−) B plasma cells.

9. A method of treatment or prevention of a pathogenic IgE driven B cell disease in a subject comprising administering to the subject an effective amount of a pharmaceutical composition comprising a compound selected from clozapine, norclozapine and prodrugs thereof and pharmaceutically acceptable salts and solvates thereof; and a pharmaceutically acceptable diluent or carrier wherein said compound causes mature B cells to be inhibited in said subject.

10. The method according to claim 9 wherein the pharmaceutical composition is administered orally.

11. The method according to claim 9 wherein the pharmaceutical composition is formulated as a liquid or solid, such as a syrup, suspension, emulsion, tablets, capsule or lozenge.

12. The method according to claim 9 wherein the mature B-cells are class switched memory B cells.

13. The method according to claim 9 wherein the mature B-cells are plasmablasts.

14. A method according to claim 1, wherein the compound is administered in combination with a second or further therapeutic agent for the treatment or prevention of a pathogenic IgE driven B cell disease.

15. The method according to claim 14 wherein the second or further substance for the treatment or prevention of a pathogenic IgE driven B cell disease is selected from anti-TNFα agents (such as anti-TNFα antibodies e.g. infliximab or adalumumab), calcineurin inhibitors (such as tacrolimus or cyclosporine), antiproliferative agents (such as mycophenolate e.g. as mofetil or sodium, or azathioprine), general anti-inflammatories (such as hydroxychloroquine or NSAIDS such as ketoprofen and colchicine), mTOR inhibitors (such as sirolimus), steroids (such as prednisone), anti-CD80/CD86 agents (such as abatacept), anti-CD-20 agents (such as anti-CD-20 antibodies e.g. rituximab), anti-BAFF agents (such as anti-BAFF antibodies e.g. tabalumab or belimumab, or atacicept), immunosuppressants (such as methotrexate or cyclophosphamide), anti-FcRn agents (e.g. anti-FcRn antibodies) and other antibodies (such as ARGX-113, PRN-1008, SYNT-001, veltuzumab, ocrelizumab, ofatumumab, obinutuzumab, ublituximab, alemtuzumab, milatuzumab, epratuzumab and blinatumomab).
